(19) United States
(12) Patent Application Publication
(10) Pub. No.: US 2013/0310347 A1
(43) Pub. Date: Nov. 21, 2013

(54) MULTI-MODALITY MOLECULAR IMAGING HIGH-THROUGHPUT ASSAY FOR IDENTIFYING HEAT SHOCK PROTEIN 90 (HSP90) INHIBITORS

(71) Applicant: The Board of Trustees of the Leland Stanford Junior University, Palo Alto, CA (US)

(72) Inventors: Carmel Chan, Santa Clara, CA (US); Sanjiv S. Gambhir, Portola Valley, CA (US); David E. Solow-Cordero, San Francisco, CA (US); Aileen Hoehne, Fremont, CA (US); Ramasamy Paulmurugan, Stanford, CA (US)

(73) Assignee: The Board of Trustees of the Leland Stanford Junior University, Palo Alto, CA (US)

(21) Appl. No.: 13/898,637

(22) Filed: May 21, 2013

Related U.S. Application Data
(60) Provisional application No. 61/649,560, filed on May 21, 2012.
(60) Provisional application No. 61/663,763, filed on Jun. 25, 2012.

(51) Int. Cl. C07D 473/08; C07C 311/38; C07D 413/14; C07D 213/75; C07D 213/65; C07D 213/42; C07D 513/04; C07D 213/81

(52) U.S. Cl. CPC C07D 473/08; C07D 513/04; C07C 311/38; C07D 213/81; C07D 213/75; C07D 213/65; C07D 213/42; C07D 413/14
USPC 514/156; 544/273; 514/263.34; 548/154; 514/368; 564/86; 514/155; 546/262; 514/332; 546/309; 514/352; 546/291; 514/346; 544/267; 546/338; 514/357; 544/318; 514/274; 546/234; 435/8

(57) ABSTRACT

High throughput methods for identifying novel inhibitors of Hsp90 chaperone protein folding are disclosed. The inhibitors so identified disrupt the binding of p23 to either Hsp90α or Hsp90β and have selective activity against the proliferation of cancer cells. In particular are provided embodiments of therapeutic compositions that comprise at least one inhibitor of an Hsp90 chaperone activity, the inhibitor being any of the compounds designated as CP1-CP19 as shown in FIGS. 1A-1D or a 2-(trifluoromethyl)pyrimidin-2-yl)thio)acetamide derivatives.

### CROSS-REFERENCE TO RELATED APPLICATIONS

This application claims priority to U.S. Provisional patent application Ser. No. 61/649,560, entitled “MULTI-MODALITY MOLECULAR IMAGING HIGH-THROUGHPUT ASSAY FOR IDENTIFYING HEAT SHOCK PROTEIN 90 (HSP90) INHIBITORS” filed on May 21, 2012, and to U.S. Provisional Patent Application Ser. No. 61/663,763, entitled “MULTI-MODALITY MOLECULAR IMAGING HIGH-THROUGHPUT ASSAY FOR IDENTIFYING HEAT SHOCK PROTEIN 90 (HSP90) INHIBITORS” filed on Jun. 25, 2012, both of which are hereby incorporated by reference in their entireties.

### STATEMENT REGARDING FEDERALLY SPONSORED RESEARCH OR DEVELOPMENT

This invention was made with Government support under contracts CA082214 and CA114747 awarded by the National Institutes of Health. The Government has certain rights in this invention.

### TECHNICAL FIELD

The present disclosure is generally related to methods of identifying inhibitors of Hsp90 chaperone activity in a cell, in particular a cancer cell. The present disclosure is also generally related to the identified compounds and their use in inhibiting cancer cell growth.

### BACKGROUND

All proteins must be properly folded in order to perform their biological functions. In mammalian cells, the heat shock protein 90 (Hsp90) chaperone system, including Hsp90 and the co-chaperones p23, Hip, Hop and Hsp70 plays a key role in protein folding (Zhang & Burrows (2004) J. Mol. Med. 82: 488-499; Neckers & Ivy (2003) Curr. Opin. Oncol. 15: 419-424). Over-expression of Hsp90 in human cancers correlates with poor prognosis (Yano et al., (1999) Cancer Letts. 137: 45-51; Myung et al., (2004) Proteome Sci. 2: 8). The most important interaction within the Hsp90 chaperone system is between Hsp90(α/β) and p23, which occurs only when Hsp90 is bound to ATP. These interactions are crucial for maturation of functional Hsp90/client proteins complex, release of client proteins, transcription and signal transduction (Zhang & Burrows (2004) J. Mol. Med. 82: 488-499; Neckers & Ivy (2003) Curr. Opin. Oncol. 15: 419-424). Small molecule inhibitors with anti-tumor effects have been developed to inhibit Hsp90 ATPase activity by targeting its ATP binding pocket (Zhang & Burrows (2004) J. Mol. Med. 82: 488-499; Le Brazidec et al., (2004) J. Med. Chem. 47: 3865-3873; Chiosis, G., (2006) Current Top. Med. Chem. 6: 1183-1191). These inhibitors have higher binding affinities to Hsp90 in cancer compared to that of normal cells (Kamal et al., (2003) Nature 425: 407-410), and competitively block ATP binding to Hsp90 and subsequent binding of p23. The prevention of proper protein folding often leads to proteasome-mediated degradation of Hsp90 client proteins and simultaneous inhibition of multiple signal transduction pathways and cell growth arrest.

The geldanamycin (GA) analogue 17-(allylamino)-17-demethoxygeldanamycin (17-AAG) and 17-allylamino-17-demethoxygeldanamycin (17-DMAG) are currently in phase I/II clinical trials for treating advanced solid cancers (Jhaveri et al., (2012) Biochim. Biophys. Acta (Mol. Cell. Res.) 823: 742-755). A newer generation of geldanamycin-based Hsp90 inhibitors (Vetcher et al., (2005) Appl. Environ. Microbiol. 71:1829-835), as well as purine-scaffold (Vilenchik et al., (2004) Chem. Biol. 11: 787-797; Moulick et al., (2006) Bioorgan. Med. Chem. Letts. 16: 4515-4518; Llauger et al., (2005) J. Med. Chem. 48: 2892-2905; Immormino et al., (2006) J. Med. Chem. 49: 4953-4960; Chiosis et al., (2002) Bioorgan. Medicin. Chem. 10: 3555-3564; Chiosis et al., (2003) Curr. Cancer Drug Targets 3: 371-376; Chiosis, G. (2006) Exp. Opin. Therapeut. Targets, 10: 37-50), pyrazole-scaffold (McDonald et al., (2006) Curr. Top. Medicin. Chem. 6: 1193-1203), radicicol-based Hsp90 inhibitors (Soga et al., (2003) Curr. Cancer Drug Targets 3: 359-369; Proisy et al., (2006) Chem. Biol. 13: 1203-1215) and other compounds are currently in pre-clinical and/or Phase I/II clinical trials (Solit & Chiosis (2008) Drug Discovery Today 13: 38-43; Biamonte et al., (2009) J. Medicin. Chem. 53: 3-17). Despite the diversity of Hsp90 inhibitors reported, there is a constant need for developing novel Hsp90 inhibitors with better clinical therapeutic efficacy and reduced toxicity. The discovery of Hsp90 inhibitors achieved today was limited to in vitro analyses (Rowlands et al., (2004) Anal. Biochem. 327: 176-183; Soti et al., (2003) Eur. J. Biochem. 270: 2421-2428) and phenotypic assays aimed at examining the downstream effects of the inhibition of Hsp90/p23 interactions (Vilenchik et al., (2004) Chem. Biol. 11: 787-797; Chiosis et al., (2002) Bioorgan. Medicin. Chem. 10: 3555-3564; Chiosis et al., (2003) Curr. Cancer Drug Targets 3: 371-376; Soga et al., (2003) Curr. Cancer Drug Targets 3: 359-369). Even though small animal positron emission tomography (PET) (Smith-Jones et al., (2004) Nat. Biotechnol. 22: 701-706; Oude Munnink et al., (2010) Eur. J. Cancer 46: 678-684), magnetic resonance imaging (Ramirez et al., (2008) Am. J. Physiol. Renal Physiol. 295: F1044-1051) and ultrasound (Cao et al., (2008) Cancer Chemother. Pharmacol. 62: 985-994) have been used to monitor Her2 efficacy of Hsp90 inhibitors in mice, it was not possible to decipher the contribution of each Hsp90 isoform (α & β) in determining the sensitivity to Hsp90 inhibitors, since both isoforms are expressed in cancer cells, but play different roles in response to chemotherapy (Chang et al., (2006) Biochem Biophys Res Comms. 344: 37-44).

The efficacy of 11 different GA-based and purine-scaffold Hsp90 inhibitors in disruption of isoform-specific Hsp90(α/β)/p23 interactions has been monitored in intact cells in cell culture and in living mice using a genetically-encoded, split Renilla Luciferase (RL) complementation system (Chan et al., (2008) Cancer Res. 68: 216-226). This system is advantageous for developing novel Hsp90 inhibitors because 1) the potency of the Hsp90 inhibitors in disruption of signals from Hsp90(α/β)/p23 interactions correlates with their binding affinity for cellular Hsp90; 2) the interaction of each Hsp90 isoform (α/β) with p23 can be individually monitored; 3) RL does not require ATP for its activity, which facilitates the screening of inhibitors that target the ATP binding pocket of Hsp90; 4) the same reporter cells can be used for both high-throughput screening (HTS) in cell culture followed by dynamic monitoring of Hsp90(α/β)/p23 interactions in response to Hsp90 inhibitors in living mice; and 5) combination with other clinical imaging modalities such as PET allows monitoring of the downstream effects of Hsp90 inhibitors.

### SUMMARY

Briefly described, one aspect of the disclosure encompasses embodiments of a therapeutic composition comprising an inhibitor of an Hsp90 chaperone activity, wherein the inhibitor can be selected from the group consisting of: compounds CP1-CP19 as shown in FIGS. 1A-1D and a compound having the formula I:

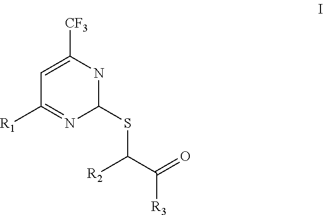

wherein R₁ can be a thiophene, a furan, a substituted or unsubstituted phenyl, or —OH; R₂ can be H or an alkyl; and R₃ can be phenylmethylamine, 4-amidopyridyl, or —NHR₄, wherein R₄ can be a substituted isoxazole, a substituted or unsubstituted alkyl, a substituted or unsubstituted branched chain alkyl, a substituted or unsubstituted —(CH)ₙPh, a substituted or unsubstituted 5 or 6-membered aryl, a substituted or unsubstituted 5 or 6-membered heteroaryl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine, and wherein the therapeutic composition can be formulated to have a dose of the inhibitor effective in reducing the viability of a cancer cell when delivered to an animal or human.

In embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In embodiments of this aspect of the disclosure, the inhibitor can have the formula I and can be selected from compounds CP9 and A1-A62 of FIGS. 2A-2G.

In some embodiments of this aspect of the disclosure, the inhibitor can have the formula I:

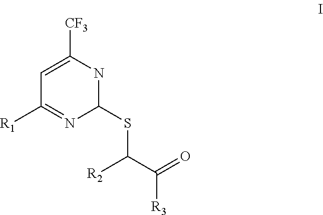

wherein R₁ can be a thiophene, a furan, phenyl, a substituted phenyl, or —OH; R₂ can be H or methyl; and R₃ can be phenylmethylamine, 4-amidopyridyl, or —NHR₄, wherein R₄ can be a substituted isoxazole, an alkyl, a branched chain alkyl, a —(CH)ₙPh, a substituted —(CH)ₙPh, -Ph, a substituted phenyl, a substituted biphenyl, a cycloalkyl, a pyridyl, —(CH)ₙpyridyl, methylfuranyl, a methyltetrahydrofuran; substituted pipenazyl, or a morpholine, and wherein n=1 or 2,
and the therapeutic composition can be formulated to have a dose of the inhibitor effective in reducing the viability of a cancer cell when delivered to an animal or human.

In these embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In these embodiments of this aspect of the disclosure, the inhibitor can be selected from compounds CP9 and A1-A62 of FIGS. 2A-2G.

In some embodiments of this aspect of the disclosure, the inhibitor is N-(5-methylisoxazol-3-yl)-2-(4-(thiophen-2-yl)-6-(trifluoromethyl)pyrimidin-2-ylthio)acetamide (CP9) having the formula:

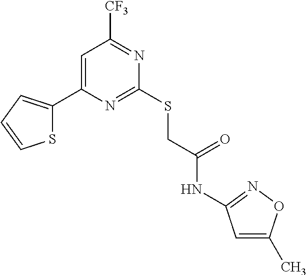 [SMILES: Cc1cc(NC(=O)CSc2nc(-c3cccs3)cc(C(F)(F)F)n2)no1]

In other embodiments of this aspect of the disclosure, the inhibitor can be CP9, A17, A29, or A61, or a combination thereof.

In the embodiments of this aspect of the disclosure, the therapeutic composition of the disclosure can further comprise a pharmaceutically acceptable carrier.

Another aspect of the disclosure encompasses embodiments of a method of reducing the viability of a cancer cell in an animal or human, the method comprising delivering to the animal or human a therapeutically effective amount of an inhibitor of an Hsp90 chaperone activity, wherein the inhibitor can be selected from the group consisting of: compounds CP1-CP19 as shown in FIGS. 1A-1D and a compound having the formula I:

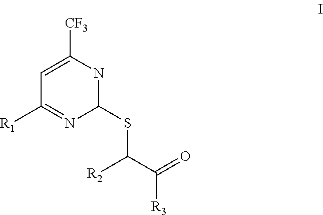

wherein R₁ can be a thiophene, a furan, a substituted or unsubstituted phenyl, or —OH; R₂ can be H or an alkyl; and R₃ can be phenylmethylamine, 4-amidopyridyl, or —NHR₄, wherein R₄ can be a substituted isoxazole, a substituted or unsubstituted alkyl, a substituted or unsubstituted branched chain alkyl, a substituted or unsubstituted —(CH)ₙPh, a substituted or unsubstituted 5 or 6-membered aryl, a substituted or unsubstituted 5 or 6-membered heteroaryl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine.

In embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In embodiments of this aspect of the disclosure, the inhibitor can have the formula I and can be selected from compounds CP9 and A1-A62 of FIGS. 2A-2G.

In embodiments of this aspect of the disclosure, the inhibitor can have the formula I:

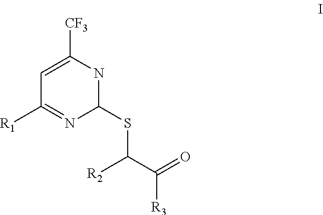

wherein R₁ can be a thiophene, a furan, phenyl, a substituted phenyl, or —OH; R₂ can be H or methyl; and R₃ can be phenylmethylamine, 4-amidopyridyl, or —NHR₄, wherein R₄ can be a substituted isoxazole, an alkyl, a branched chain alkyl, a —(CH)ₙPh, a substituted —(CH)ₙPh, -Ph, a substituted phenyl, a substituted biphenyl, a cycloalkyl, a pyridyl, —(CH)ₙpyridyl, methylfuranyl, a methyltetrahydrofuran, substituted pipenazyl, or a morpholine, and wherein n=1 or 2, and where the therapeutic composition can be formulated to have a dose of the inhibitor effective in reducing the viability of a cancer cell when delivered to an animal or human.

In these embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In some embodiments of this aspect of the disclosure, the inhibitor can be selected from compounds CP9 and A1-A62 of FIGS. 2A-2G.

In some embodiments of this aspect of the disclosure, the inhibitor is N-(5-methylisoxazol-3-yl)-2-(4-(thiophen-2-yl)-6-(trifluoromethyl)pyrimidin-2-ylthio)acetamide (CP9) having the formula:

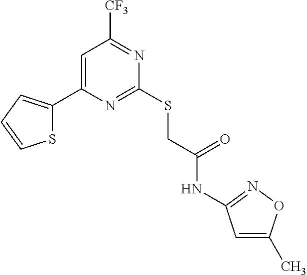 [SMILES: Cc1cc(NC(=O)CSc2nc(-c3cccs3)cc(C(F)(F)F)n2)no1]

In some embodiments of this aspect of the disclosure, the inhibitor can be CP9, A17, A29, or A61, or a combination thereof.

Yet another aspect of the disclosure encompasses embodiments of a high-throughput method for identifying an inhibitor of Heat Shock Protein 90 (Hsp90) chaperone activity, the system comprising: (a) obtaining a genetically modified cell, or progeny thereof expressing a split Renilla luciferase reporter configured to provide a detectable signal on binding of a p23 polypeptide and a Heat Shock Protein 90 (Hsp90) polypeptide in the presence of coelentarazine; (b) detecting a first detectable signal emitted from the genetically-modified cell or population thereof; (c) contacting the genetically-modified cell or progeny thereof with a compound suspected of being an Hsp90 inhibitor; (d) detecting a second detectable signal emitted from the genetically-modified cell or progeny thereof expressing the split Renilla luciferase reporter; and (e) comparing the intensities of the first and the second detectable signals, whereby if the intensity of the first detectable signal is greater than intensity of the second detectable signal, the compound is determined to inhibit the formation of a complex between p23 and an Hsp90 polypeptide.

In some embodiments of this aspect of the disclosure, the method can further comprise the steps: (f) obtaining a subject animal comprising a xenograft tumor derived from the genetically-modified cell of step (a); (g) administering to the animal coelentarazine and detecting a third detectable signal intensity from the xenograft tumor; and (h) administering to the subject animal the compound determined in step (e) to inhibit complex formation between p23 and an Hsp90 polypeptide, and coelentarazine, and obtaining a fourth detectable signal intensity from the xenograft, wherein if the fourth signal intensity is less than the third signal intensity, the compound identified in step (e) is identified as an inhibitor of complex formation between p23 and an Hsp90 polypeptide in vivo.

In some embodiments of this aspect of the disclosure, the split luciferase reporter comprises a p23 polypeptide having an N-terminus fragment of a Renilla luciferase attached thereto, and an Hsp90 polypeptide having a C-terminus fragment of the Renilla luciferase attached thereto, whereby when the p23 and the Hsp90 polypeptides are in contact in the presence of ATP the N- and C-termini of the Renilla luciferase cooperate to generate the first detectable signal in the presence of coelentarazine.

Still another aspect of the disclosure encompasses embodiments of a kit comprising a container containing a therapeutic composition comprising a compound of FIGS. 1A-1D and 2A-2G, or a pharmaceutically effective derivative thereof, and instructions for administering the compounds or formulations to a subject.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1D illustrate the structural formulae of compounds C1-C19 identified from the combinatorial library 1280 LOPAC.

FIGS. 2A-2G illustrate the structural variants of the structure having formula I (FIG. 2A) (2-{[6-(trifluoromethyl)pyrimidin-2-yl]thio}acetamide.

FIG. 3 schematically illustrates the monitoring of Hsp90/p23 interaction using a split Renilla luciferase (RL) complementation system. The two interacting proteins p23 and Hsp90 are fused to the inactive ‘N-RL’ and ‘C-RL’ portions of the RL through a peptide linker (top right). In the presence of ATP, interactions between p23/Hsp90 bring ‘N-RL’ and ‘C-RL’ in close proximity and lead to the complementation of RL enzyme activity and photon production (hυ) in the presence of the substrate coelentarazine. Binding of Hsp90 inhibitors (I) to Hsp90-CRL leads to a conformation change and prevents ATP from binding, thus diminishing the interaction between NRL-p23 and Hsp90-CRL to reduce light output.

FIG. 4A illustrates a dose-dependent decrease in bioluminescence signals in 293T cells stably expressing Hsp90α/p23 and Hsp90β/p23 split RL reporters treated with compound CP1 at the indicated dose for 24 h (left). Bioluminescence signals were quantified and normalized for cell number and that of carrier control treated cells (right). Results were expressed as mean±S.E.M.

FIG. 4B illustrates the dose-dependent decrease in bioluminescence signals in 293T cells stably expressing Hsp90α/p23 and Hsp90β/p23 split RL reporters treated with compound CP9 for 24 h. Results were expressed as mean±S.E.M.

FIG. 4C illustrates the dose-dependent decrease in bioluminescence signals in 293T cells stably expressing Hsp90α/p23 and Hsp90β/p23 split RL reporters treated with compound CP18 for 24 h. Results were expressed as mean±S.E.M.

FIGS. 5A-5D illustrate the validation of the mechanisms of lead compounds as Hsp90 inhibitors.

FIG. 5A is a digital image of a western blot analysis illustrating the effect of lead compounds on the expression of Hsp90 client proteins. 293T cells stably expressing Hsp90(α/β)/p23 split reporters were treated with carrier control or compounds CP1, CP9, or CP18 (5 μM each) for 24 h. The known Hsp90 inhibitor PU-H71 (2 μM) was used as a positive control. The expression of Hsp90 client proteins (Raf-1, total and phosphorylated Akt) was determined by western blotting. α-Tubulin was used as a loading control.

FIG. 5B is a digital image of a western blot analysis illustrating the effects of compounds CP1, CP9 and CP18 on disruption of Hsp90α/p23 and Hsp90β/p23 as determined by co-immunoprecipitation using antibodies against p23 and probing with Hsp90α- and Hsp90β-specific antibodies. The blot was probed with antibodies against p23 to ensure similar immunoprecipitation efficiency. Cells treated with 2 μM of PU-H71 served as a positive control.

FIG. 5C is a graph illustrating the competitive in vitro binding of CP9 with ³H-17AAG to Hsp90(α/β). Purified Hsp90(α/β) was pre-incubated with CP9 (200 μM) or cold 17-AAG (50 μM) prior to incubation with ³H-17AAG. The reaction complexes were then eluted through a desalting column to remove any unbound ³H-17AAG prior to scintillation counting for the amount of ³H-17AAG bound to Hsp90(α/β). Columns incubated with ³H-17AAG alone served as negative controls. Results are expressed as means of triplicates±S.E.M. *, p<5×10⁻⁶ vs. carrier control-treated cells.

FIG. 5D is a graph illustrating the inhibition of ³H-17AAG uptake by CP9 in HT29 cells. HT29 cells were pre-incubated with different concentrations of CP9 together with ³H-17AAG (0.5 μM) for 1 h followed by 1 h of wash-out of unbound ³H-17AAG in fresh medium. The amount of ³H-17-AAG incorporated intracellularly was determined by scintillation counting of cell lysates, followed by normalization for protein content and to carrier control-treated cells. Cells incubated with 5 μM of PU-H71 served as a positive control. Results are expressed as means of triplicates±S.E.M. *, p<0.05 vs. carrier control-treated cells.

FIGS. 6A and 6B illustrate the effect of CP9 on glucose metabolism and cell proliferation in different cancer cell lines.

FIG. 6A is a series of graphs illustrating the effect of CP9 on cell proliferation determined by an Alamar Blue assay. The indicated cancer cell lines were treated with different concentrations of CP9 for 24 h prior to incubation with Alamar blue for 4 h. The Hsp90 inhibitors PU-H71 and 17AAG were used as positive controls. Emissions at 570 nm in CP9-treated cells were normalized to that of carrier control-treated cells. Results are expressed as normalized cell proliferation±S.E.M.

FIG. 6B is a graph illustrating the effect of CP9 on glucose metabolism determined in 1975 lung cancer, 2008 ovarian cancer and HT29 colon cancer cells by ³H-FDG cell uptake studies 24 h post treatment. Normal MEFs were used as a control. Results were expressed as mean counts/μg protein/initial dose±S.E.M. *, p<0.05 vs. carrier control treated cells.

FIGS. 7A and 7B are digital images illustrating bioluminescence imaging of the efficacy of CP9 in disruption of Hsp90(α/β)/p23 interactions and cell proliferation. Nude mice bearing 293T xenografts expressing the Hsp90(α/β)/p23 split RL reporters and eGFP-FL reporters (293T(α/β)/p23-FG) were used. Mice were imaged for baseline RL signals from Hsp90α/p23 (left tumors in each animal) and Hsp90β/p23 interactions (right tumors) using a cooled CCD camera upon i.v. injection of coelentarazine (cltz). The RL signals were allowed to decay for 30 min to allow clearance of the coelentetrazine before monitoring the baseline cell proliferation by FL imaging upon i.p. injection of D-Luciferin (D-luc). Mice (N=5 per group) were then randomized based on FL signals for treatment with CP9 (80 mg/kg in DMSO×4 doses), or carrier control (DMSO) by i.v. injections. Mice (N=2) treated with PU-H71 (75 mg/kg) served as positive controls. RL and FL signals were determined at different time points post-treatment upon re-injection of cltz and D-Luc. Representative RL (left panel) and FL images (right panel) from mice treated with CP9 (FIG. 7A) or carrier control (FIG. 7B) are shown.

FIG. 7C shows a pair of graphs illustrating the quantitation of RL and FL signals for each mouse performed by drawing the same size ROI on each site at all time points. Maximum radiance (photons/sec/cm²/sr) from RL signals was normalized to that of FL signals at each site and at each time point to account for the effect of CP9 on cell proliferation. The RL/FL ratios were normalized to that of time 0 h to monitor the individual change in Hsp90(α/β)/p23 interactions in each mouse over time. Results were expressed as mean±S.E.M for the control and treatment groups for Hsp90α/p23 (left panel) and Hsp90β/p23 interactions (right panel).

FIGS. 8A-8C illustrate that CP9 inhibits glucose metabolism in tumor xenografts by PET/CT imaging but does not significantly degrade Hsp90 client proteins.

FIG. 8A shows digital images illustrating the downstream effects of CP9 on glucose metabolism in 293T Hsp90(α/β)/p23-FG xenografts using small animal PET/CT imaging and ¹⁸F-FDG. Mice were imaged using the CT module of the PET/CT machine prior to PET scanning. Baseline ¹⁸F-FDG uptake was determined at time 0 prior to treatment with four doses of CP9 (N=5 per group) or carrier control (N=4 per group). Mice were re-imaged at 43 h. PET, CT and maximum intensity projections of the PET/CT images of representative mice treated with CP9 (left panels) or carrier control (right panels) are shown. The dotted circles outline the locations of the Hsp90α/p23 (left flanks) and Hsp90β/p23 xenografts (right flanks). The side bar represents % ID/g for ¹⁸F-FDG uptake in tumors in PET images.

FIG. 8B is a graph illustrating quantitation of the effect of CP9 on inhibition of glucose metabolism. The PET image of each mouse and each site at each time point was reconstructed and quantified using the OSEM 2D algorithm. The increase in max % ID/g at 43 h relative to 0 hr is shown as average±S.E.M. CP9 led to significant decrease in glucose metabolism, compared to carrier control treated mice (p<0.005).

FIG. 8C is a digital image of a western blot analysis illustrating the validation of the mechanism of CP9 as an Hsp90 inhibitor in living mice. Tumors were excised from the mice and the expression of Raf-1, phosphorylated and total Akt determined by western blotting. α-Tubulin was used as a loading control. CP9 did not led to a significant decrease in the expression of Hsp90 client proteins, relative to carrier control treated mice.

FIG. 9A is a graph illustrating the results of high-throughput screening (HTS) of the 1280-Library of Pharmacological Active Compounds (LOPAC1280®, Sigma-Aldrich Inc.) to identify novel inhibitors of Hsp90(α/β)/p23 interactions in intact 293T cells. 293T cells stably expressing the Hsp90α/p23 (solid bars) or Hsp90β/p23 (striped bars) split RL reporter constructs were plated in 384-well white plates for 24 hours. Baseline RL signals were determined by a luminescence reader upon addition of the RL substrate ENDUREN®. Each compound in the LOPAC library (20 μM) or carrier control was added to each well and RL signals at 24 h post treatment were normalized to that of time 0 and carrier controls. The experiment was repeated twice and the % inhibition of RL activity for the top 20 compounds shown as mean±S.E.M

FIG. 9B is a schematic flow diagram for screening of a 3×10⁴ small molecule compound library. Initial screening was performed as described in the legend to FIG. 9A, above, except only one well was used for each compound at 20 μM. IC₅₀ values for the compounds that led to greater than 45% inhibition of Hsp90α/p23 or Hsp90β/p23 interactions were determined by a 7-point dose response experiment (0.3-20 μM). Toxic compounds (determined by a CELL TITER BLUE ASSAY®) were eliminated. The number within the parenthesis denotes the number of compounds at each stage during the screening.

FIG. 9C is a series of digital images illustrating the elimination of non-specific RL inhibitors and validation of the lead compounds from HTS. The 9 compounds as used in the generation of the data of FIG. 9A were tested in cell culture in quadruplicate wells to eliminate compounds that non-specifically inhibit RL activities. 293T cells stably expressing full length RL were treated with carrier controls (Rows D-H, columns 9-12) or 10-fold higher concentration of the compounds (CP1-19) at their respective IC₅₀ values for inhibition of Hsp90α/p23 interactions. RL activities in intact cells were determined by BLI at different time points post-treatment using a cooled-CCD camera upon addition of ENDUREN®.

A1-4: CP1; B1-4: CP2; C1-4: CP3; D1-4: CP4; E1-4: CP5; F1-4: CP6; G1-4: CP7; H1-5: CP8; A5-8: CP9; B5-8: CP10; C5-8: CP11; D5-8: CP12; E5-8: CP13; F5-8: CP14; G5-8: CP15; H5-8: CP16; A9-12: CP17; B9-12: CP18; C9-12: CP19; D-H9-12: carrier control.

FIG. 9D is a graph illustrating the quantitation of the bioluminescence signals of FIG. 9C. The average radiance for the quadruplicate wells were averaged and normalized to that of cells treated with carrier-controls. Results were expressed as mean±S.E.M. Compounds that non-specifically inhibit RL activities (>20% vs. carrier control treated cells) were subsequently eliminated.

FIGS. 10A-10D illustrate the degradation of multiple Hsp90 client proteins by CP9 in different cancer cell lines.

FIGS. 10A and 10B show the effect of CP9 on the expression of Raf-1, phosphorylated Akt, and total Akt in 1975 lung cancer, BT474 breast cancer and HuH-7 liver cancer as determined by western blotting. α-Tubulin was used as the loading control.

FIG. 10C shows the effect of CP9 on expression of Hsp90 client proteins in normal mouse embryonic fibroblast cells (MEF). CP9 did not significantly decrease the level of Hsp90 client proteins in MEFs at the indicated doses.

FIG. 10D is a graph illustrating the effect of CP9 on mammalian thymidine kinase activities in 1975, 2008, HT29, and MEFs cells as determined by ³H-FLT cell uptake studies 48 h post treatment. Results were expressed as mean counts per minute/μg protein/initial dose±S.E.M. *, p<0.05 vs. carrier control treated cells.

FIGS. 11A-11F illustrate the evaluation of CP9 analogues in disruption of Hsp90(α/β)/p23 interactions in 293T cells. 293T cells stably expressing Hsp90(α/β)/p23 split RL reporters were treated with CP9 (0.63 to 10 μM) or its 63 different structural analogues (10 μM) or carrier control for 24 h. Duplicate wells were used for each analogue. BLI was performed with a cooled CCD camera. RL imaging was performed 2 h post addition of ENDUREN®.

FIG. 11A shows RL imaging of the inhibition of Hps90α/p23 interactions by CP9 and its analogues. A 6-point dose-response curve was established for CP9 (Rows A-F, Columns 1-3). Duplicate wells were used for each analogue concentration. PU-H71 and 17AAG were used as positive controls at 10 μM in triplicates (Rows C-F/Column 12). Cells treated with 1% DMSO served as carrier controls (Rows G/H, Columns 1-5).

FIG. 11B is a graph illustrating that CP9 and its analogues led to different levels of inhibition of Hsp90(α/β)/p23 interactions. RL signals were normalized to that of Alamar Blue signals, followed by normalization to that of carrier control treated cells as mean±S.E.M. The numbers next to each diamond (Hsp90α/p23) and square (Hsp90β/p23) denote the analogue number (A1-A31). The dotted lines denote the level of inhibition of Hsp90(α/β)/p23 interactions by the parental compound CP9 at 10 μM.

FIG. 11C is a graph illustrating the time- and dose-dependent decreases in RL signals in 293Tα-FG cells by CP9 and its four most potent analogues. Stable cells expressing the Hsp90(α/β)/p23 split RL reporters and eGFP-FL fusion proteins were treated with indicated concentrations of CP9, its analogues or carrier control prior to BLI of RL signals upon addition of ENDUREN®. RL signals were normalized to that of carrier control treated cells and expressed as mean±S.E.M.

FIG. 11D is a graph illustrating the time- and dose-dependent decreases in RL signals in 293Tβ-FG cells by CP9 and its four most potent analogues.

FIGS. 11E and 11F are graphs illustrating dose-dependent decreases in Hsp90(α/β)/p23 interactions by CP9 and its analogues A17, A29, and A48 monitored by 293Tα-FG and 293Tβ-FG cells. PU-H71 and 17-AAG were used as positive controls. 293T stable cells were treated with indicated concentrations of CP9, its analogues or carrier control for 24 hours. Disruption of Hsp90(α/β)/p23 interactions and inhibition of cell proliferation were monitored by RL and FL imaging respectively. RL signals were normalized to that of FL signals and to carrier control treated cells, as expressed as mean±S.E.M.

FIG. 11G is a graph illustrating dose-dependent decreases in Hsp90 client proteins Raf-1 and phosphorylated and total Akt in 293Tα/p23-FG cells by CP9, its analogues A17 and A29 as determined by western blotting. Cells treated with PU-H71 and 17-AAG (2.5 μM) served as positive controls. α-Tubulin was used as the loading control.

FIGS. 12A-12D illustrate the effects of compound A17 on cell proliferation and in living mice.

FIG. 12A is a graph illustrating the effect of A17, CP9 and 17-AAG on proliferation of 293T cells. 293T cells were treated with different concentrations of inhibitors in triplicates for 24 h prior to determination of cell proliferation by Alamar Blue assay. Emission signals were normalized to that of carrier control treated cells and expressed as mean±S.E.M.

FIG. 12B is a graph illustrating the effect of compound A17 on Hsp90α/p23 interactions in mice bearing 293T xenografts. Mice were treated with 4 doses of A17 (80 mg/kg) post establishment of baseline RL signals (Hsp90α/p23) and FL signals (cell proliferation), and re-imaged at the indicated time points. Relative to carrier control mice, A17 led to decreases in Hsp90 α/p23 interactions at 43 h but of limited statistical significance (p>0.05).

FIG. 12C is a graph illustrating the effect of A17 on Hsp90β/p23 interactions in mice bearing 293T xenografts. A17 did not lead to significant decrease in Hsp90β/p23 interactions (p >0.05 relative to carrier control mice).

FIG. 12D is a graph illustrating ex-vivo analyses of Hsp90 client proteins in excised tumors. The expression of phosphorylated and total Akt and Raf-1 was determined by western blotting. α-Tubulin was used as the loading control. A17 did not lead to significant decreases in Hsp90 client proteins (p>0.05 relative to carrier control mice).

FIGS. 13A-13C illustrate the inhibition of cell proliferation of DOX-resistant V79/ADR lung cancer cells by CP9 and the Hsp90 inhibitor PU-H71 in cell culture. V79 lung cancer cells and V69/ADR cells that are resistant to DOX were treated with DOX (0.31-10 μM) in the presence or absence of CP9 (8 μM) for 24 h before determination of cell number by an Alamar Blue assay.

FIG. 13A is a graph showing that CP9 did not affect the sensitivity of drug-sensitive V79 cells to DOX.

FIG. 13B is a graph showing that CP9 partially restored the sensitivity of drug-resistant V79/ADR cells to DOX.

FIG. 13C is a graph showing that PU-H71 restored the sensitivity of drug-resistant V79/ADR cells to DOX.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### Abbreviations

HTS, high throughput screening; RL, Renilla luciferase; FL, firefly luciferase; cltz, coelentarazine; GA, geldanamycin; 17-AAG, 17-(allylamino)-17-demethoxygeldanamycin; 17-DMAG, 17-allylamino-17-demethoxygeldanamycin; Hsp, heat shock protein; PET, positron emission tomography; CT, computed tomography; MEF, mouse embryonic fibroblast cells; ³H-FDG, ³H-fluoro-deoxyglucose.

### DEFINITIONS

In describing and claiming the disclosed subject matter, the following terminology will be used in accordance with the definitions set forth below.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a”, “an,” and “the” include plural referents unless the content clearly dictates otherwise. Thus, for example, reference to a composition comprising “a compound” includes a mixture of two or more compounds.

The terms “administering” and “administration” as used herein refer to a process by which a therapeutically effective amount of a compound of the disclosure or compositions contemplated herein are delivered to a subject for prevention and/or treatment purposes. Compositions are administered in accordance with good medical practices taking into account the subject's clinical condition, the site and method of administration, dosage, patient age, sex, body weight, and other factors known to physicians.

The terms “co-administration” of “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s). The term “treating” as used herein refers to reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present disclosure to a subject that is not at the time of administration afflicted with the disease. “Preventing” also refers to preventing the recurrence of a disease or of one or more symptoms associated with such disease. “Treatment” and “therapeutically” refer to the act of treating, as “treating” is defined above. The purpose of prevention and intervention is to combat the disease, condition, or disorder and includes the administration of an active compound to prevent or delay the onset of the symptoms or complications, or alleviating the symptoms or complications, or eliminating the disease, condition, or disorder.

The terms “subject,” “individual,” or “patient” as used herein are used interchangeably and refer to an animal, preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g., gorillas or chimpanzees), and rodents such as rats and mice.

Typical subjects for treatment include persons afflicted with or suspected of having or being pre-disposed to a disease disclosed herein, or persons susceptible to suffering from or that have suffered from a disease disclosed herein. A subject may or may not have a genetic predisposition for a disease disclosed herein. In the context of certain aspects of the disclosure, the term “subject” generally refers to an individual who will receive or who has received treatment (e.g., administration of a compound of the disclosure, and optionally one or more other agents) for a condition characterized by a cancer. In certain aspects, a subject may be a healthy subject.

The term “healthy subject” means a subject, in particular a mammal, having no diagnosed disease, disorder, infirmity, or ailment, more particularly a disease, disorder, infirmity or ailment known to impair or otherwise diminish memory.

The term “diagnosed,” as used herein, refers to the recognition of a disease by its signs and symptoms (e.g., resistance to conventional therapies), or genetic analysis, pathological analysis, histological analysis, and the like.

The term “modulate” refers to the activity of a composition affecting (e.g., to promote or retard) an aspect of cellular function, including, but not limited to, cell growth, proliferation, apoptosis, and the like.

The term “therapeutically effective amount” relates to the amount or dose of an active compound of the disclosure or composition comprising the same that will lead to one or more desired effects, in particular, one or more therapeutic effects or beneficial pharmacokinetic profiles. A therapeutically effective amount of a substance can vary according to factors such as the disease state, age, sex, and weight of the subject and the ability of the substance to elicit a desired response in the subject. A dosage regimen may be adjusted to provide the optimum therapeutic response or pharmacokinetic profile. For example, several divided doses may be administered daily or the dose may be proportionally reduced as indicated by the exigencies of the therapeutic situation.

The term “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “beneficial pharmacokinetic profile” refers to amounts or doses of a compound of the disclosure that provide levels of the compound or a required dose resulting in therapeutic effects in the prevention, treatment, or control of symptoms of a disease disclosed herein. The term “sustained pharmacokinetic profile” as used herein refers to a length of time efficacious levels of a biologically active compound of the disclosure is in its environment of use. A sustained pharmacokinetic profile can be such that a single or twice-daily administration adequately prevents, treats, or controls symptoms of a disease disclosed herein. A beneficial pharmacokinetic profile may, but is not limited to, providing therapeutically effective amounts of the compound of the disclosure in the subject for about 12 h to about 48 h, 12 h to about 36 h, or 12 h to about 24 h.

The term “therapeutic effect” as used herein refers to an effect of a composition of the disclosure, in particular a formulation or dosage form, or method disclosed herein. A therapeutic effect may be a sustained therapeutic effect that correlates with a continuous concentration of a compound of the disclosure over a dosing period, in particular a sustained dosing period. A therapeutic effect may be a statistically significant effect in terms of statistical analysis of an effect of a compound of the disclosure versus the effects without the compound.

“Statistically significant” or “significantly different” effects or levels may represent levels that are higher or lower than a standard. In aspects of the disclosure, the difference may be 1.5, 2, 3, 4, 5, 6, 7, 8, 9, 10, 15, 20, 25 or 50 times higher or lower compared with the effect obtained without a compound of the disclosure.

The term “pharmaceutically acceptable carrier” as used herein refers to a diluent, adjuvant, excipient, or vehicle with which a therapeutic composition according to the disclosure is administered and which is approved by a regulatory agency of the federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like.

A composition of the disclosure may include a carrier. Suitable carriers include a polymer, carbohydrate, or a peptide. A “polymer” refers to molecules comprising two or more monomer subunits that may be identical repeating subunits or different repeating subunits. A monomer generally comprises a simple structure such as a low-molecular weight molecule containing carbon. Polymers may optionally be substituted. Polymers that can be used in the present disclosure include without limitation vinyl, acryl, styrene, carbohydrate-derived polymers, polyethylene glycol (PEG), polyoxyethylene, polymethylene glycol, poly-trimethylene glycols, polyvinylpyrrolidone, polyoxyethylene, polyoxypropylene block polymers, and copolymers, salts, and derivatives thereof. In aspects of the disclosure, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl,-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and polyvinyl alcohol.

The term “carbohydrate” as used herein refers to a polyhydroxyaldehyde, polyhydroxyketone and derivatives thereof. The term includes monosaccharides such as erythrose, arabinose, allose, altrose, glucose, mannose, threose, xylose, gulose, idose, galactose, talose, aldohexose, fructose, ketohexose, ribose, and aldopentose. The term also includes carbohydrates composed of monosaccharide units, including disaccharides, oligosaceharides, or polysaccharides. Examples of disaccharides are sucrose, lactose, and maltose. Oligosaccharides generally contain between 3 and 9 monosaccharide units and polysaccharides contain greater than 10 monosaccharide units. A carbohydrate group may be substituted at one, two, three or four positions, other than the position of linkage to a compound of the disclosure. For example, a carbohydrate may be substituted with one or more alkyl, amino, nitro, halo, thiol, carboxyl, or hydroxyl groups, which are optionally substituted. Illustrative substituted carbohydrates are glucosamine, or galactosamine. In aspects of the disclosure, the carbohydrate is a sugar, in particular a hexose or pentose and may be an aldose or a ketose. A sugar may be a member of the D or L series and can include amino sugars, deoxy sugars, and their uronic acid derivatives. In embodiments of the disclosure where the carbohydrate is a hexose, the hexose is glucose, galactose, or mannose, or substituted hexose sugar residues such as an amino sugar residue such as hexosamine, galactosamine, glucosamine, in particular D-glucosamine (2-amino-2-doexy-D-gluoose) or D-galactosamine (2-amino-2-deoxy-D-galactose). Illustrative pentose sugars include arabinose, fucose, and ribose. A sugar residue may be linked to a compound of the disclosure from a 1,1 linkage, 1,2 linkage, 1,3 linkage, 1,4 linkage, 1,5 linkage, or 1,6 linkage. A linkage may be via an oxygen atom of a compound of the disclosure. An oxygen atom can be replaced one or more times by —CH₂— or —S— groups. The term “carbohydrate” also includes glycoproteins such as lectins (e.g., concanavalin A, wheat germ agglutinin, peanutagglutinin, seromucoid, and orosomucoid) and glycolipids such as cerebroside and ganglioside.

A “peptide” carrier for use in the practice of the present disclosure includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. A carrier for use in the present disclosure can be an amino acid such as alanine, glycine, praline, methionine, serine, threonine, histidine, asparagine, alanyl-alanyl, prolyl-methionyl, or glycyl-glycyl. A carrier can be a polypeptide such as albumin, antitrypsin, macroglobulin, haptoglobin, cacruloplasm, transferring, .alpha.- or .beta.-lipoprotein, .beta.- or .gamma.-globulin or fibrinogen.

Approaches to designing peptide analogues, derivatives and mimetics are known in the art. For example, see Farmer, P. S. in Drug Design (E. Ariens, ed.) Academic Press, New York, 1980, vol. 10, pp. 119-143; Ball & Alewood (1990) J. Mol. Recognition. 3: 55; Morgan & Gainor, (1989) Ann. Rep. Med. Chem. 24: 243; and Freidinger, R. M. (1989) Trends Pharmacol. Sci. 10: 270. See also Sawyer, T. K. (1995) “Peptidomimetic Design and Chemical Approaches to Peptide Metabolism” in Taylor, M. D. & Amidon, G. L. (eds.) Peptide-Based Drug Design: Controlling Transport and Metabolism, Chapter 17; Smith et al, (1995) J. Am. Chem. Soc. 117: 11113-11123; Smith et al. (1994) J. Am. Chem. Soc. 116: 9947-9962; and Hirschman et al. (1993) Am. Chem. Soc. 115: 12550-12568.

A peptide can be attached to a compound of the disclosure through a functional group on the side chain of certain amino acids (e.g., serine) or other suitable functional groups. A carrier may comprise four or more amino acids with groups attached to three or more of the amino acids through functional groups on side chains. In an aspect, the carrier is one amino acid, in particular a sulfonate derivative of an amino acid, for example cysteic acid.

A compound of the disclosure can contain one or more asymmetric centers and may give rise to enantiomers, diasteriomers, and other stereoisomeric forms which may be defined in terms of absolute stereochemistry as (R)- or (S)-. Thus, compounds of the disclosure include all possible diasteriomers and enantiomers as well as their racemic and optically pure forms. Optically active (R)- and (S)-isomers may be prepared using chiral synthons or chiral reagents, or resolved using conventional techniques. When a compound of the disclosure contains centers of geometric asymmetry, and unless specified otherwise, it is intended that the compounds include both E and A geometric isomers. All tautomeric forms are also included within the scope of a compound of the disclosure.

A compound of the disclosure includes crystalline forms which may exist as polymorphs. Solvates of the compounds formed with water or common organic solvents are also intended to be encompassed within the term. In addition, hydrate forms of the compounds and their salts are encompassed within this disclosure. Further prodrugs of compounds of the disclosure are encompassed within the term.

The term “solvate” means a physical association of a compound with one or more solvent molecules or a complex of variable stoichiometry formed by a solute (for example, a compound of the disclosure) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like. Dehydrate, co-crystals, anhydrous, or amorphous forms of the compounds of the disclosure are also included. The term “hydrate” means a solvate wherein the solvent molecule(s) is/are H₂O, including mono-, di-, and various poly-hydrates thereof. Solvates can be formed using various methods known in the art.

Crystalline compounds of the disclosure can be in the form of a free base, a salt, or a co-crystal. Free base compounds can be crystallized in the presence of an appropriate solvent in order to form a solvate. Acid salt compounds of the disclosure (e.g., HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

The amount of solvent used to make solvates can be determined by routine testing. For example, a monohydrate of a compound of the disclosure would have about 1 equivalent of solvent (H₂O) for each equivalent of a compound of the disclosure. However, more or less solvent may be used depending on the choice of solvate desired.

Compounds of the disclosure may be amorphous or may have different crystalline polymorphs, possibly existing in different salvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

The term “prodrug” as used herein means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen & Bundgaard (eds.), Gordon & Breach, 1991, particularly Chapter 5: “Design and Applications of Prodrugs”; Design of Prodrugs, Bundgaard (ed.), Elsevier, 1985; Prodrugs: Topical and Ocular Drug Delivery, K. B. Sloan (ed.), Marcel Dekker, 1998; Widder et al. Methods in Enzymol., (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, Higuchi & Stella (eds.), Am. Chem. Soc., 1975; and Bioreversible Carriers in Drug Design, E. B. Roche (ed.), Elsevier, 1987. Examples of prodrugs include, but are not limited to, esters (e.g., acetate, formate, and benzoate derivatives), carbamates (e.g., N,N-dimethylaminocarbonyl) of hydroxy functional groups on compounds of the disclosure, and the like

A compound of the disclosure can include a pharmaceutically acceptable co-crystal or a co-crystal salt. A pharmaceutically acceptable co-crystal includes a co-crystal that is suitable for use in contact with the tissues of a subject or patient without undue toxicity, irritation, allergic response and has the desired pharmacokinetic properties.

The term “co-crystal” as used herein means a crystalline material comprised of two or more unique solids at room temperature, each containing distinctive physical characteristics, such as structure, melting point, and heats of fusion. Co-crystals can be formed by an active pharmaceutical ingredient (API) and a co-crystal former either by hydrogen bonding or other non-covalent interactions, such as pi stacking and van der Waals interactions. An aspect of the disclosure provides for a co-crystal wherein the co-crystal former is a second API. The co-crystal former need not be an API. The co-crystal can comprise more than one co-crystal former. For example, two, three, four, five, or more co-crystal formers can be incorporated in a co-crystal with an API. Pharmaceutically acceptable co-crystals, are described, for example, in “Pharmaceutical co-crystals,” Journal of Pharmaceutical Sciences, Volume 95 (3) Pages 499-516, 2006.

A co-crystal former, which is generally a pharmaceutically acceptable compound, may be, for example, benzoquinone, terephthalaldehyde, saccharin, nicotinanaide, acetic acid, formic acid, butyric acid, trimesic acid, 5-nittoisophthalic acid, adamantane-1,3,5,7-tetracarboxylic acid, formamide, succinic acid, fumaric acid, tartaric acid, oxalic acid, tartaric acid, malonic acid, benzamide, mandelic acid, glycolic acid, fumaric acid, maleic acid, urea, nicotinic acid, piperazine, p-phthalaldehyde, 2,6-pyridinecarboxylic acid, 5-nitroisophthalic acid, citric acid, and the alkane- and arene-sulfonic acids such as methanesulfonic acid and benzenesulfonic acid. In general, all physical forms of compounds of the disclosure are intended to be within the scope of the present disclosure.

A compound of the disclosure may be pure or substantially pure. As used herein, the term “pure” in general means better than 90%, 92%, 93%, 94%, 95%, 96%, 97%, 98% or 99% pure, and “substantially pure” means a compound synthesized such that the compound, as made or as available for consideration into a composition or therapeutic dosage described herein, has only those impurities that may not readily nor reasonably be removed by conventional purification processes.

A compound of the disclosure includes derivatives. As used herein the term “derivative” of a compound of the disclosure refers to a chemically modified compound wherein the chemical modification takes place either at a functional group or ring of the compound. Non-limiting examples of derivatives of compounds of the disclosure may include N-acetyl, N-methyl, or N-hydroxy groups at any of the available nitrogens in the compound.

A compound of the disclosure is a pharmaceutically functional derivative. A “pharmaceutically functional derivative” includes any pharmaceutically acceptable derivative of a compound of the disclosure, for example, an ester or an amide, which upon administration to a subject is capable of providing (directly or indirectly) a compound of the disclosure or an active metabolite or residue thereof. Such derivatives are recognizable to those skilled in the art, without undue experimentation (see for example Burger's Medicinal Chemistry and Drug Discovery, 5th Edition, Vol 1: Principles and Practice, which has illustrative pharmaceutically functional derivatives).

The term “substantially pure” as used herein means an object species is the predominant species present (i.e., on a molar basis it is more abundant than any other individual species in the composition), and preferably a substantially purified fraction is a composition wherein the object species comprises at least about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

The term “generating an image” as used herein refers to acquiring a detectable signal generated from a luciferase light source according to the present disclosure and determining the location of the source in a cell or an animal or human tissue. The intensity of the detectable signal may also be quantified.

The term “complemented RL activity” as used herein refers to the luciferase activity generated by the association of two polypeptides derived from a luciferase that, when in proximity to one another interact to provide a detectable luciferase-generated signal. The term may also apply to other species of luciferase including, but not limited to, a human codon-optimized Renilla luciferase.

The terms “polypeptide” and “protein” as used herein refer to a polymer of amino acids of three or more amino acids in a serial array, linked through peptide bonds. The term “polypeptide” includes proteins, protein fragments, protein analogues, oligopeptides and the like. The term “polypeptides” contemplates polypeptides as defined above that are encoded by nucleic acids, produced through recombinant technology, isolated from an appropriate source or are synthesized. The term “polypeptides” further contemplates polypeptides as defined above that include chemically modified amino acids or amino acids covalently or non-covalently linked to labeling ligands.

The term “fragment” as used herein to refer to a nucleic acid (e.g., cDNA) refers to an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase or any other polymerizing technique well known in the art, or expressed in a host cell by recombinant nucleic acid technology well known to one of skill in the art. The term “fragment” as used herein may also refer to an isolated portion of a polypeptide, wherein the portion of the polypeptide is cleaved from a naturally occurring polypeptide by proteolytic cleavage by at least one protease, or is a portion of the naturally occurring polypeptide synthesized by chemical methods well known to one of skill in the art.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein or polypeptide or a portion thereof.

The term “coding region” as used herein refers to a continuous linear arrangement of nucleotides that may be translated into a protein. A full-length coding region is translated into a full-length protein; that is, a complete protein as would be translated in its natural state absent of any post-translational modifications. A full-length coding region may also include any leader protein sequence or any other region of the protein that may be excised naturally from the translated protein.

The term “cancer” as used herein shall be given its ordinary meaning and is a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer.

The compounds described herein may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al., (eds.), Vogel's Encyclopedia of Practical Organic Chemistry 5th ed., Longman Scientific and Technical Ltd., Essex, 1991, pp. 809-816; and Heller (1990) Acc. Chem. Res. 23: 128.

Where a disclosed compound includes a conjugated ring system, resonance stabilization may permit a formal electronic charge to be distributed over the entire molecule. While a particular charge may be depicted as localized on a particular ring system, or a particular heteroatom, it is commonly understood that a comparable resonance structure can be drawn in which the charge may be formally localized on an alternative portion of the compound.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl”, either alone or within other terms such as “thioalkyl” and “arylalkyl”, means a monovalent, saturated hydrocarbon radical which may be a straight chain (i.e. linear) or a branched chain. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g., CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

In aspects of the disclosure, “substituted alkyl” includes an alkyl group substituted by, for example, one to five substituents, and preferably 1 to 3 substituents, such as alkyl, alkoxy, oxo, alkanoyl, aryl, aralkyl, aryloxy, alkanoyloxy, cycloalkyl, acyl, amino, hydroxyamino, alkylamino, arylamino, alkoxyamino, aralkylamino, cyano, halogen, hydroxyl, carboxyl, carbamyl, carboxylalkyl, keto, thioketo, thiol, alkylthiol, arylthio, aralkylthio, sulfonamide, thioalkoxy; and nitro.

The term “substituted aliphatic” as used herein refers to an alkyl or an alkane possessing less than 10 carbons. The term “substituted aliphatic” refers to an alkyl or an alkane possessing less than 10 carbons where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such groups include, but are not limited to, 1-chloroethyl and the like.

The term “lower-alkyl-substituted-amino” as used herein refers to any alkyl unit containing up to and including eight carbon atoms where one of the aliphatic hydrogen atoms is replaced by an amino group. Examples of such include, but are not limited to, ethylamino and the like.

The term “lower-alkyl-substituted-halogen” as used herein refers to any alkyl chain containing up to and including eight carbon atoms where one of the aliphatic hydrogen atoms is replaced by a halogen. Examples of such include, but are not limited to, chlorethyl and the like.

The term “acetylamino” shall mean any primary or secondary amino that is acetylated. Examples of such include, but are not limited to, acetamide and the like.

The term “alkenyl” as used herein refers to an unsaturated, acyclic branched or straight-chain hydrocarbon radical comprising at least one double bond. An alkenyl radical may contain from about 2 to 24 or 2 to 10 carbon atoms, in particular from about 3 to 8 carbon atoms and more particularly about 3 to 6 or 2 to 6 carbon atoms. Suitable alkenyl radicals include without limitation ethenyl, propenyl (e.g., prop-1-en-1-yl, prop-1-en-2-yl, prop-2-en-1-yl (allyl), and prop-2-en-2-yl), buten-1-yl, but-1-en-2-yl, 2-methyl-prop-1-en-1-yl, but-2-en-1-yl, but-2-en-2-yl, buta-1,3-dien-1-yl, beta-1,3-dien-2-3/1, hexen-1-yl, 3-hydroxyhexen-yl, hepten-1-yl, and octen-1-yl, and the like. An alkenyl radical may be optionally substituted similar to alkyl.

The term “substituted alkenyl” as used herein includes an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkynyl” as used herein refers to an unsaturated, branched or straight-chain hydrocarbon radical comprising one or more triple bonds. An alkynyl radical may contain about 1 to 20, 1 to 15, or 2 to 10 carbon atoms, particularly about 3 to 8 carbon atoms and more particularly about 3 to 6 carbon atoms. Suitable alkynyl radicals include without limitation ethynyl, such as prop-1-yn-1-yl and prop-2-yn-1-yl, butyryls such as but-1-yn-1-yl, but-1-yn-3-yl, and but-3-yn-1-yl, pentynyls such as perityn-1-yl, pentyn-2-yl, 4-methoxypentyn-2-yl, and 3-methylbutyn-1-yl, hexynyls such as hexyn-1-yl, hexyn-2-yl, hexyn-3-yl, and 3,3-dimethylbutyn-1-yl radicals and the like. In aspects of the disclosure, alkenyl groups include ethenyl (—CH═CH₂), n-propenyl (—CH₂CH═CH₂), iso-propenyl (—C(CH₃)═CH₂), and the like. An alkynyl may be optionally substituted similar to alkyl. The term “cycloalkynyl” refers to cyclic alkynyl groups.

The term “substituted alkynyl” as used herein includes an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalitoxy, aryl, nitro, and the like.

The term “alkylene” as used herein refers to a linear or branched radical having from about 1 to 10, 1 to 8, 1 to 6, or 2 to 6 carbon atoms and having attachment points for two or more covalent bonds. Examples of such radicals are methylene, ethylene, propylene, butylene, pentylene, hexylene, ethylidene, methylethylene, and isopropylidene. When an alkenylene radical is present as a substituent on another radical it is typically considered to be a single substituent rather than a radical formed by two substituents.

The term “alkenylene” as used herein refers to a linear or branched radical having from about 2 to 10, 2 to 8 or 2 to 6 carbon atoms, at least one double bond, and having attachment points for two or more covalent bonds. Examples of alkenylene radicals include 1,1-vinylidene (—CH₂═C—), 1,2-vinylidene (—CH═CH—), and 1,4-butadienyl (—CH═CH—CH═CH—).

The term “halo” as used herein refers to a halogen such as fluorine, chlorine, bromine or iodine atoms.

The term “hydroxyl” or “hydroxy” as used herein refers to an —OH group.

The term “cyano” as used herein refers to a carbon radical having three of four covalent bonds shared by a nitrogen atom, in particular —CN. A cyano group may be substituted with substituents described herein.

The term “alkoxy” refers to a linear or branched oxy-containing radical having an alkyl portion of one to about ten carbon atoms, such as a methoxy radical, which may be substituted.

In aspects of the disclosure an alkoxy radical may comprise about 1-10, 1-8, 1-6 or 1-3 carbon atoms. In embodiments of the disclosure, an alkoxy radical comprises about 1-6 carbon atoms and includes a C₁-C₆ alkyl-O-radical wherein C₁-C₆ alkyl has the meaning set out herein. Examples a alkoxy radicals include without limitation methoxy, ethoxy, propoxy, butoxy, isopropoxy and tert-butoxy alkyls. An “alkoxy” radical may, optionally be substituted with one or more substitutents disclosed herein including alkyl atoms to provide “alkylalkoxy” radicals; halo atoms, such as fluoro, chloro or bromo, to provide “haloalkoxy” radicals (e.g., fluoromethoxy, chloromethoxy, trifluoromethoxy, difluoromethoxy, trifluoroethoxy, fluoroethoxy, tetrafluoroethoxy, pentafluoroethoxy, and fluoropropox) and “haloalkoxyalkyl” radicals (e.g., fluoromethoxymethyl, chloromethoxyethyl, trifluorornethoxymethyl, difluoromethoxyethyl, and trifluorocthoxymethyl).

The term “alkenyloxy” as used herein refers to linear or branched oxy-containing radicals having an alkenyl portion of about 2 to 10 carbon atoms, such as an ethenyloxy or propenyloxy radical. An alkenyloxy radical may be a “lower alkenyloxy” radical having about 2 to 6 carbon atoms. Examples of alkenyloxy radicals include without limitation ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g., trifluoroethenyloxy, fluoroethenyloxy, difluoroethenyloxy, and fluoropropenyloxy).

The term “carbocylic” as used herein includes radicals derived from a saturated or unsaturated, substituted or unsubstituted 5 to 14 member organic nucleus whose ring forming atoms (other than hydrogen) are solely carbon. Examples of carbocyclic radicals are cycloalkyl, cycloalkenyl, aryl, in particular phenyl, naphthyl, norbornanyl, bicycloheptadienyl, toluoyl, xylenyl, indenyl, stilbenzyl, terphenylyl, diphenylethylenyl, phenylcyclohexyl, acenapththylenyi, anthracenyl, biphenyl, bibenzylyl, and related bibenzylyl homologs, octahydronaphthyl, tetrahydronaphthyl, octahydroquinolinyl, dimethoxytetrahydronaphthyl and the like.

The term “cycloalkyl” as used herein refers to radicals having from about 3 to 15, 3 to 10, 3 to 8, or 3 to 6 carbon atoms and containing one, two, three, or four rings wherein such rings may be attached in a pendant manner or may be fused. In aspects of the disclosure, “cycloalkyl” refers to an optionally substituted, saturated hydrocarbon ring system containing 1 to 2 rings and 3 to 7 carbons per ring which may be further fused with an unsaturated C3-C7 carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. Tin certain aspects of the disclosure the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted with groups as disclosed herein.

The term “substituted cycloalkyl” as used herein includes cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

The term “cycloaliphatic” refers to a cycloalkane possessing less than 8 carbons or a fused ring system consisting of no more than three fused cycloaliphatic rings. Examples of such groups include, but are not limited to, decalin and the like.

The term “substituted cycloaliphatic” as used herein refers to a cycloalkane possessing less than 8 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to, 1-chlorodecalyl and the like.

The term “cycloalkenyl” as used herein refers to radicals comprising about 4 to 16, 2 to 15, 2 to 10, 2 to 8, 4 to 10, 3 to 8, 3 to 7, 3 to 6, or 4 to 6 carbon atoms, one or more carbon-carbon double bonds, and one, two, three, or four rings wherein such rings may be attached in a pendant manner or may be fused. In certain aspects of the disclosure the cycloalkenyl radicals are “lower cycloalkenyl” radicals having three to seven carbon atoms. Examples of cycloalkenyl radicals include without limitation cyclobutenyl, cyclopentenyl, cyclohexenyl and cycloheptenyl. A cycloalkenyl radical may be optionally substituted with groups as disclosed herein, in particular 1, 2, or 3 substituents which may be the same or different.

The term “cycloalkoxy” as used herein refers to cycloalkyl radicals (in particular, cycloalkyl radicals having 3 to 15, 3 to 8 or 3 to 6 carbon atoms) attached to an oxy radical. Examples of cycloalkoxy radicals include cyclohexoxy and cyclopentoxy. A cycloalkoxy radical may be optionally substituted with groups as disclosed herein.

The term “aryl”, alone or in combination, as used herein refers to a carbocyclic aromatic system containing one, two or three rings wherein such rings may be attached together in a pendant manner or may be fused, in aspects of the disclosure an aryl radical comprises 4 to 24 carbon atoms, in particular 4 to 10, 4 to 8, or 4 to 6 carbon atoms. Illustrative “aryl” radicals includes without limitation aromatic radicals such as phenyl, benzyl, naphthyl, indenyl, benzocyclooctenyl, benzocycloheptenyl, pentalenyl, azulenyl, tetrahydronaphthyl, indanyl, biphenyl, acephthylenyl, fluorenyl, phenalenyl, phenanthrenyl, and anthracenyl, preferably phenyl.

An aryl radical may be optionally substituted with groups as disclosed herein, in particular hydroxyl, alkyl, carbonyl, carboxyl, thiol, amino, and/or halo, in particular a substituted aryl includes without limitation arylamine and arylalkylamine.

The term “substituted aryl” as used herein includes an aromatic ring, or fused aromatic ring system consisting of no more than three fused rings at least one of which is aromatic, and where at least one of the hydrogen atoms on a ring carbon has been replaced by a halogen, an amino, a hydroxy, a nitro, a thio, an alkyl, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl, chlorphenyl and the like.

An aryl radical may be optionally substituted with one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, acylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfenic acid, alkysulfonyl, sulfonamido, aryloxy and the like. A substituent may be further substituted by hydroxy, halo, alkyl, alkoxy, alkenyl, alkynyl, aryl or aralkyl. In aspects of the disclosure an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenzyl, and amino benzyl.

The term “aryloxy” as used herein refers to aryl radicals, as defined above, attached to an oxygen atom. Exemplary aryloxy groups include napthyloxy, quinolyloxy, isoquiriolizinyloxy, and the like.

The term “arylalkoxy” as used herein refers to an aryl group attached to an alkoxy group. Representative examples of arylalkoxy groups include, but are not limited to, 2-phenylethoxy, 3-naphth-2-ylpropoxy, and 5-phenylpentyloxy.

The term “aroyl” as used herein refers to aryl radicals, as defined above, attached to a carbonyl radical as defined herein, including without limitation benzoyl and toluoyl. An aroyl radical may be optionally substituted with groups as disclosed herein.

The term “heteroaryl” as used herein refers to fully unsaturated heteroatom-containing ring-shaped aromatic radicals having at least one heteroatom selected from carbon, nitrogen, sulfur and oxygen. A heteroaryl radical may contain one, two or three rings and the rings may be attached in a pendant manner or may be fused. In aspects of the disclosure the term refers to fully unsaturated hetoreatom-containing ring-shaped aromatic radicals having from 3 to 15, 3 to 10, 3 to 8, 5 to 15, 5 to 10, or 5 to 8 ring members selected from carbon, nitrogen, sulfur and oxygen, wherein at least one ring atom is a heteroatom. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, quinazolinyl, pteridinyl, quinolizidinyl, phthalazinyl, naphthyridinyl, quinoxalinyl, cinnolinyl, phenanthridinyl, acridinyl, phenanthrolinyl, phenazinyl, carbazolyl; purinyl, benzimidazolyl, quinolinyl, isoquinolinyl, beazotriazolyl, tetrazolopyridazinyl and the like; an unsaturated 3 to 6-membered heteromonocyclic group containing an oxygen atom, in particular, 2-furyl, pyranyl, and the like; an unsaturated 5 to 6-membered heteromonocyclic group containing a sulfur atom, in particular, thienyl, 2-thienyl, 3-thienyl, and the like; unsaturated 5 to 6-membered heteromonocyclic group containing 1 to 2 oxygen atoms and 1 to 3 nitrogen atoms, in particular, furazanyl, benzofurazanyl, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, isothiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuranyl, benzothiophenyl, phthalazinyl, chromenyl, xanthenyl, and the like. A heteroaryl radical may be optionally substituted with groups as disclosed herein, for example with an alkyl, amino, halogen, etc., in particular a heteroarylamine. The term may refer to an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, pyridinyl, pyrimidinyl, pyrazinyl, pyridazinyl, triazolyl, tetrazolyl and the like. A heteroaryl radical may be optionally substituted with groups disclosed herein, for example with an alkyl, amino, halogen, etc., in particular a substituted heteroaryl radical is a heteroarylamine.

The term “heterocyclic” as used herein refers to saturated and partially saturated heteroatom containing ring-shaped radicals having at least one heteroatom selected from carbon, nitrogen, sulfur and oxygen. A heterocylic radical may contain one, two or three rings wherein such rings may be attached in a pendant manner or may be fused. In an aspect, the term refers to a saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. Exemplary saturated heterocyclic radicals include without limitation a saturated 3 to 6-membered heteromonocylic group containing 1 to 4 nitrogen atoms (e.g., pyrrolidinyl, imidazolidinyl, and piperazinyl); a saturated 3 to 6-membered heteromonocyclic group containing 1 to 2 oxygen atoms and 1 to 3 nitrogen atoms (e.g., morpholinyl; sydnonyl); and, a saturated 3 to 6-membered heteromonocyclic group containing 1 to 2 sulfur atoms and 1 to 3 nitrogen atoms (e.g., thiazolidinyl) and the like. Examples of partially saturated heterocyclyl radicals include without limitation dihydrothiophene, dihydropyranyl, dihydrofuranyl and dihydrothiazolyl. Illustrative heterocyclic radicals include without limitation aziridinyl, azetidinyl, 2-pyrrolinyl, 3-pyrrolinyl, pyrrolidinyl, azepinyl, 1,3-dioxolanyl, 211-pyranyl, 4H-pyranyl, piperidinyl, 1,4-dioxanyl, morpholinyl, pyrazolinyl, thiomorpholinyl, 1,2,3,6-tetrahydropyridinyl, oxiranyl, oxetanyl, tetrahydrofuranyl, tetrahydropyranyl, tetrahydropyridinyl, tetrahydrothiopyranyl, thioxanyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, quinuelidinyl, quinolizinyl, and the like.

The term “heterocyclic” as used herein refers to a cycloalkane and/or an aryl ring system, possessing less than 8 carbons, or a fused ring system consisting of no more than three fused rings, where at least one of the ring carbon atoms is replaced by oxygen, nitrogen or sulfur. Examples of such groups include, but are not limited to, morpholino and the like.

The term “substituted heterocyclic” as used herein refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amid; a lower aliphatic, a substituted lower aliphatic, or a ring (aryl, substituted aryl, cycloaliphatic, or substituted cycloaliphatic). Examples of such groups include, but are not limited to 2-chloropyranyl. The foregoing heteroaryl and heterocyclic groups may be C-attached or N-attached (where such is possible).

The term “sulfonyl” as used herein used alone or linked to other terms such as alkylsulfonyl or arylsulfonyl, refers to the divalent radicals —SO₂⁻. In aspects of the disclosure, the sulfonyl group may be attached to a substituted or unsubstituted hydroxyl, alkyl group, ether group, alkenyl group, alkynyl group, aryl group, cycloalkyl group, cycloalkenyl group, cycloalkynyl group, heterocyclic group, carbohydrate, peptide, or peptide derivative.

The term “sulfinyl” as used herein, used alone or linked to other terms such as alkylsulfinyl (i.e., —S(O)-alkyl) or arylsulfinyl, refers to the divalent radicals —S(O)—.

The term “sulfoxide” refers to the radical —S═O.

The term “amino” as used herein, alone or in combination, refers to a radical where a nitrogen atom (N) is bonded to three substituents being any combination of hydrogen, hydroxyl, alkyl, cycloalkyl, alkenyl, alkynyl, aryl, silyl, heterocyclic, or heteroaryl which may or may not be substituted. Generally an “amino group” has the general chemical formula —NR₂₀R₂₁ where R₂₀ and R₂₁ can be any combination of hydrogen, hydroxyl, alkyl, cycloalkyl, alkoxy, alkenyl, alkynyl, aryl, carbonyl carboxyl, amino, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidinyl, hydrazinyl, benzylamino, diphenylmethylamino, tritylamino, trimethylsilylamino, and dimethyl-tert.-butylsilyiamino, which may or may not be substituted.

Term “thiol” as used herein means —SH. A thiol may be substituted with a substituent disclosed herein, in particular alkyl (thioalkyl), aryl (thioaiyl), alkoxy (thioalkoxy) or carboxyl.

The term “sulfenyl” as used herein used alone or linked to other terms such as alkylsulfenyl, refers to the radical —SR₂₂ wherein R₂₂ is not hydrogen. In aspects of the disclosure R₂₂ is substituted or unsubstituted alkyl, cycloalkyl, alkenyl, alkynyl, aryl, silyl, silylalkyl, heterocyclic, heteroaryl, carbonyl, carbamoyl, alkoxy, or carboxyl.

The term “thioalkyl” as used herein, alone or in combination, refers to a chemical functional group where a sulfur atom (5) is bonded to an alkyl, which may be substituted. Examples of thioalkyl groups are thiomethyl, thioethyl, and thiopropyl. A thioalkyl may be substituted with a substituted or unsubstituted carboxyl, aryl, heterocylic, carbonyl, or heterocyclic.

The term “thioaryl” as used herein, alone or in combination, refers to a chemical functional group where a sulfur atom (S) is bonded to an aryl group with the general chemical formula —SR₂₃ where R₂₃ is aryl which may be substituted. Illustrative examples of thioaryl groups and substituted thioaryl groups are thiophenyl, chlorothiophenol, para-chlorothiophenol, thiobenzyl, 4-methoxy-thiophenyl, 4-nitro-thiophenyl, and para-nitrothiobenzyl.

The term “thioalkoxy” as used herein, alone or in combination, refers to a chemical functional group where a sulfur atom (S) is bonded to an alkoxy group with the general chemical formula —SR₂₄ where R₂₄ is an alkoxy group which may be substituted. A “thioalkoxy group” may have 1-6 carbon atoms i.e. a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

A thiol may be substituted with a substituted or unsubstituted heteroaryl or heterocyclic, in particular a substituted or unsubstituted saturated 3 to 6-membered heteromonocylic group containing 1 to 4 nitrogen atoms (e.g., pyrrolidinyl, imidazolidinyl, piperidinyl, and piperazinyl) or a saturated 3 to 6-membered heteromonocyclic group containing 1 to 2 oxygen atoms and 1 to 3 nitrogen atoms (e.g., morpholinyl; sydrionyl), especially a substituted morpholinyl or piperidinyl.

The term “carbonyl” as used herein refers to a carbon radical having two of the four covalent bonds shared with an oxygen atom.

The term “carboxyl” as used herein, alone or in combination, refers to —C(O)OR₂₅— or —C(—O)OR₂₅ wherein R₂₅ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl groups. In particular aspects of the disclosure, —C(O)OR₂₅ provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert.butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.butylcarborlyl, 4-nitrobenzyloxycarbonyl, di phenyl methoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g., methyl), alkoxy (e.g., methoxy), and/or halo (e.g., chlorine). Examples of silicon substituents include trimethylsilyi and dimethyltert.butylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl.

The term “carbamoyl” as used herein, alone or in combination, refers to amino, monoalkylamino, dialkylamino, monocycloalkylamino, alkyleycloalkylamino, and dicycloalkylaxaino radicals, attached to one of two unshared bonds in a carbonyl group.

The term “carboxamide” as used herein refers to the group —CONH—.

The term “nitro” as used herein means —NO₂—.

The term “acyl” as used herein, alone or in combination, means a carbonyl or thiocarbonyl group bonded to a radical selected from, for example, optionally substituted, hydrido, alkyl (e.g., haloalkyl), alkenyl, alkynyl, alkoxy (“acyloxy” including acetyloxy, butyryloxy, iso-valeryloxy, phenylacetyloxy, berizoyloxy, p-methoxybenzoyloxy, and substituted acyloxy such as alkoxyalkyl and haloalkoxy), aryl, halo, heterocyclyl, heteroaryl, sulfonyl (e.g., allylsulfinylalkyl), sulfonyl (e.g., alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like. The term “acyl” as used herein refers to a group —C(O)R₂₆, where R₂₆ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, beozylcarbonyl and the like.

The term “phosphonate” refers to a C—PO(OH)₂ or C—PO(OR₂₇)₂ group wherein R₂₇ is alkyl or aryl which may be substituted.

The terms for radicals including “alkyl”, “alkoxy”, “alkenyl”, “alkynyl”, “hydroxyl” etc. as used herein refer to both unsubstituted and substituted radicals. The term “substituted,” as used herein, means that any one or more moiety on a designated atom (e.g., hydrogen) is replaced with a selection from a group disclosed herein, provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

A functional group or ring of a compound of the disclosure may be modified with, or a radical in a compound of the disclosure may be substituted with one or more groups or substituents apparent to a person skilled in the art including without limitation alkyl, alkoxy, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, alkylsulfonyl, sulfinyl, sulfonyl, sulfenyl, alkylsulfinyl, aralkyl, heteroaralkyl, cycloalkyl, cyclo alkenyl, cycloalkoxy, cycloalkenyloxy, amino, oxy, halo, azido, thio, .═O, .═S, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfony, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. These groups or substitutents may themselves be substituted.

The term “dosage form” as used herein refers to a composition or device comprising a compound of the disclosure and optionally pharmaceutically acceptable carrier(s), excipient(s), or vehicles. A dosage form may be an immediate release dosage form or a sustained release, dosage form. An “immediate release dosage form” refers to a dosage form which does not include a component for sustained release i.e., a component for slowing disintegration or dissolution of an active compound. These dosage forms generally rely on the composition of the drug matrix to effect the rapid release of the active ingredient agent. By “sustained release dosage form” is meant a dosage form that releases active compound for many hours. In an aspect, a sustained dosage form includes a component for slowing disintegration or dissolution of the active compound. A dosage form may be a sustained release formulation, engineered with or without an initial delay period. Sustained release dosage forms may continuously release drug for sustained periods of at least about 4 h or more, about 6 h or more, about 8 h or more, about 12 h or more, about 15 h or more, or about 20 h to 24 h. A sustained release dosage form can be formulated into a variety of forms, including tablets, lozenges, gelcaps, buccal patches, suspensions, solutions, gels, etc. In aspects of the disclosure the sustained release form results in administration of a minimum number of daily doses.

### DESCRIPTION

The present disclosure encompasses embodiments of methods for identifying novel inhibitors of Hsp90 chaperone protein folding. The inhibitors identified by the methods of the disclosure disrupt the binding of p23 to either Hsp90α or Hsp90β.

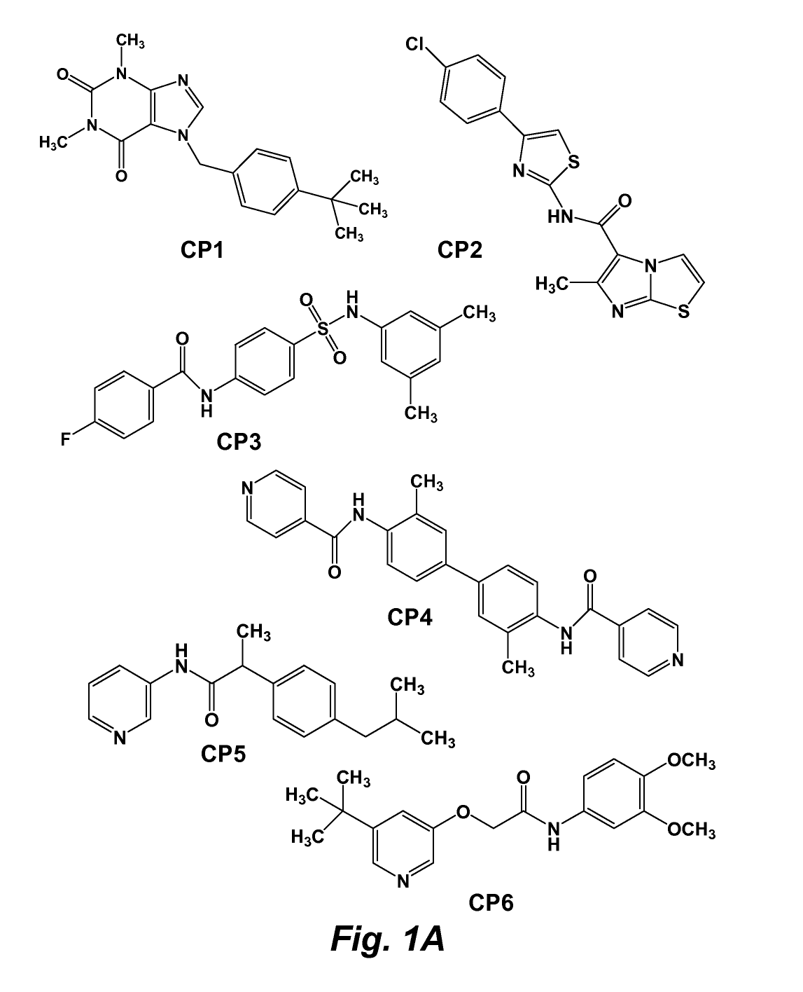

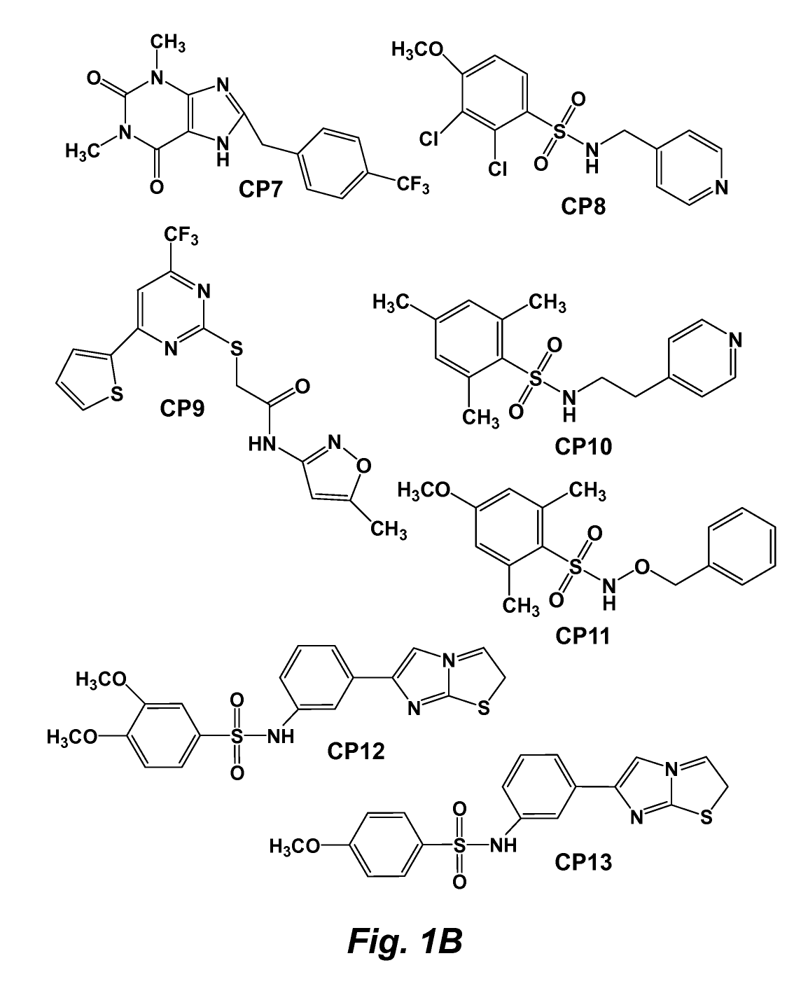

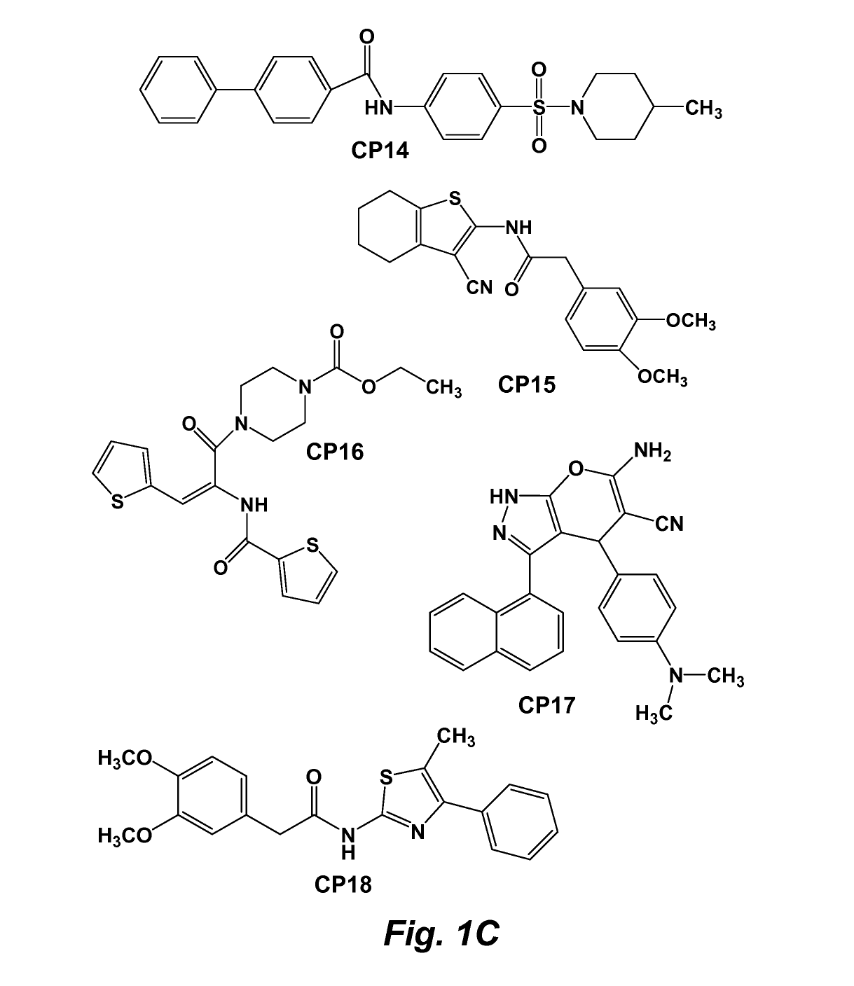

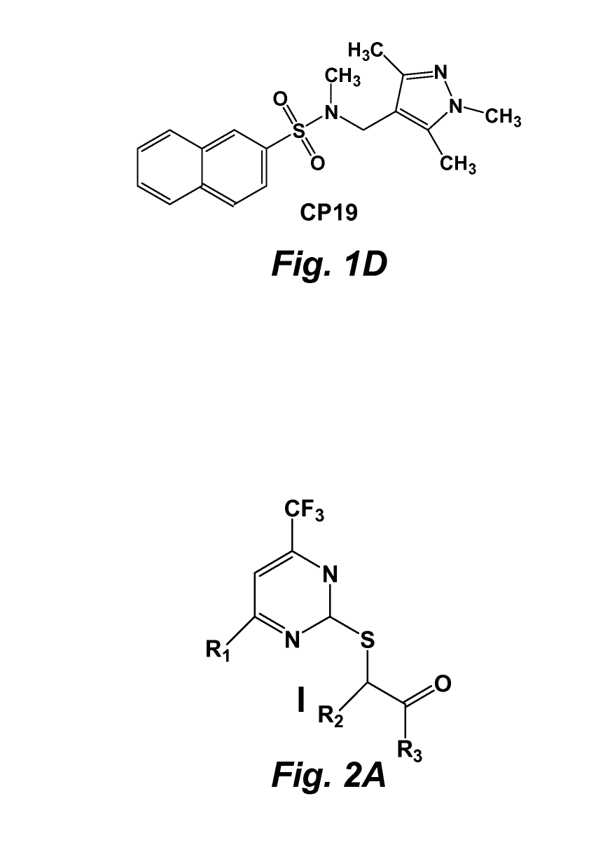

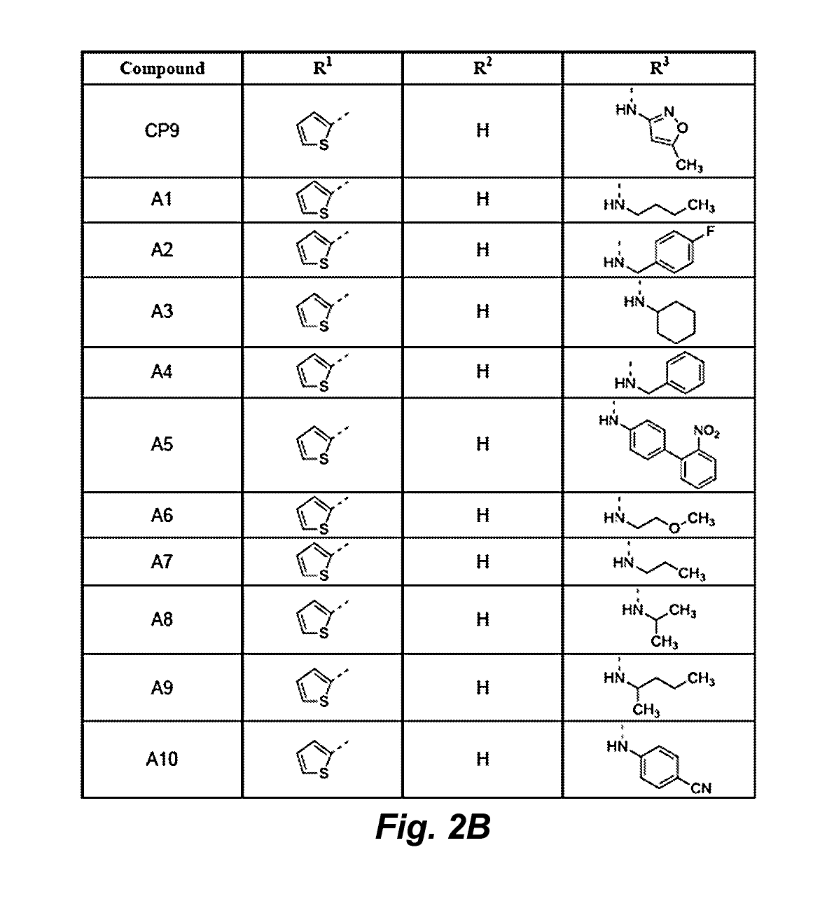

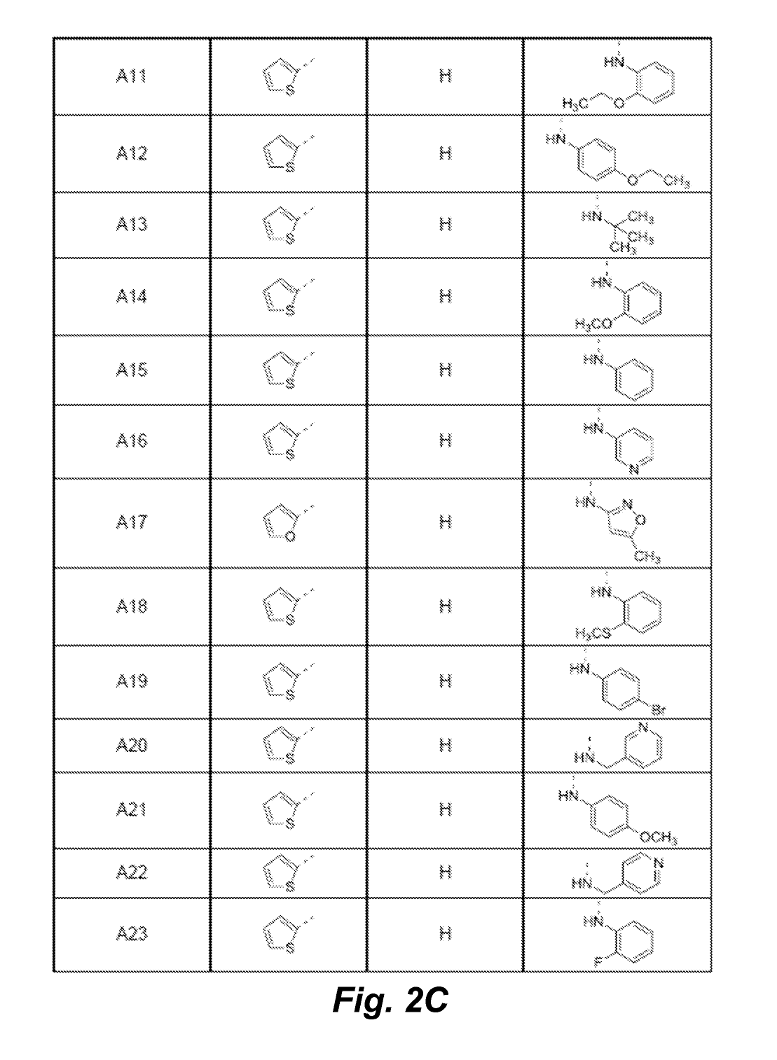

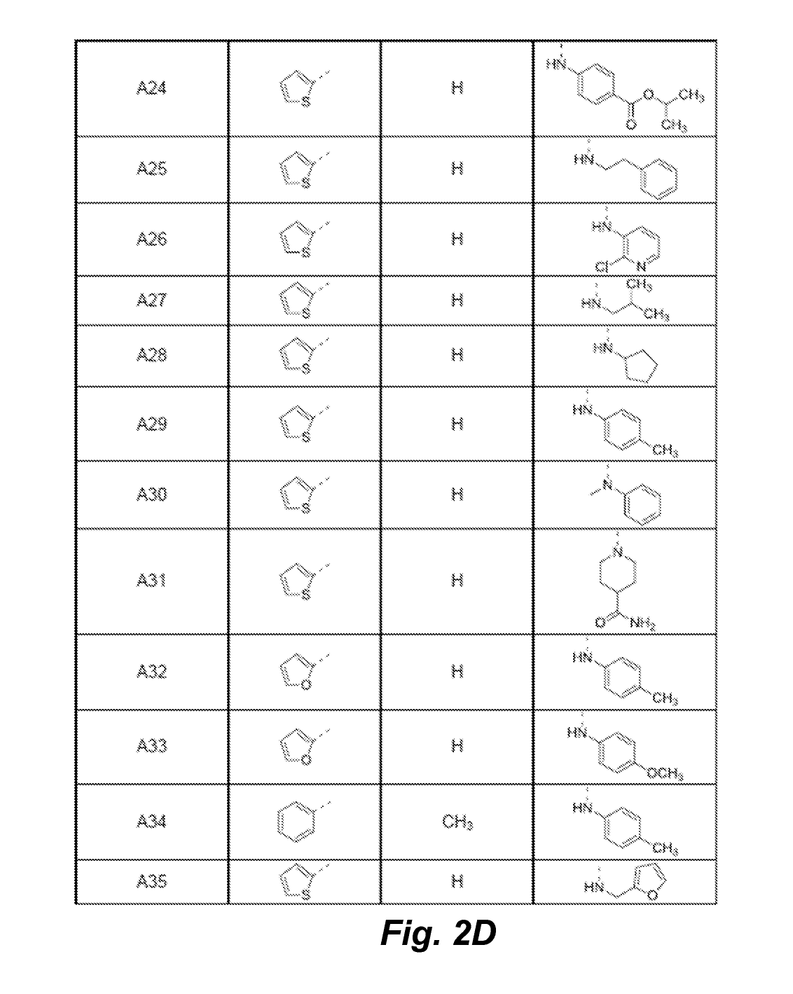

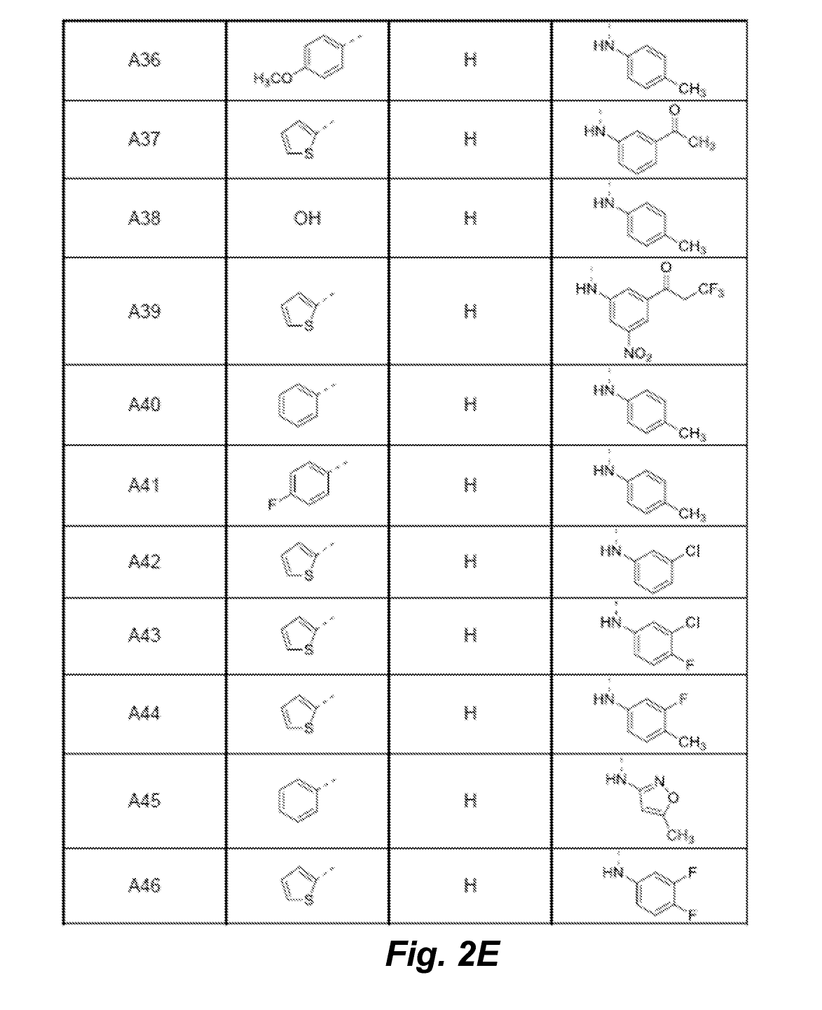

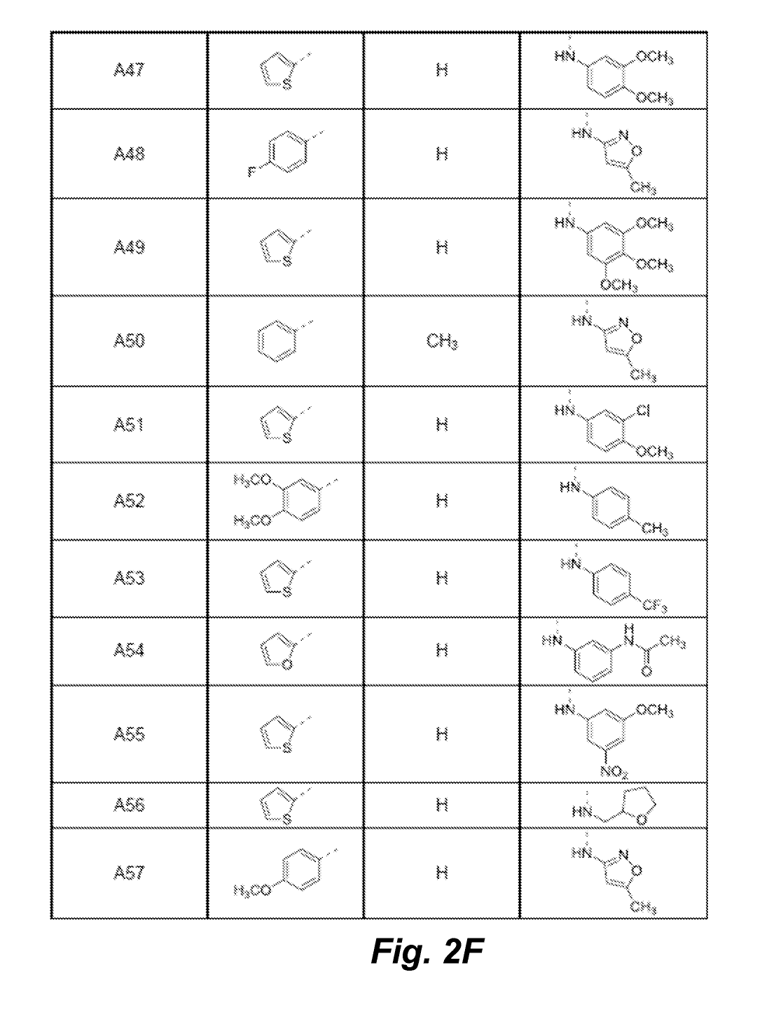

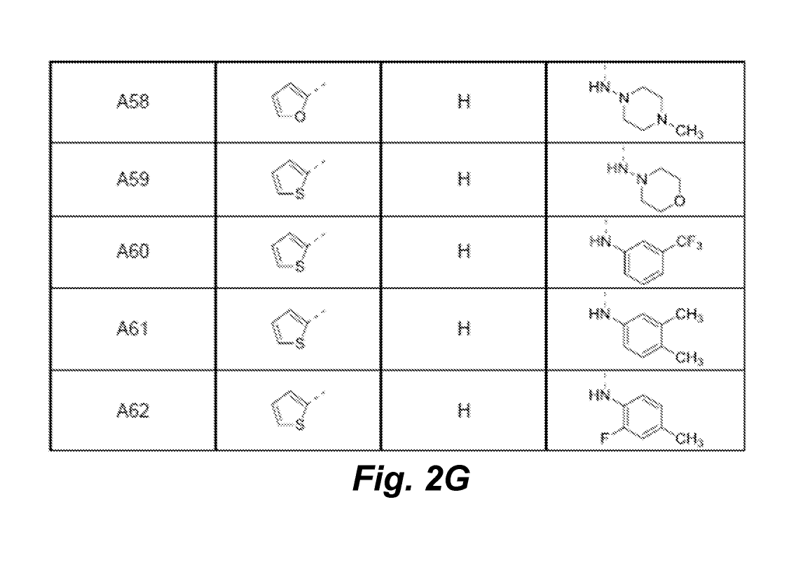

In particular, the disclosure encompasses embodiments of therapeutic compositions that comprise at least one inhibitor of an Hsp90 chaperone activity, the inhibitor being any of the compounds designated as CP1-CP19 as shown in FIGS. 1A-1D or a compound having the formula I:

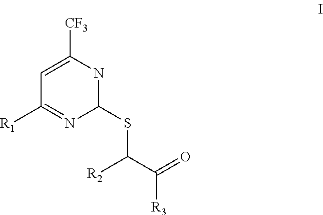

wherein R₁ can be a thiophene, a furan, a substituted or unsubstituted phenyl, or —OH; R₂ can be H or an alkyl; and R₃ can be phenylmethylamine, 4-amidopyridyl, or —NHR₄, wherein R₄ can be a substituted isoxazole, a substituted or unsubstituted alkyl, a substituted or unsubstituted branched chain alkyl, a substituted or unsubstituted —(CH)ₙPh, a substituted or unsubstituted 5 or 6-membered aryl, a substituted or unsubstituted 5 or 6-membered heteroaryl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine, and where the therapeutic composition is formulated to have a dose of the inhibitor effective in reducing the viability of a cancer cell when delivered to an animal or human. It is further contemplated that the inhibitor may be, but is not limited to, any of the compounds A1-A62 as shown in FIGS. 2A-2G. Especially advantageous compounds of the disclosure are 2-(trifluoromethyl)pyrimidin-2-yl)thio)acetamide derivatives including, but not limited to, compound CP9 as shown in FIG. 1B.

It is further contemplated to be in the scope of the disclosure for the inhibitor compounds to be a salt or other derivative that retains the ability of the compound to inhibit Hsp90 chaperone activity when administered to a subject animal or human. The compounds of the disclosure are especially advantageous for the selective reduction in the viability and/or the proliferative capacity of a cancer cell while having less or no effect on non-cancerous cells. Accordingly, the therapeutic compositions of the disclosure are formulated for administering to a subject in need of therapeutic treatment for a cancer.

The disclosure further provides a high-throughput screening (HTS) method for the identification of compounds that selectively inhibit the proliferation of cancer cells and also have in vivo activity in recipient animals. The HTS methods of the disclosure allow for the initial screening of Hsp90 chaperone inhibition activity by cell culture-based steps followed by administering of effective inhibitors to a recipient mouse having a xenograft tumor. The localization and ability of the compound to inhibit tumor growth may then be monitored by imaging procedures herein disclosed.

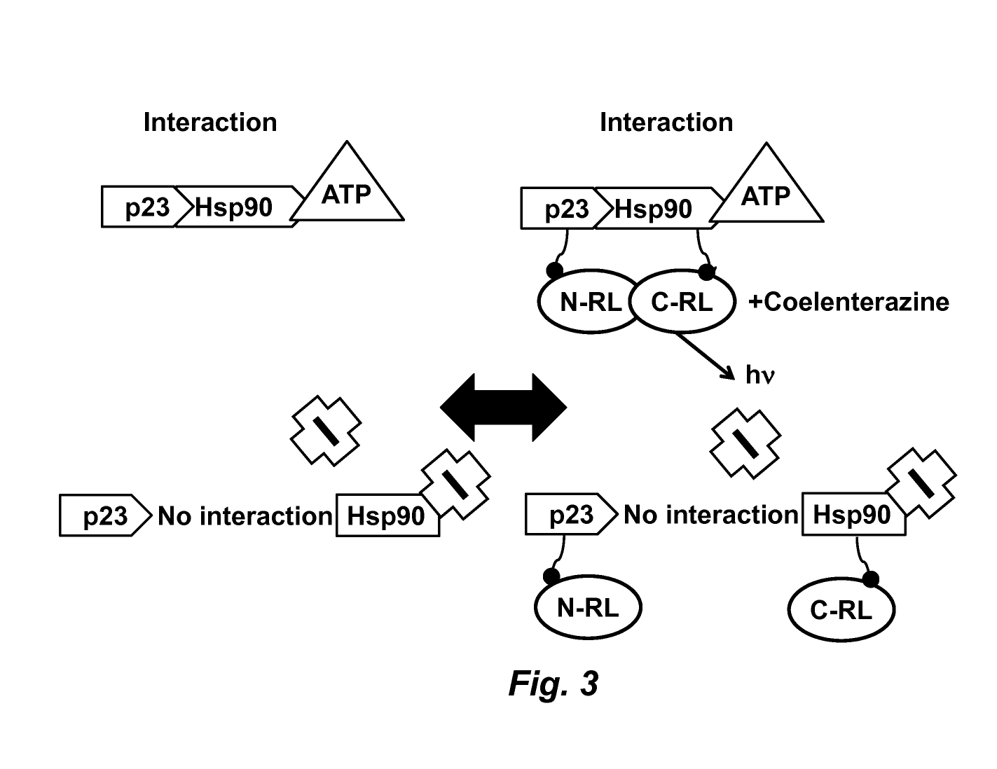

Initially, desirable compounds may be identified by a cell-based system that is readily adaptable as a high-throughput screening method. In embodiments of this system, a split luciferase assay system, as shown in FIG. 3, comprises a p23 polypeptide modified by the attachment of an N-terminal fragment of a Renilla luciferase thereto, and an isoform of Hsp90 polypeptide modified by the attachment of a C-terminus fragment of the luciferase thereto. When expressed in a target cell and in the presence of ATP, the modified p23 and the modified Hsp90 isoform complex and allow the two fragments of the split luciferase to associate and complement. When coelentarazine is added to the cells, the two complemented fragments cooperate to generate a detectable signal. Addition of a test or candidate inhibitor of the Hsp90/p23 complex formation will, if inhibitory, reduce the intensity of the emitted signal. The assay system may further comprise expressing in the target cells a bioluminescent reporter such as, but not limited to, FL-eGFP. In the presence of luciferin, a detectable signal is emitted thereby providing a determination of the number of target cells and whether they have proliferated. This signal can allow the operator to normalize the detected signal emitted by the split Renilla luciferase reporter, allowing comparisons quantitative comparisons between candidate inhibitors as to their efficacy to inhibit the Hsp90/p23 association, relative to carrier control treated cells.

The assays of the disclosure are also advantageous in rapidly determining whether a candidate Hsp90/p23 inhibitor can also function as an inhibitor of tumor formation. For this purpose, the system is adapted by obtaining a genetically-modified tumor cell comprising at least one recombinant nucleic acid expressing an N-terminal fragment of a Renilla luciferase attached to a p23 polypeptide and a C-terminus fragment of the luciferase attached to an isoform of Hsp90 polypeptide. Additionally, the genetically modified cells may be stably transfected with a nucleic acid expressing a bioluminescent reporter such as, but not limited to, FL-eGFP.

The cells may be delivered to a recipient mammal such as a mouse or rat to provide a xenograft. Growth of the cells will, therefore, form a localized tumor in the recipient subject, the developmental progress of which may be monitored by imaging methods following administration to the animal of a D-luciferin and imaging monitoring of the emitted bioluminescence by a cooled CCD camera. The effect of the administration of a candidate compound suspected of inhibiting the complexing of Hsp90 and p23 and thereby reducing the proliferation of the tumor-forming cells may be monitored by periodic imaging of a signal emitted by the tumor mass upon delivery of coelentarazine to the subject animal.

For example, to provide for the development of novel Hsp90 inhibitors, multimodality molecular imaging was combined with a HTS method to screen a randomized chemical library, thereby identifying a novel class of 2-((6-(trifluoromethyl)pyrimidin-2-yl)thio)acetamide-based Hsp90 inhibitors through molecular imaging of Hsp90(α/β)/p23 interactions.

Up-regulation of the Hsp90 chaperone protein folding machinery is crucial for cancer progression. The two Hsp90 isoforms (α/β) play different roles in response to chemotherapy. To identify isoform-selective Hsp90(α/β)/co-chaperone p23 interactions inhibitors, a dual-luciferase [Renilla (RL) and Firefly (FL)] reporter system was developed for high-throughput screening (HTS) and efficacy monitoring of these inhibitors in cell culture and mice. HTS of a 30,176 component small molecule chemical library in cell culture identified a new N-(5-methylisoxazol-3-yl)-2-(4-(thiophen-2-yl)-6-(trifluoromethyl)pyrimidin-2-ylthio)acetamide [CP9] that binds to Hsp90(α/β) and displays characteristics of Hsp90 inhibitors, i.e., degradation of Hsp90 client proteins and inhibition of cell proliferation, glucose metabolism and thymidine kinase activities in multiple cancer cell lines. The efficacy of CP9 in disruption of Hsp90(α/β)/p23 interactions and cell proliferation in tumor xenografts was evaluated by non-invasive, repetitive RL and FL imaging, respectively. At 38 h post treatment (80 mg/kg×3, i.p.), CP9 led to selective disruption of Hsp90α/p23, compared to that of Hsp90β/23 interactions. Small animal positron emission tomography/computed tomography [PET/CT] with the same cohort of mice showed that CP9 treatment (43 h) led to a 40% decrease ¹⁸F-Fluorodeoxyglucose [¹⁸F-FDG] uptake in tumors, relative to carrier control treated mice. However, CP9 did not significant degrade Hsp90 client proteins in tumors.

Structural activity relationships (SAR) were also determined for 62 analogues of CP9 and identified A17 as the lead compound that outperformed CP9 in inhibition of Hsp90(α/β)/p23 interactions in cell culture, thereby demonstrating the power of coupling of HTS with multimodality molecular imaging for the rapid discovery of novel Hsp90 inhibitors.

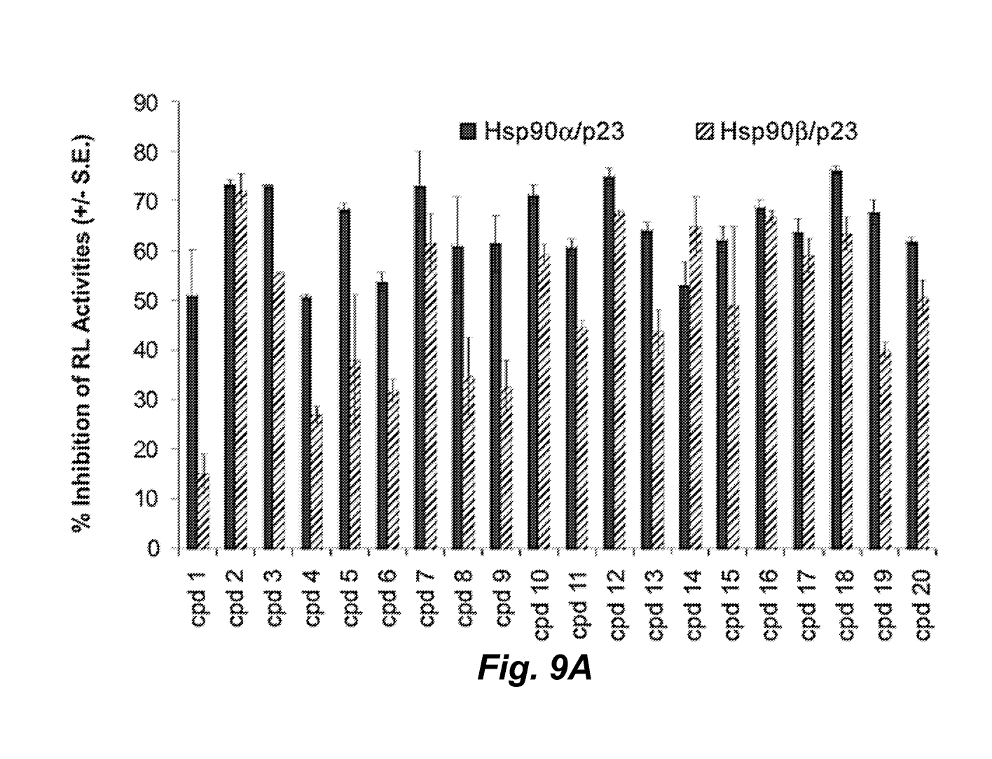

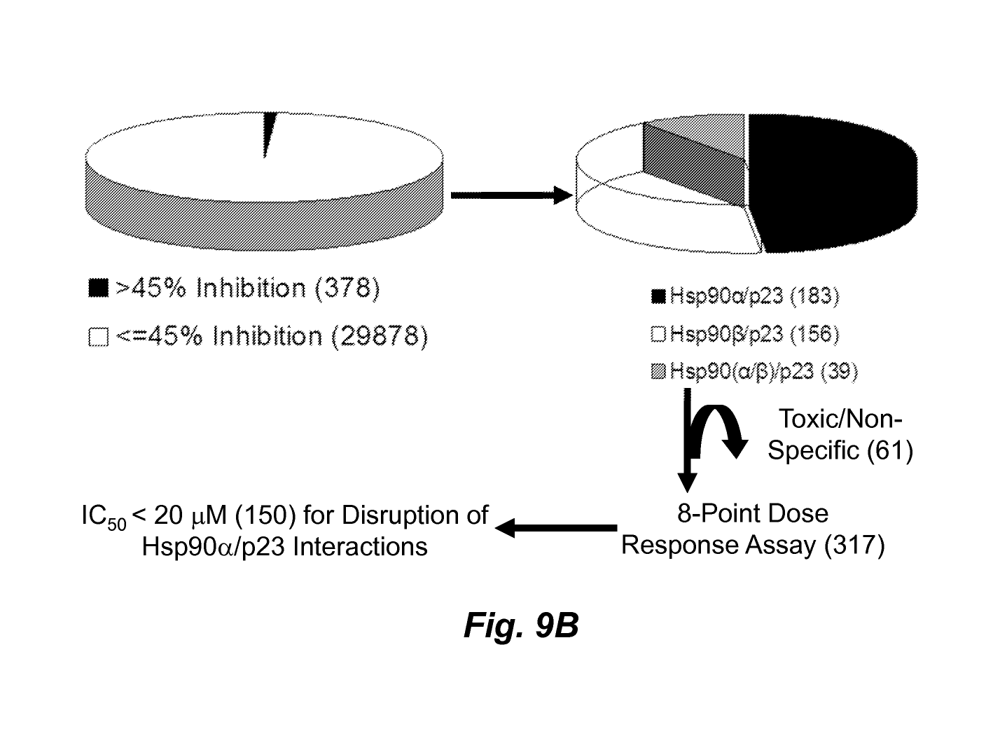

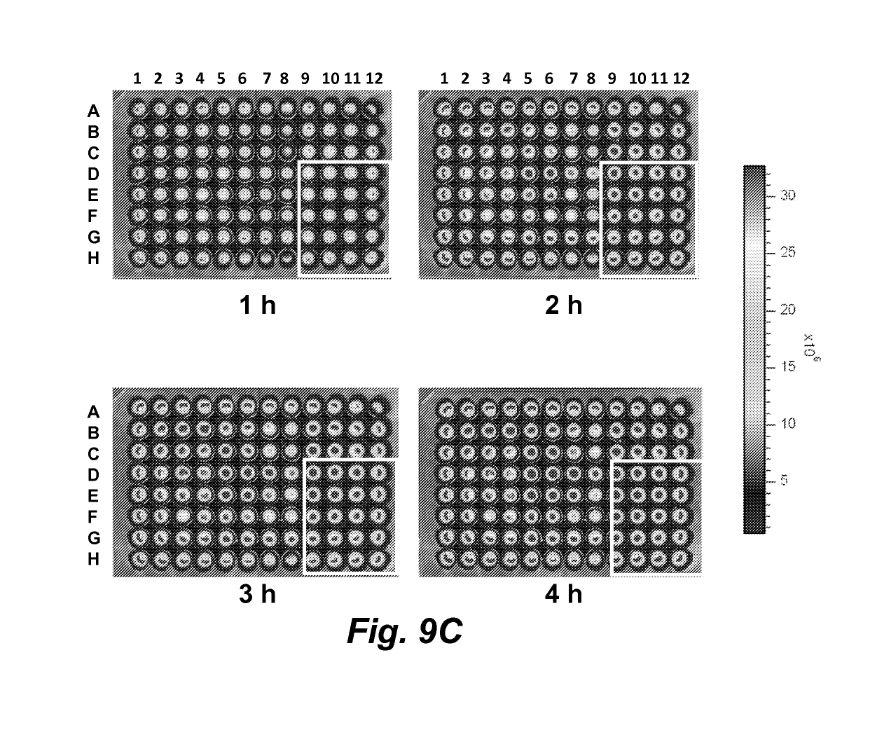

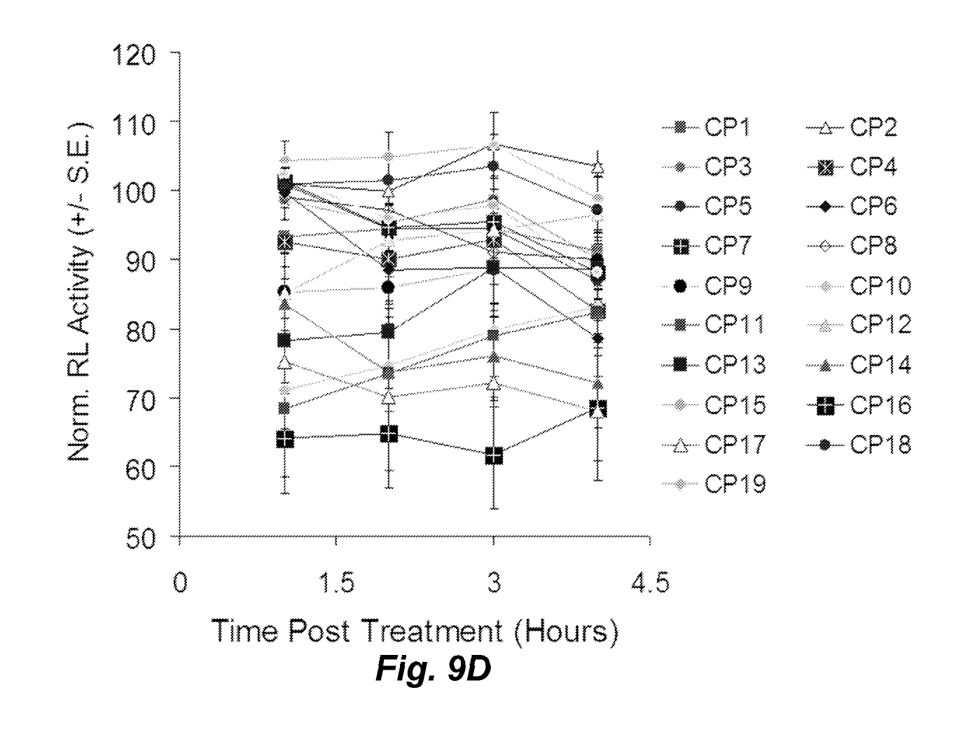

An initial screening was performed using the Library of Pharmacologically Active Compounds (LOPAC) (1280 compounds) (described in Inglese et al., (2006) Proc. Nat. Acad. Sc. U.S.A. 103: 11473-11478). 293T human embryonic kidney cancer cells stably expressing the Hsp90α/p23 or Hsp90β/p23 split RL reporters (Chan et al., (2008) Cancer Res. 68: 216-226, incorporated herein by reference in its entirety) were plated in each well of the 384-well plate and allowed to attach for 24 h. Baseline bioluminescence (BLI) signals at time 0 h were determined after addition of the RL substrate ENDUREN®. LOPAC compounds were then added and BLI signals were determined at 24 h and normalized to that of time 0 h. The known Hsp90 inhibitor PU-H71 (100 nM) (Llauger et al., (2005) J. Med. Chem. 48: 2892-2905) was the positive control. Twenty compounds with greater than 50% inhibition of BLI signals relative to carrier control treated cells were identified (FIG. 9A). The initial screen demonstrated that the assay conditions were optimal for discovery of novel potent and isoform-selective Hsp90(α/β)/p23 modulators in intact cells.

Novel Hsp90 inhibitors using a commercial 30,176 small molecule chemical compound library with unknown targets and molecular mechanisms (FIG. 9B) were also identified. 293T cells stably expressing Hsp90α/p23 or Hsp90β/p23 split RL reporters were treated with each compound at 8.3 μM. IC₅₀ values were determined by 8 point dose response curves (0.156-20 μM) for the 317 compounds that led to greater than 45% inhibition of RL activities, relative to carrier control treated cells.

Inhibition of cell proliferation at 20 μM was determined by cell-titer blue assays and toxic compounds were eliminated. The IC₅₀ values for inhibition of bioluminescence signals (BLI) in cells stably expressing Hsp90(α/β)/p23 split RL reporters and the inhibition of cell proliferation for the top 19 compounds are shown in Table 1, Example 15, and their structures are shown in FIGS. 1A-1D. Compounds that acted as non-specific RL inhibitors (greater than 20% inhibition relative to carrier control treated cells), as assessed by 293T cells stably expressing full length RL were eliminated (FIGS. 9C and 9D).

### Efficacy of Lead Compounds in Inhibition of Hsp90 Chaperone Activities:

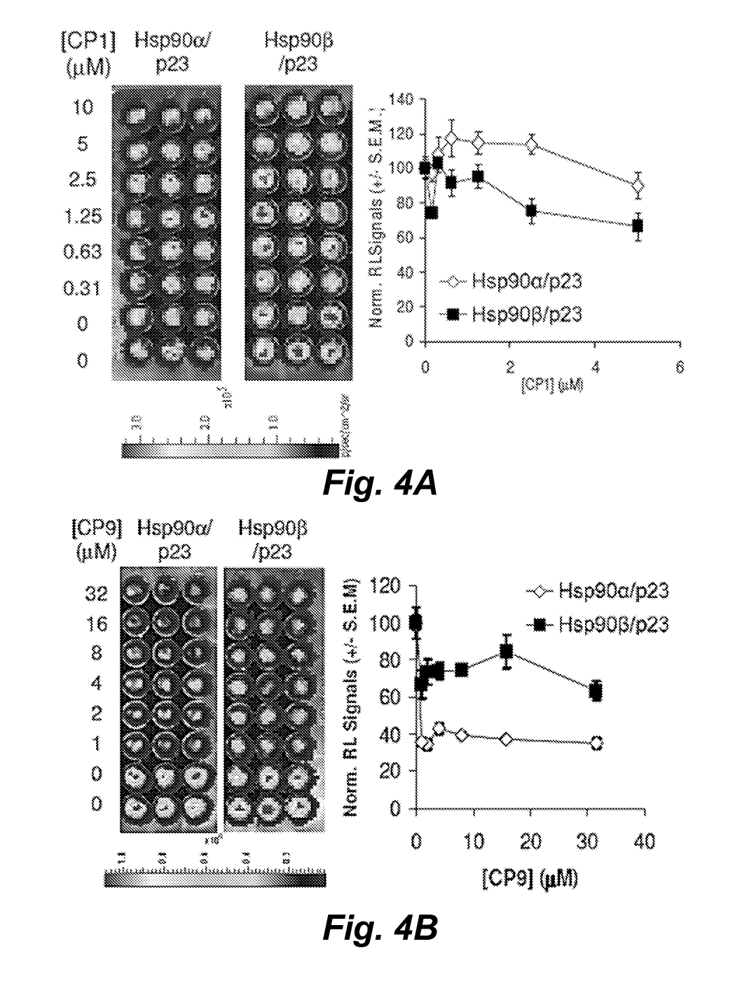

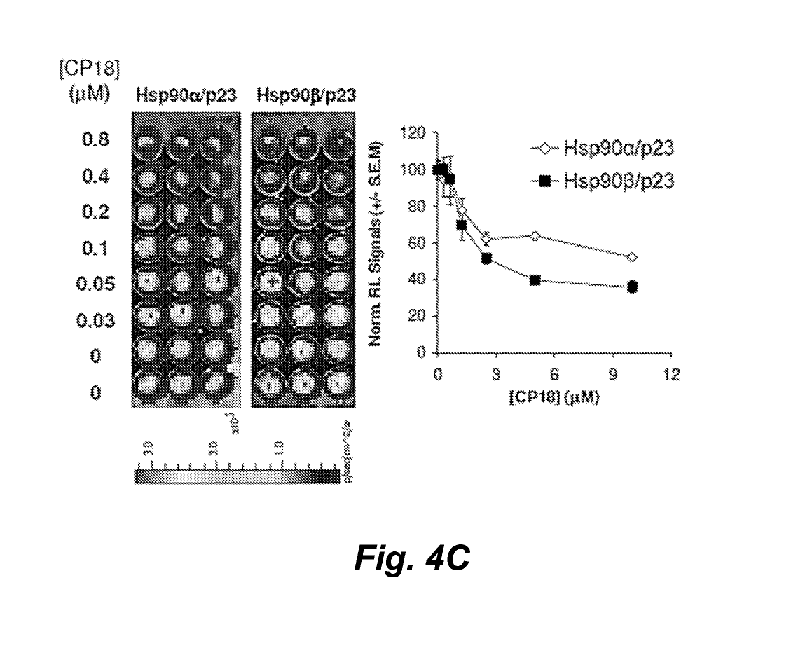

Hsp90 inhibitors lead to inhibition of Hsp90 chaperone activities and the subsequent degradation of multiple client Hsp90 proteins in cancer cells (Solit & Chiosis (2008) Drug Discovery Today 13: 38-43; Solit & Rosen (2006) Curr. Top. Medicin. Chem. 6: 1205-1214; Workman & Powers (2007) Nat. Chem. Biol. 3: 455-457). Two compounds with the greatest inhibition of Hsp90α/p23 BLI signals (CP1 and CP18) and CP9 (N-(5-methylisoxazol-3-yl)-2-[4-(thiophen-2-yl)-6-(trifluoromethyl)pyrimidin-2-ylthio]acetamide) that was more selective for inhibition of Hsp90α/p23 BLI signals, relative to that of Hsp90β/p23 interactions (FIGS. 4A-4C), were examined further.

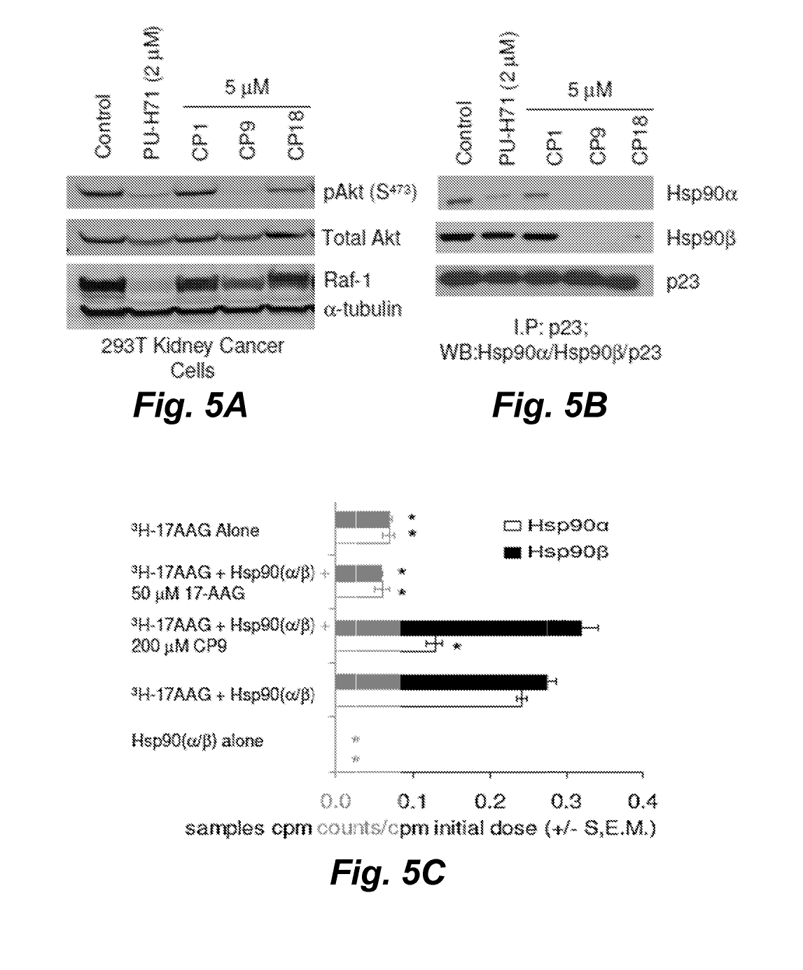

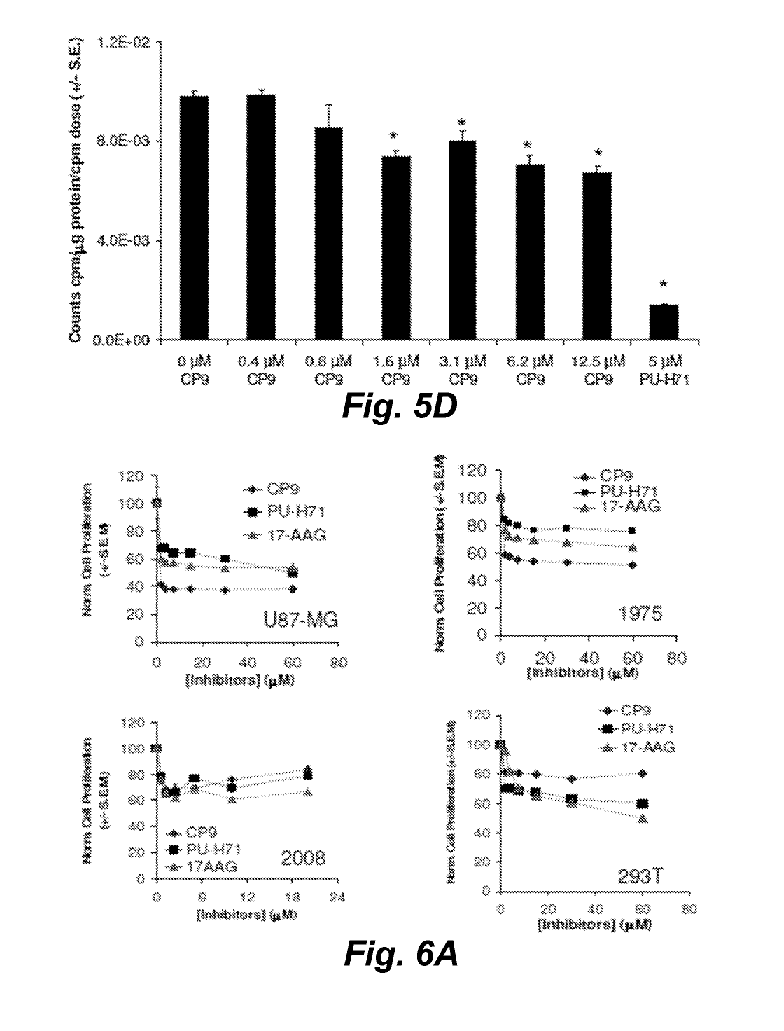

To determine if these compounds inhibited Hsp90 chaperone activities, 293T cells were treated with CP1, CP9 and CP18 (5 μM) for 24 h and the expression of phosphorylated (pAkt)/total Akt and Raf-1 were determined by western blotting (FIG. 5A). Cells treated with PU-H71 (2 μM) served as a positive control. CP1 did not significantly decrease the levels of all three Hsp90 client proteins, while CP9 was more effective than CP18 on degradation of phosphorylated/total Akt and Raf-1 (FIG. 5A). PU-H71 decreased levels of Raf-1, pAkt and total Akt, as expected. The disruption of endogenous Hsp90(α/β)/p23 interactions by CP9 and CP18 was also confirmed by co-immunoprecipitation (FIG. 5B). Based on these results, efforts were focused on further characterization of CP9.

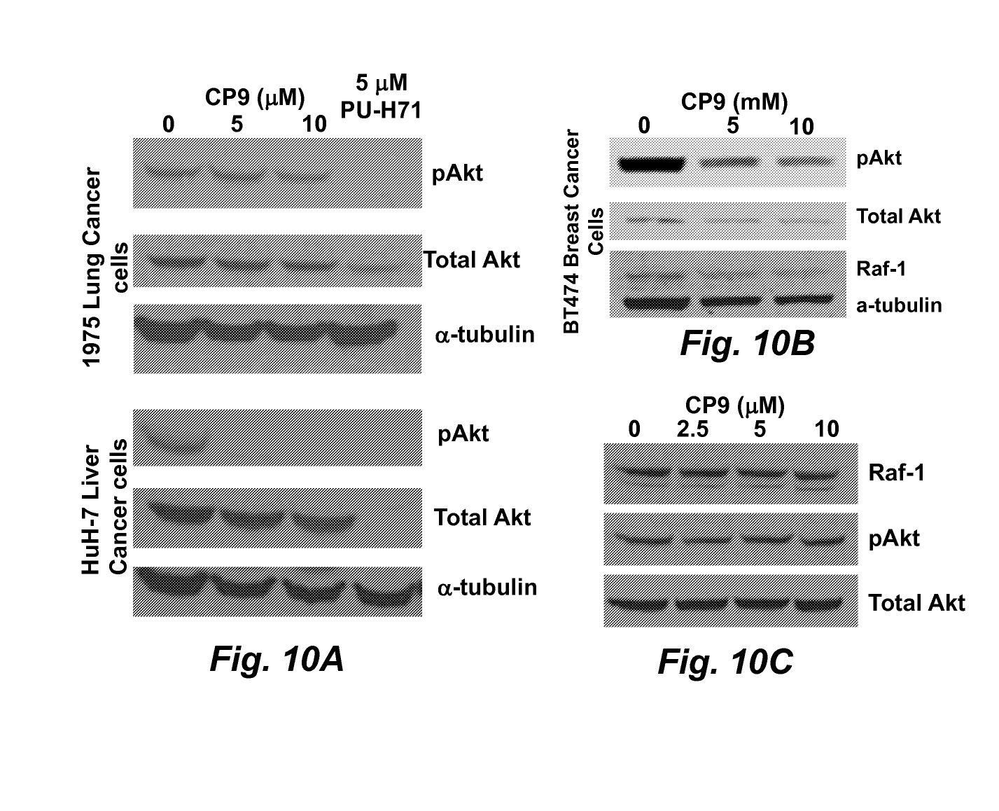

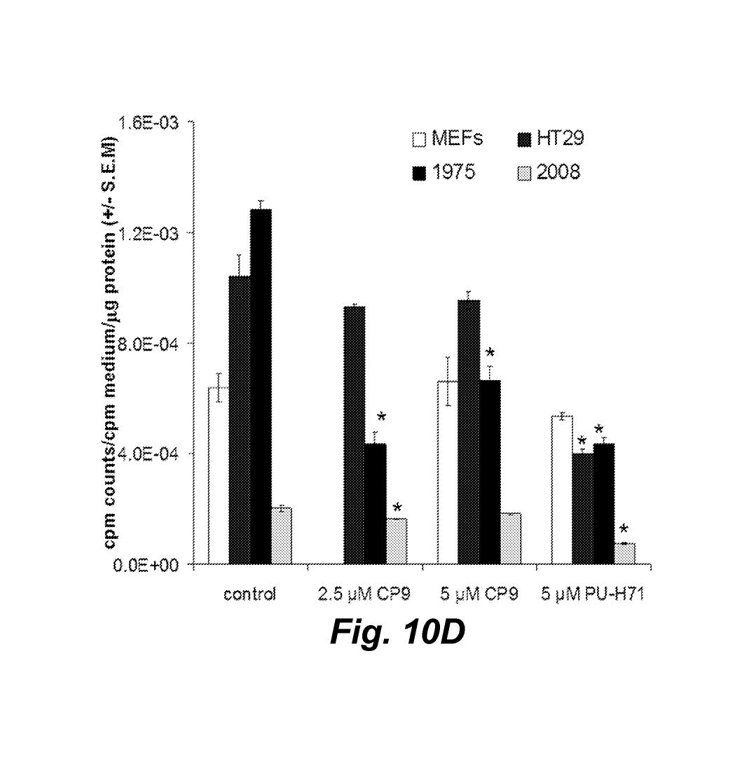

CP9 led to various levels of degradation of phosphorylated/total Akt and Raf-1 in multiple cancer cell lines, including breast (BT474/MCF-7/SK-Br3) (FIG. 10B), lung (1975) (FIG. 10B), fibrosarcoma (HT1080), liver (4-4/HUH7) (FIG. 10A), prostate (PC3), ovarian (2008), glioblastoma (U87MG) and colon (HT29) (FIG. 10A), using PU-H71 as a positive control. CP9 had no effect on the expression of the Hsp90 client proteins in normal mouse embryonic fibroblasts (MEF) cells (FIG. 10C).

To confirm the direct binding of CP9 to Hsp90(α/β), an in vitro competitive binding assay using purified Hsp90α and Hsp90β was performed using radiolabeled Hsp90 inhibitor ³H-17AAG. Hsp90 proteins were pre-bound with CP9 or cold 17-AAG as a control prior to incubation with ³H-17AAG. Unbound ³H-17AAG was removed and the amount of ³H-17AAG bound to the Hsp90 proteins was determined. FIG. 5C shows that CP9 reduced the binding of ³H-17AAG to purified Hsp90α by about 50% (p<0.05), but did not significantly affect the binding to Hsp90β. To confirm cellular Hsp90 as the target of CP9, uptake studies in intact HT29 cells were performed by monitoring the competitive binding of ³H-17AAG. CP9 binding to Hsp90 (either in the same N-terminal ATP pocket or other portions of Hsp90 that are indispensable for binding of 17-AAG) reduces ³H-17AAG binding to cellular Hsp90. PU-H71 was used as a positive control. FIG. 5D shows that CP9 led to a dose-dependent decrease in uptake of ³H-17AAG, with a maximum of 30% reduction relative to carrier control treated cells (p <0.05) (FIG. 5D). Accordingly, employing the imaging assay for HTS of the disclosure, a novel Hsp90 inhibitor (CP9) that binds to Hsp90 was identified. CP9 leads to the selective disruption of Hsp90α//p23 interactions and subsequently resulting in degradation of Hsp90 client proteins.

### CP9 Inhibits Cell Proliferation, Glucose Metabolism and Mammalian Thymidine Kinase Activities in Multiple Cancer Cell Lines

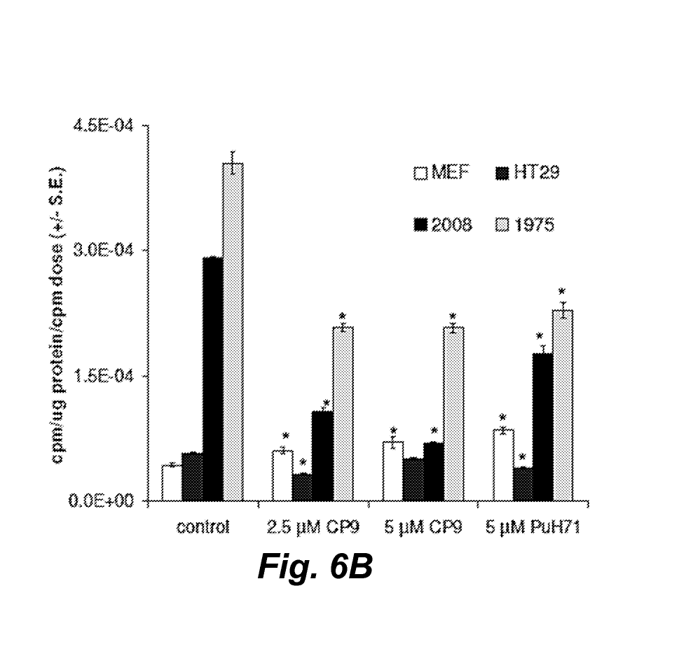

To determine if CP9 also inhibited cell proliferation, 2008, 293T, U87-MG, and 1975 cells were treated with different concentrations of CP9 and cell proliferation was monitored by ALAMAR BLUE® assay. FIG. 6A shows that CP9 was more effective than PU-H71 and 17-AAG in inhibition of U87-MG and 1975 cell proliferation but similar or less effective than them in 293T and 2008 cells. CP9 had no effect on proliferation of normal MEFs. Since CP9 led to decrease in phosphorylated Akt (FIGS. 5A and 10A-10C), it was expected to inhibit glucose metabolism through relief of inhibition of glycogen synthase kinase 313 (Nair & Olanow (2008) J. Biol. Chem. M707238200; Dandekar et al., (2007) J. Nucl. Med. 48: 602-607).

Inhibition of glucose metabolism by CP9 in cell culture was monitored by cell uptake studies with ³H-fluoro-deoxyglucose (³H-FDG) as described in Dandekar et al. ((2007) J. Nucl. Med. 48: 602-607) as shown in FIG. 6B. PU-H71 was used as a positive control.

To determine the specificity of CP9 in inhibition of glucose metabolism, normal MEF cells were used as a control. FIG. 6B shows that CP9 decreased ³H-FDG uptake in the cancer cells, without significantly affecting that of MEFs. Likewise, CP9 inhibits ³H-FLT uptake (a surrogate for in mammalian thymidine kinase activities) in all 3 cancer cell lines, but not in normal MEFs cells (FIG. 10C). Collectively, the data show that CP9 specifically degrades Hsp90 client proteins and inhibits glucose metabolism, thymidine kinase activities and cell proliferation in cancer cells.

### Non-Invasive Monitoring of Disruption of Hsp90(α/β)/p23 Interactions in Living Mice by CP9:

To monitor the inhibition of Hsp90(α/β)/p23 interactions by CP9 in living mice, a second reporter (FL-eGFP) was introduced into the 293T cells stably expressing Hsp90(α/β)/p23 split RL reporters (now referred as 293T(α/β)—FG cells). Although ¹⁸F-FLT has also been used to monitor tumor cell proliferation in small animals by PET (Tseng et al., (2005) J. Nucl. Med. 46: 1851-1857), FL imaging was used because of the higher sensitivity and ease of performing sequentially with RL imaging for Hsp90(α/β)/p23 interactions. This is feasible since the RL substrate coelentarazine (cltz) and the FL substrate D-Luciferin (D-Luc) do not cross react (Bhaumik et al., (2004) J. Biomed Optics 9: 578-586). Baseline RL and FL signals in each implanted tumor were determined upon i.v. substrate injection.

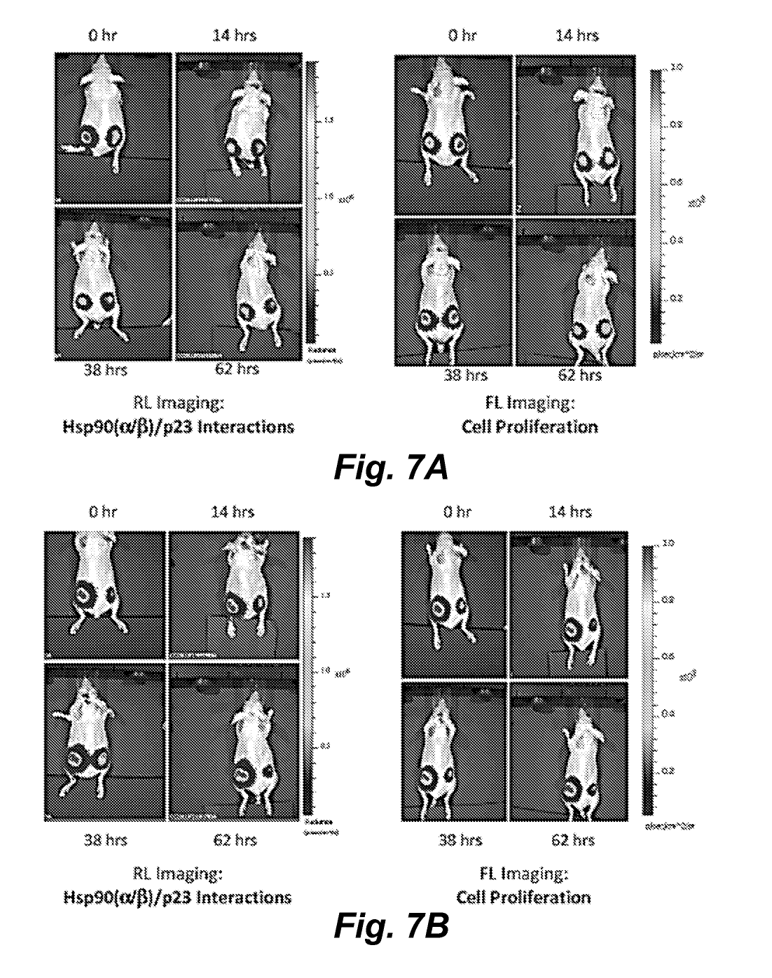

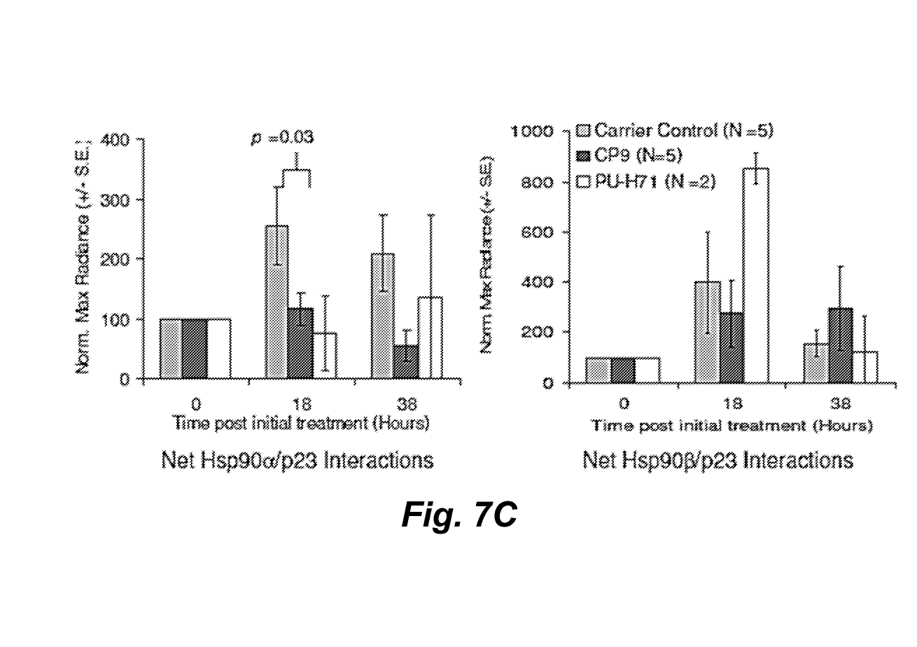

Based on experience with the Hsp90 inhibitors 17-AAG and PU-H71, CP9 (80 mg/kg) (n=5) was tested in animal experiments by i.p. injection with four doses at 16, 24, 49 h post baseline imaging (FIG. 7A). An equal volume of DMSO was injected into the carrier control group (n=5, FIG. 7B). Mice treated with 75 mg/kg PU-H71 (n=2) served as positive controls. Mice were re-imaged for Hsp90(α/β)/p23 interactions and cell proliferation via RL (FIG. 5B, left panels) and FL (FIG. 5B, right panels) imaging at the time points indicated, respectively. To account for the effect of cell number on Hsp90(α/β)/p23 interactions, RL signals were normalized to FL signals for each tumor at each time point, prior to normalization to that of time 0 h.

CP9 led to inhibition of RL signals in tumors bearing Hsp90α/p23 (left flank) and Hsp90β/p23 (right flank) xenografts, relative to time 0 h, as shown in FIG. 7A. Normalization of RL signals for cell number via FL imaging shows that RL/FL ratios in Hsp90α/p23 xenografts in CP9 treated mice at 14 and 38 h were 122±39% and 79±19%, respectively (FIG. 7C). On the other hand, the RL/FL ratios in Hsp90α/p23 xenografts treated with carrier control at 14 and 38 h were 182±22% and 132±18%, respectively (p<0.05 at 38 hrs vs. mice treated with CP9). Thus CP9 inhibits Hsp90α/p23 interactions in tumor xenografts.

RL/FL ratios in Hsp90β/p23 xenografts in CP9 treated mice at 14 h and 38 h were 58±18% and 83±42%, respectively. On the other hand, the RL/FL ratios in Hsp90β/p23 xenografts treated with carrier control at 14 h and 38 h were 130±49% and 194±96%, respectively (p>0.05 at both time points vs. mice treated with CP9). Thus CP9 was less effective in inhibition of Hsp90β/p23 interactions, compared to that of Hsp90α/p23 interactions. At 62 h post-CP9 treatment, the RL/FL ratios in Hsp90(α/β)/p23 xenografts were similar to that of carrier control treated mice (p>0.05). The data are consistent with the selectivity of CP9 in binding to Hsp90α and inhibition of Hsp90α/p23 BLI signals in cell culture, relative to Hsp90β/p23.

### CP9 Inhibits Glucose Metabolism in 293T Xenografts as Shown By ¹⁸F-FDG Small Animal Positron Emission Tomography (PET) Imaging:

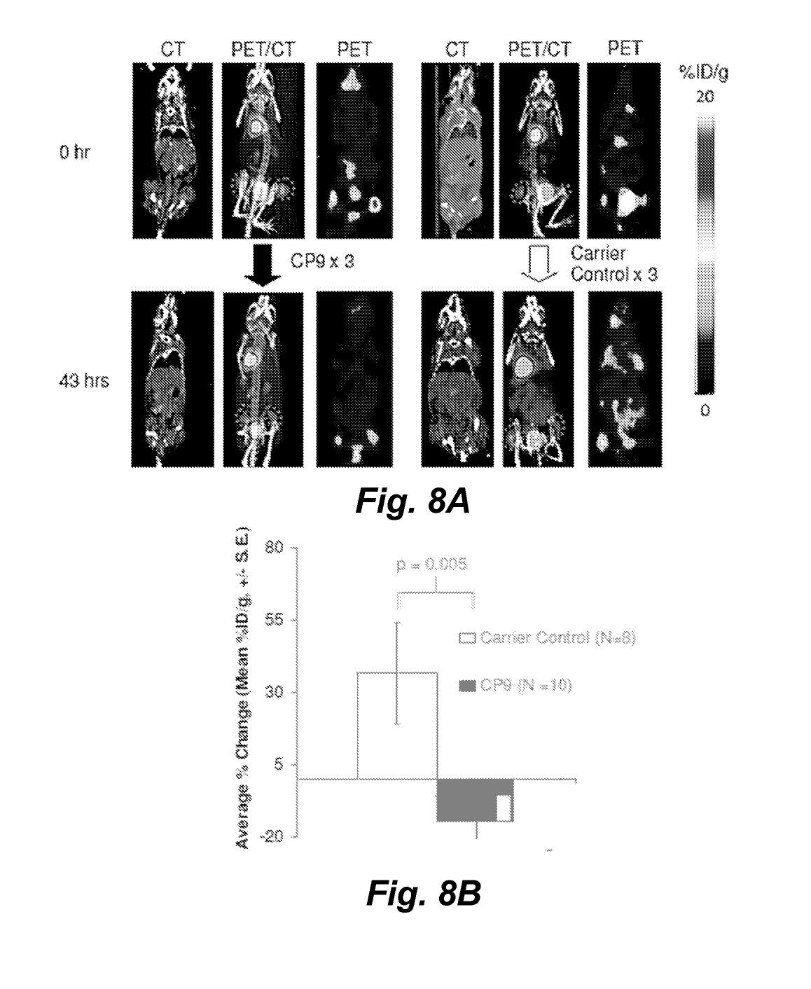

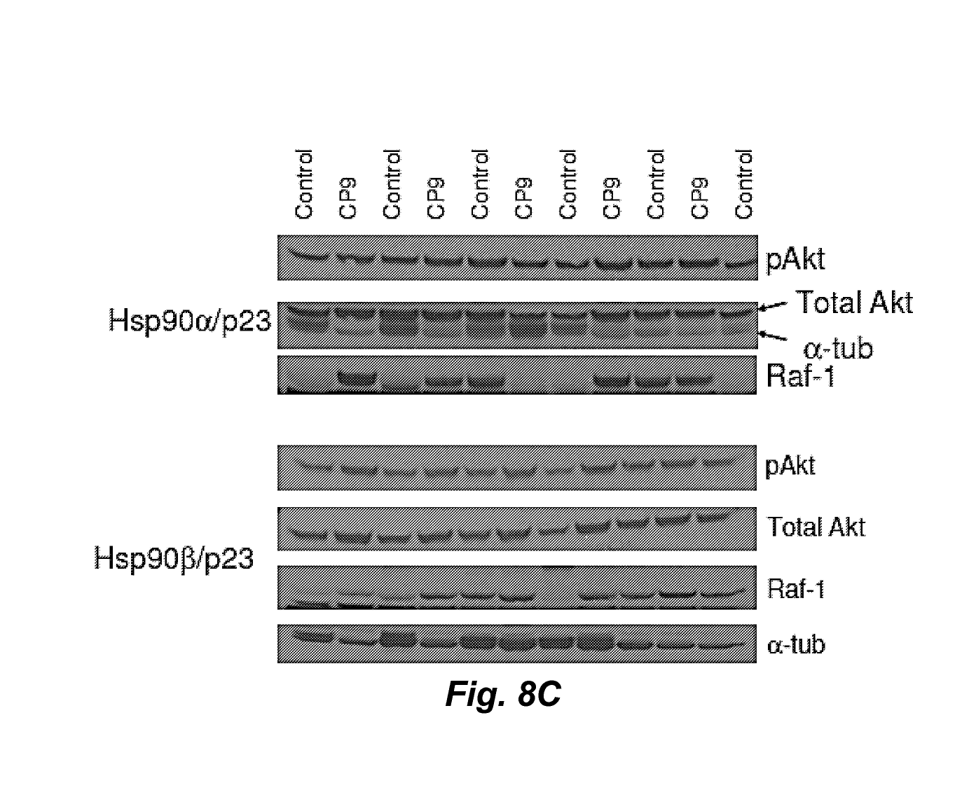

¹⁸F-FDG PET/CT has been used for repetitive and non-invasive monitoring of chemotherapy responses both in small animals and in humans (Aliaga et al., (2007) Mol. Imaging. Biol. 9: 144-150; de Geus-Oei et al., (2009) J. Nucl. Med. 50(Suppl 1): 43S-54S). Since CP9 inhibits glucose metabolism in cancer cells (FIG. 6B), its effect in the same cohort of living mice used for BLI (FIGS. 7A-7C) was monitored by small animal PET/CT imaging using ¹⁸F-FDG. The short radioactive half-life (110 mins) of ¹⁸F-FDG also permits repetitive imaging pre- and post-CP9 treatment (Santos-Oliveira & Antunes (2011) J. Nucl. Med. Technol. 39: 237-239). Baseline ¹⁸F-FDG uptake in tumor-bearing mice was determined pre-treatment with four doses of carrier control (DMSO) or CP9 (80 mg/kg in DMSO) (FIG. 8A). The % max ID/g of ¹⁸F-FDG uptake was determined by the OSEM2D method upon normalization of injected dose (Wiant et al., (2010) Med. Phys. 37: 1169-1182). The % increase in ¹⁸F-FDG uptake at 43 h relative to 0 h was determined for each tumor site.

In carrier control treated mice, ¹⁸F-FDG uptake in 293T tumors expressing Hsp90(α/β)/p23 RL reporters (n=8) increased by 37±18%, as shown in FIG. 8B. On the other hand, ¹⁸F-FDG uptake in CP9 treated tumors (n=10) decreased by 16±9% (p<0.005 relative to carrier control treated mice). Therefore, CP9 inhibits Hsp90(α/β)/p23 interactions and glucose metabolisms in tumor xenografts in living mice.

The ¹⁸F-FDG uptake in the brains of mice using CT images to delineate boundaries was also analyzed. Relative to day 0, the maximum % ID/g of ¹⁸F-FDG uptake was 114±11% in mice treated with carrier and 99±4% in mice treated with CP9. There was no statistical difference between the two groups (P>0.05). Furthermore, there were no significant decreases in weight in CP9-treated mice compared with carrier control-treated mice at 43 h (P>0.05). Thus, the data do not indicate that CP9 poses significant toxicity in mice.

### Ex Vivo Analyses for the Efficacy of CP9 in Degradation of Hsp90 Client Proteins:

To determine if the inhibition of Hsp90(α/β)/p23 interactions also leads to degradation of Hsp90 client proteins in mice, tumors were excised from sacrificed mice after the last PET/CT imaging time point. The expression of phosphorylated/total Akt and Raf-1 in tumor lysates was determined by western blotting. α-tubulin was used as a loading control.

FIG. 8C shows that CP9 treatment did not lead to significant degradation of Hsp90 client proteins, relative to carrier control treated mice (p>0.05). This observation is consistent with the imaging results at 62 h post CP9 treatment, which did not show any significant differences in Hsp90(α/β)/p23 interactions in CP9-treated and carrier control-treated mice (FIG. 7C). This may be due to insufficient drug accumulation and/or drug metabolism of CP9.

### Evaluation of the Efficacy of the Structural Analogues of CP9 in Disruption of Hsp90(α/β)/p23 Interactions and Degradation of Hsp90 Client Proteins:

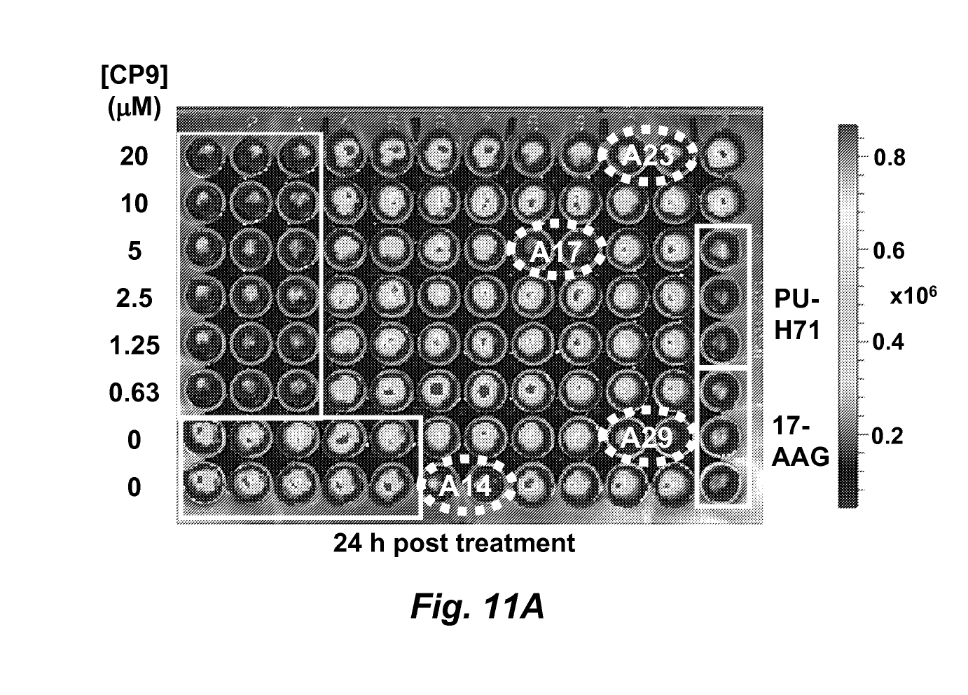

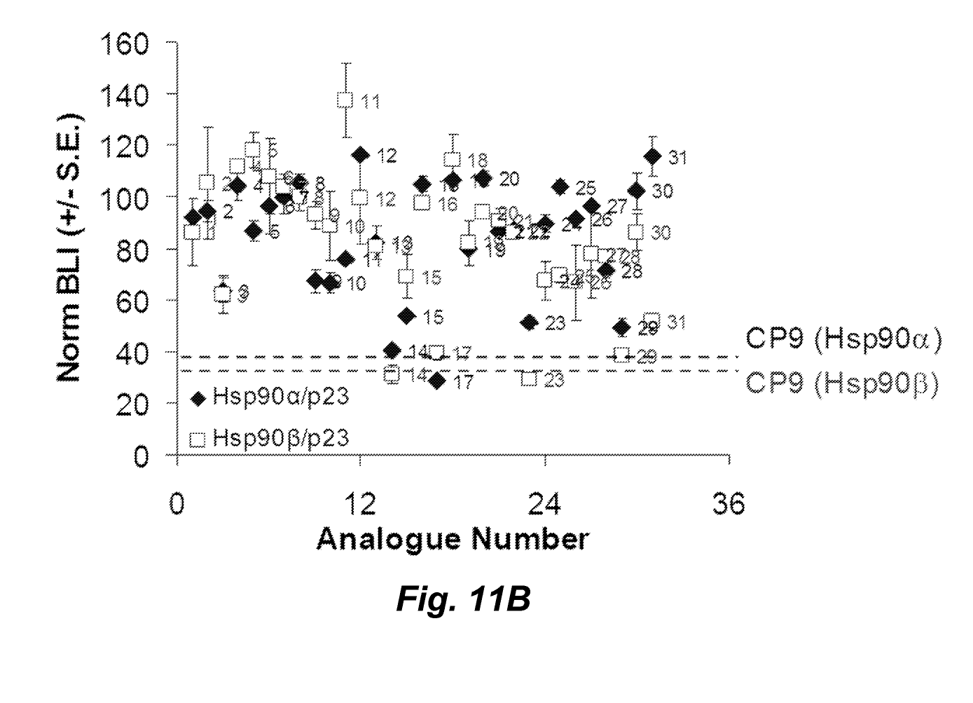

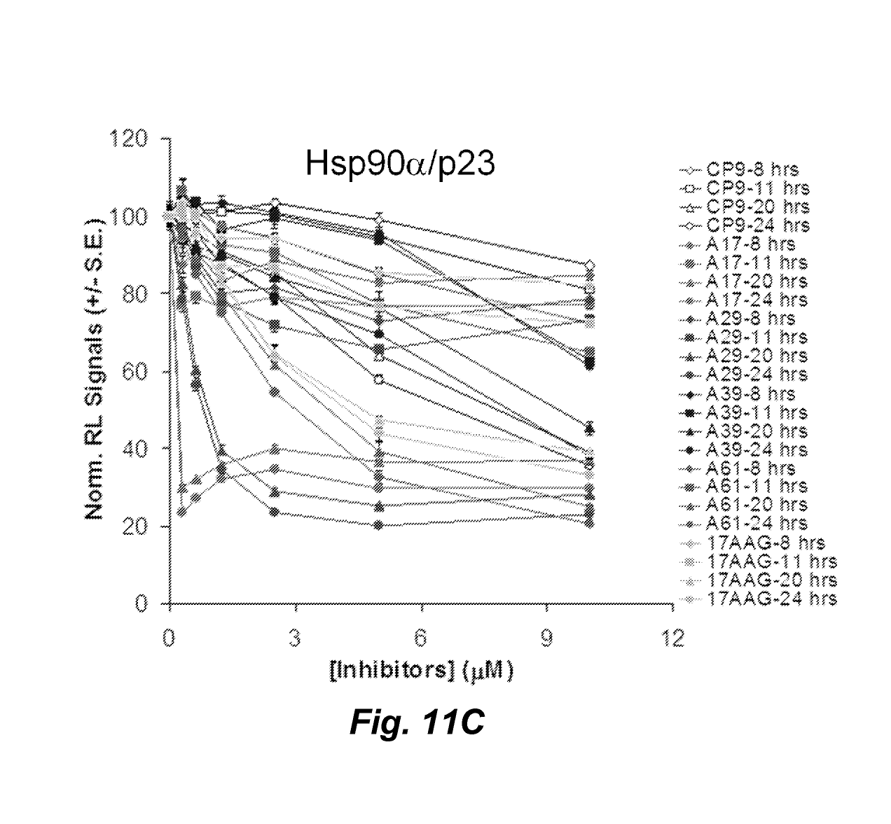

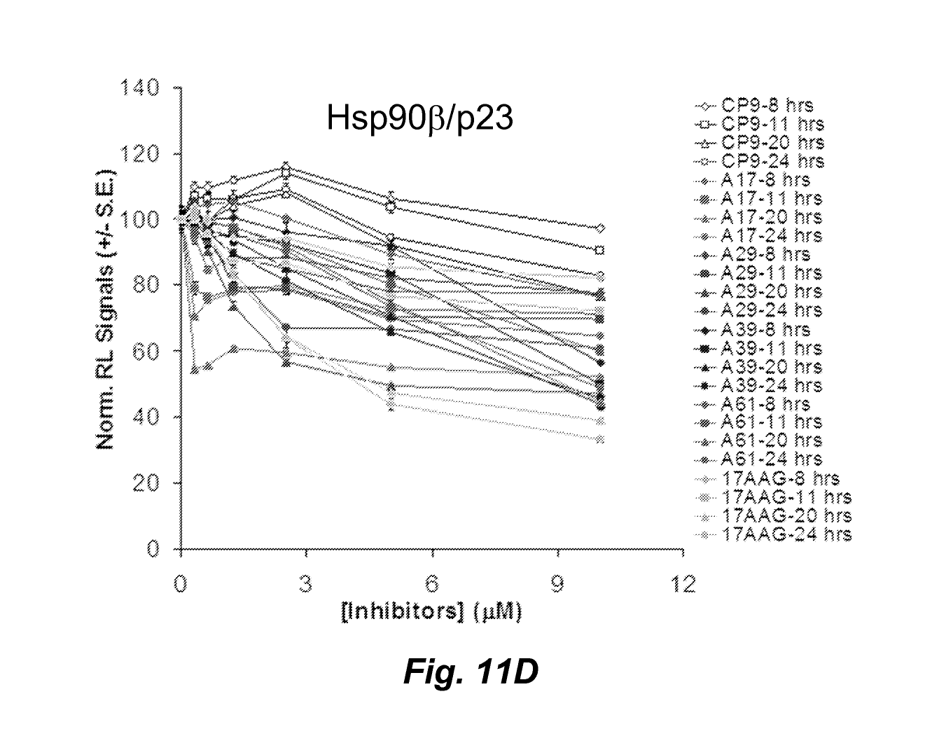

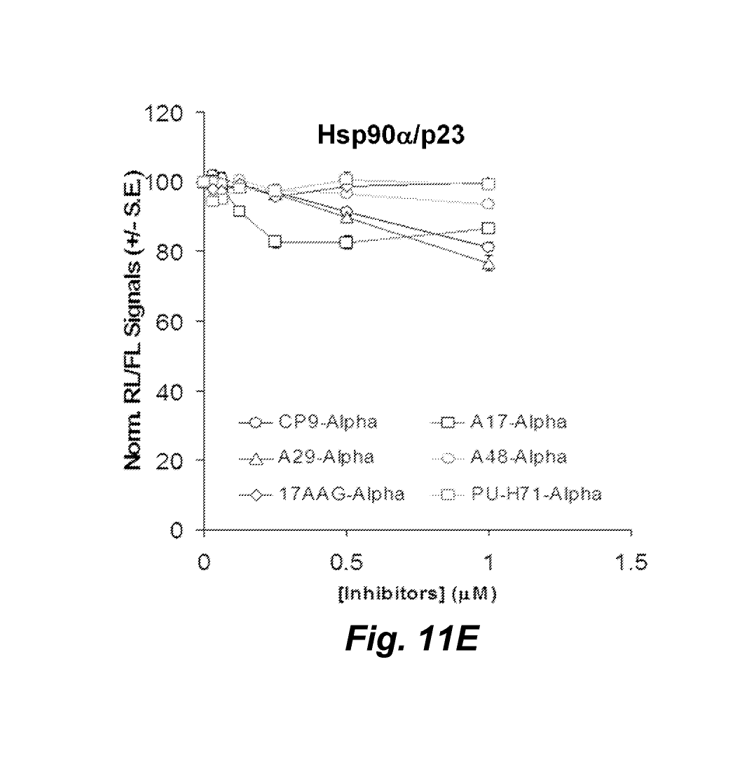

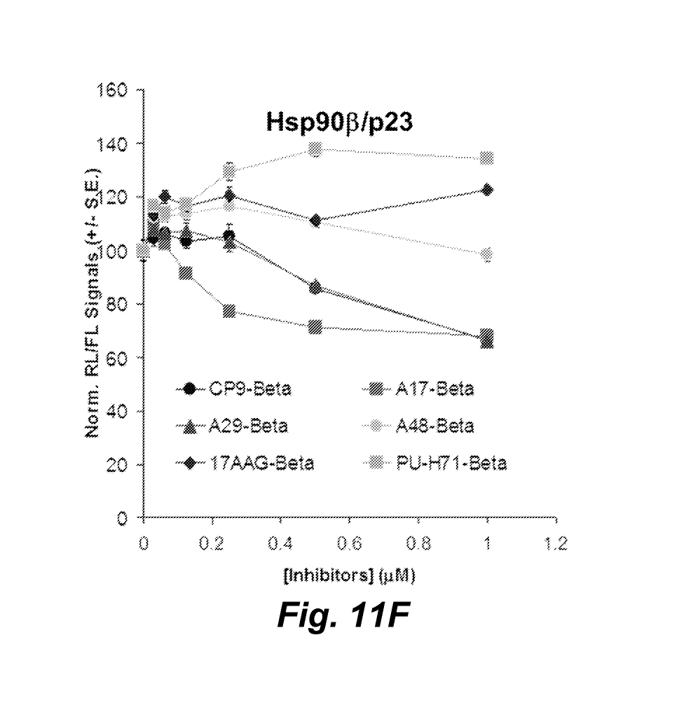

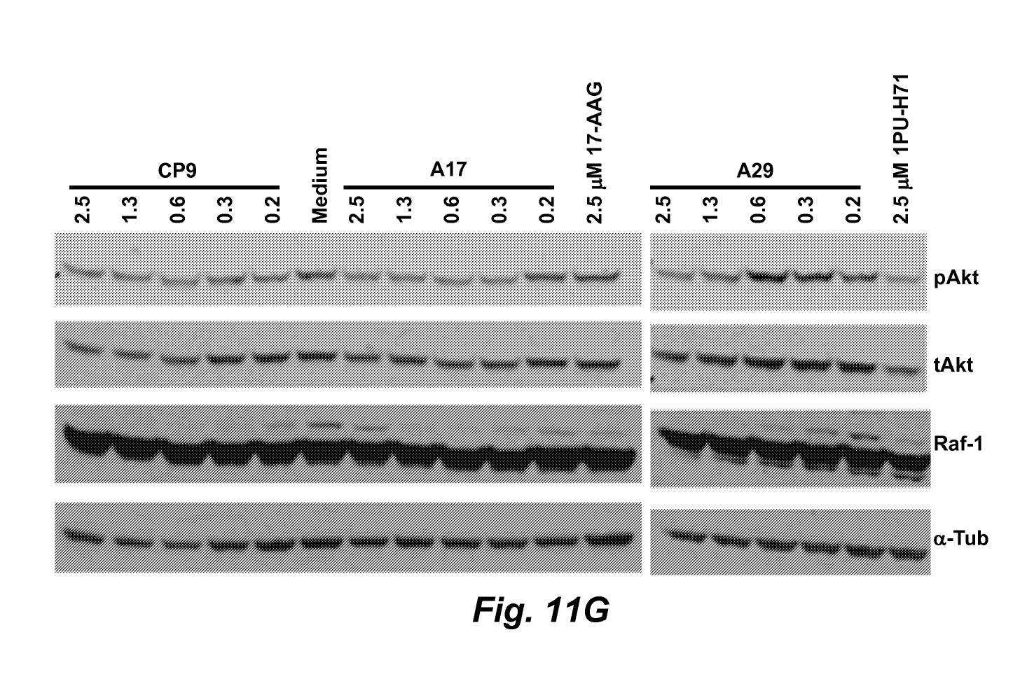

To improve the efficacy of CP9 in disruption of Hsp90(α/β)/p23 interactions in living mice, a structure-activity relationship (SAR) study was performed using 62 structural analogues (10 μM) with different modifications on CP9, as shown in FIGS. 2A-2G, using the 293T(α/β)-FG stable cells. Their effects on Hsp90(α/β)/p23 interactions and cell proliferation were monitored by sequential RL (FIG. 11A) and FL imaging at 24 h. CP9, PU-H71 and 17-AAG were used as positive controls. The net effect on Hsp90(α/β)/p23 interactions was determined by normalizing RL to that of FL signals and to that of carrier control-treated cells (FIG. 11B). Compared to the parental compound CP9 (10 μM), the analogues A14 and A17 led to similar inhibition of Hsp90α/p23 (FIG. 11B, diamonds) and Hsp90β/p23 (FIG. 11B, squares) interactions.

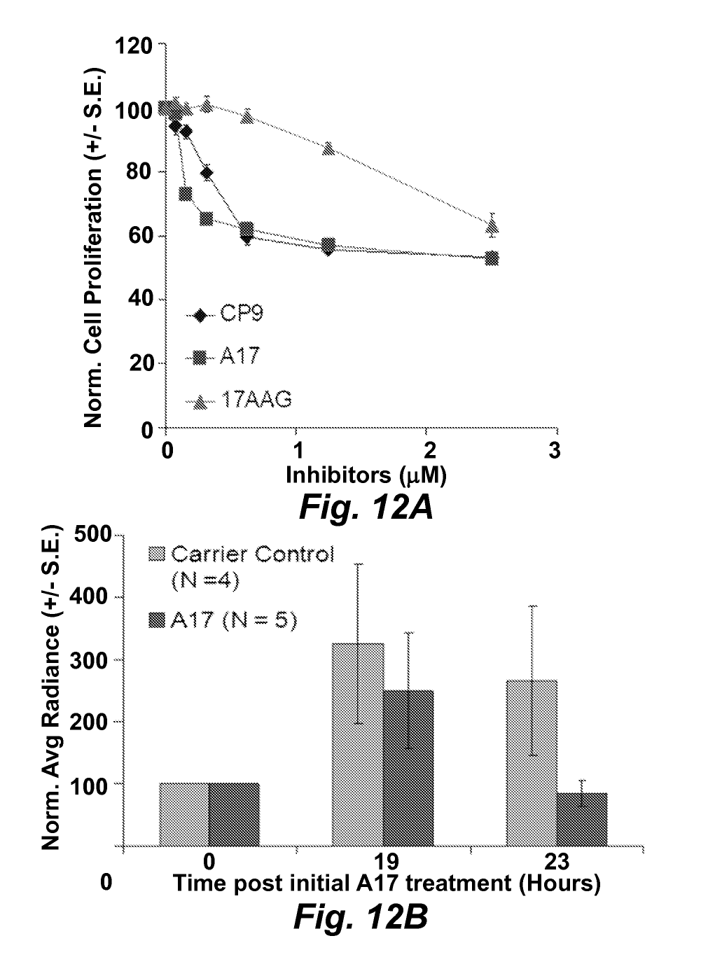

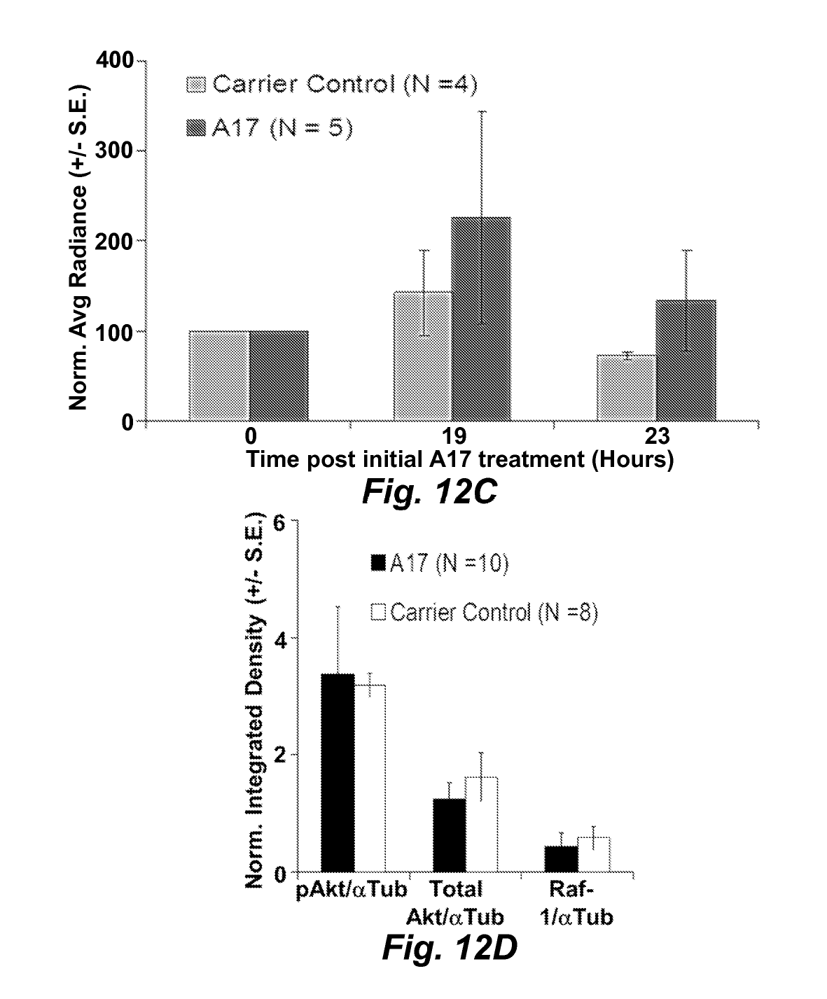

Time- and dose-response curves were established for the top 13 analogues, as shown in FIGS. 11C and 11D. Analogs A17, A29 and A61 were more effective than CP9 in inhibition of Hsp90(α/β)/p23 interactions. Analogue A17 had a 3-fold reduction in IC₅₀ for inhibition of Hsp90α/p23 signals compared to that of CP9 (0.15 μM vs. 0.45 μM). The IC₅₀ for growth inhibition by A17 was about 2-fold lower than that of CP9 (FIG. 12A).

When derivatives of CP9 with different R³ substitutions were compared (A1-16, A18-31, A35, A37, A39, A42-44, A46-47, A49, A51, A53, A55-56, A59-62), it was found that this position required an aromatic moiety and aliphatic substitution diminished efficacies in disruption of Hsp90(α/β)/p23 interactions. Furthermore, single small ortho-substitutions on the aromatic ring (as for A14, A23) and methyl substitution in meta-position (A29, A61) were tolerated. CP9 analogues with various R¹ substitutions (A17, A45, A48, A50 and A57) were also compared: a five-membered aromatic ring is likely required for binding; analogues containing phenyl rings with various substitutions in R¹ position exhibited lower activity. Only replacement of the thiophen moiety in CP9 by a furanyl substitution (A17) was tolerated and actually led to higher potency than for the parental compound. Methyl substitution in R² position was advantageous: A34 and A50 tend to have higher affinity than A40 and A45, respectively.

To determine if the reduction in IC₅₀ for Hsp90(α/β)/p23 interactions also led to improved efficacy of A17 as an Hsp90 inhibitor, 293T-FG cells were treated with different concentrations of CP9 and A17. PU-H71 and 17AAG were used as positive controls. The expression of pAkt/total Akt and Raf-1 was determined by western blotting. A17 was more effective than CP9 in degrading pAkt/total Akt and Raf-1 (FIG. 11G). Since A17, A29 and other CP9 analogues inhibit (Hsp90(a/b)/p23 interactions) and lead to degradation of Hsp90 client proteins, CP9 and its analogues were shown to function as Hsp90 inhibitors. Since A17 has a lower logP value (partition coefficient) than CP9 (2.8 versus 4.1), it may be more hydrophilic, have less non-specific binding to serum proteins and thus better bioavailability in tumors.

The efficacy of A17 in disruption of Hsp90(α/β)/p23 interactions and inhibition of cellular proliferation in living mice was monitored by BLI using the same dosing regimen as CP9. In contrast to cell culture results, A17 failed to significantly decrease Hsp90(α/β)/p23 interactions in tumor xenografts, compared to carrier control mice using the same dose regimen (p>0.05 versus carrier controlled mice (FIGS. 12B and 12C)). This may be due to the bioavailability of A17 that led to insufficient intratumoral concentration for inhibition of Hsp90(α/β)/p23 interactions (FIG. 12B). However, a SAR study platform has been advantageously established that allows rapid screening and evaluation of more potent CP9 analogues first in cell culture and subsequently in living mice.

The methods of the disclosure have coupled multimodality molecular imaging into HTS and discovered a novel class of 2-((6-(trifluoromethyl)pyrimidin-2-yl)thio)acetamide-based Hsp90 inhibitors. A dual RL/FL reporter system was utilized for monitoring isoform-selective Hsp90(α/β)/p23 interactions and cell proliferation in intact cells. Hsp90 was confirmed as the binding target of the lead compound CP9 and its efficacies as an Hsp90 inhibitor was examined. The inhibitory effects of CP9 on Hsp90(α/β)/p23 interactions and glucose metabolism in tumor xenografts in living mice were also non-invasively and repetitively monitored. Finally, SAR studies identified a more potent analogue of CP9 (A17).

CP9 as a Novel 2-((6-(trifluoromethyl)pyrimidin-2-yl)thio)acetamide-Based Hsp90 Inhibitor:

CP9 competes with ³H-17AAG for binding to purified Hsp90(α/β), reduces uptake of ³H-17AAG in intact HT29 cancer cells and disrupts Hsp90(α/β)/p23 interactions in intact 293T cells (FIGS. 5A-5D). Collectively, the data indicated that CP9 binds to Hsp90 at either the same N-terminal ATP pocket as 17-AAG and/or other parts of Hsp90 that modulate ATP binding, which is required for its interaction with p23. As an Hsp90 inhibitor, CP9 treatment leads to the degradation of Hsp90 client proteins and inhibition of glucose metabolism, mammalian thymidine kinase and cell proliferation in multiple cancer cell lines. The inhibition of Hsp90 chaperone activities is specific for cancer cells as CP9 had no effect on normal mouse embryonic fibroblasts.

The small chemical library that contained CP9 has been previously utilized in bioassays that were not specifically designed for monitoring Hsp90(α/β)/p23 interactions. Targets that reported to be affected by CP9 include β-adrenergic receptor kinase 1, polypyrimidine tract-binding protein 1α, and 5-hydroxytryptamine receptor 1E. Furthermore, it was reported that CP9 inhibits proliferation of lung cancer cells, protein assembly of perinucleolar compartment, cell and kinase activities (MEK kinase, GRK2). Since Hsp90 is involved in regulation of kinases, receptors and protein binding/folding, the data of the disclosure is consistent with the downstream effects of CP9 (Zuehlke & Johnson (2010) Biopolymers 93: 211-217).

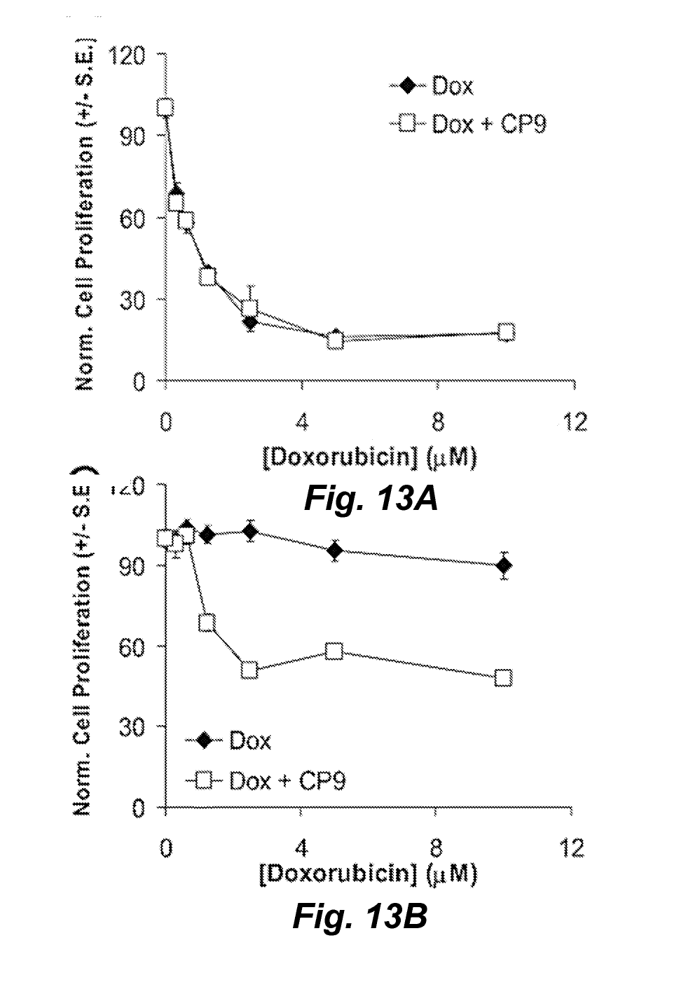

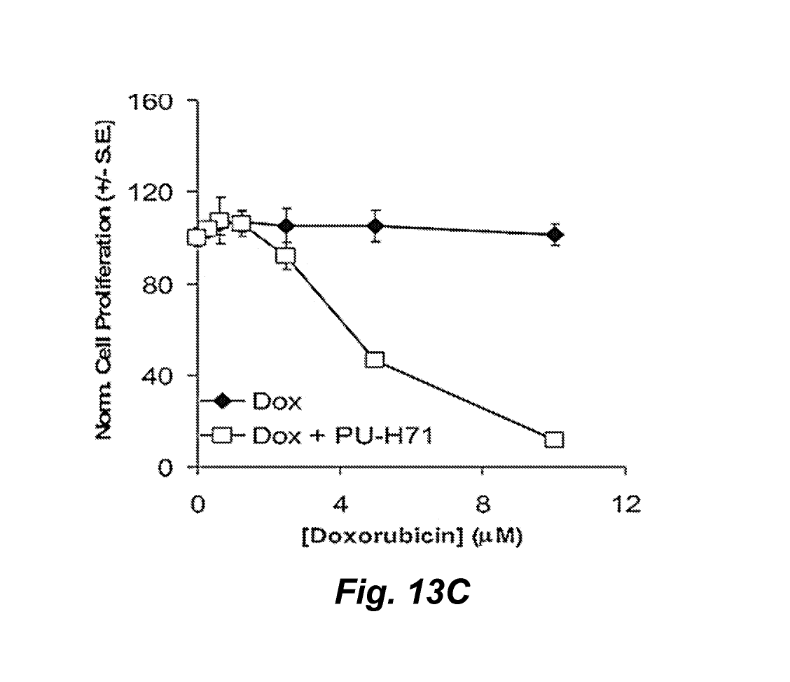

Both Hsp90 isoforms (α and β) are expressed in cancer cells, but Hsp90β is constitutively expressed, whereas the expression of Hsp90α is highly inducible during stress and drug treatment. Using the genetically encoded split Renilla reporter system according to the disclosure, it was possible to decipher the individual contributions of each isoform in determining the sensitivity of Hsp90 inhibitors. It was reported that the transcription of MDR1 is regulated by HSF-1, which in turn is regulated by Hsp90α (Trepel et al., (2010) Nat. Rev. Cancer 10: 537-549). To determine the biological significance of CP9 selectivity in inhibiting Hsp90α/p23 interactions, V79 lung cancer cells and V79/ADR cells that overexpress multidrug-resistant protein and are insensitive to growth inhibition by doxorubicin (DOX) were used. V79 and V79/ADR cells were treated with DOX alone or in the presence of CP9. In V79 cells that do not express MDR-1, the sensitivity to DOX was not affected by CP9, as shown in FIG. 13A. On the other hand, the addition of CP9 sensitized V79/ADR cells to DOX, as shown by the decrease in cell proliferation relative to treatment with DOX alone, as shown in FIG. 13B. Similar results were observed with the known Hsp90 inhibitor PU-H71 (FIG. 13C), which is more selective for disruption of Hsp90α/p23 interactions (Chan et al., (2008) Cancer Res. 68: 216-226). The results also are consistent with the observation that derivatives of Hsp90 inhibitor peptides sensitize cancer cells with overexpression of MDR to epirubicin (a class of anthracyclines that includes DOX) (Molnar et al., (2007) In Vivo 21: 429-433).

The inhibitory effects of CP9 in tumor xenografts in living mice by multimodality molecular imaging were non-invasively and repetitively monitored. BLI shows CP9 selectively inhibits Hsp90α/p23 relative to that of Hsp90β/p23 interactions (FIGS. 7A-7C), while ¹⁸F-FDG PET/CT imaging in the same cohort shows that CP9 inhibits glucose metabolism (FIGS. 8A-8C). The selective inhibition of Hsp90α/p23 interactions and reduction in glucose metabolism in mice were consistent with the cell culture results. However, CP9 did not significantly decrease the expression of the Hsp90 client proteins. The results illustrate the challenges of transitioning from cell culture to animal studies as the efficacy of any lead compound will be limited by its tumor bioavailability.

### A Unified Dual Split RL and FL Reporter System Accelerated Drug Discovery, Mechanism Validation and Lead Optimization in Living Subjects:

By introducing the FL-eGFP as a second imaging reporter, the net effect of CP9 on Hsp90(α/β)/p23 by sequential RL and FL imaging was monitored. In an attempt to improve the potency of CP9, SAR studies were performed on the 62 different structural analogues of CP9 (FIGS. 2A-2G) using the HTS dual reporter/imaging system system of the disclosure. The efficacies in degradation of Hsp90 client proteins (FIG. 11G) correspond to the inhibition of BLI signals (FIG. 11D).

The most potent analogue A17 was more effective than CP9 in inhibition of Hsp90(α/β)/p23 interactions and degradation of Hsp90 client proteins under cell culture conditions, but not in living mice (FIG. 8C). The data confirm the CP9 family as novel 2-((6-(trifluoromethyl)pyrimidin-2-yl)thio)acetamide-based Hsp90 inhibitors that were discovered by coupling HTS, SAR efforts with multimodality molecular imaging. The strategy allows rapid evaluation of structural analogues generated by medicinal chemistry with better potency and bioavailability, and significantly reduces the costs of scaling up the syntheses of compounds.

A therapeutic composition of the disclosure may comprise a carrier, such as one or more of a polymer, carbohydrate, peptide or derivative thereof, which may be directly or indirectly covalently attached to the compound. A carrier may be substituted with substituents described herein including without limitation one or more alkyl, amino, nitro, halogen, thiol, thioalkyl, sulfate, sulfonyl, sulfinyl, sulfoxide, hydroxyl groups. In aspects of the disclosure the carrier is an amino acid including alanine, glycine, praline, methionine, serine, threonine, asparagine, alanyl-alanyl, prolyl-methionyl, or glycyl-glycyl. A carrier can also include a molecule that targets a compound of the disclosure to a particular tissue or organ.

Compounds of the disclosure can be prepared using reactions and methods generally known to the person of ordinary skill in the art, having regard to that knowledge and the disclosure of this application including the Examples. The reactions are performed in solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

The compounds of the disclosure which are basic in nature can form a wide variety of different salts with various inorganic and organic acids. In practice is it desirable to first isolate a compound of the disclosure from a reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds of the disclosure are readily prepared by treating the base compound with a substantially equivalent amount of the chosen mineral or inorganic or organic acid in an aqueous solvent medium or in a suitable organic solvent such as methanol or ethanol. Upon careful evaporation of the solvent, the desired solid salt is obtained.

Compounds of the disclosure which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Therapeutic efficacy and toxicity of compounds, compositions and methods of the disclosure may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred. By way of example, one or more of the therapeutic effects can be demonstrated in a subject or disease model by the screening methods of the disclosure.

The disclosure provides dosage forms, formulations, and methods that provide advantages and/or beneficial pharmacokinetic profiles, more particularly sustained pharmacokinetic profiles. A compound of the disclosure can be utilized in dosage forms in pure or substantially pure form, in the form of its pharmaceutically acceptable salts, and also in other forms including anhydrous or hydrated forms.

A beneficial pharmacokinetic profile may be obtained by administering a formulation or dosage form suitable for once, twice-a-day, three-times-a-day, or more administration comprising one or more compounds of the disclosure present in an amount sufficient to provide the required concentration or dose of the compound to an environment of use to treat a disease disclosed herein, in particular a cancer.

Embodiments of the disclosure relate to a dosage form comprising one or more compounds of the disclosure that can provide peak plasma concentrations of the compound of between about 0.001 to 2 mg/ml, 0001 to 1 mg/ml, 0.0002 to 2 mg/ml, 0.005 to 2 mg/ml, 001 to 2 mg/ml, 0.05 to 2 mg/ml, 0.001 to 0.5 mg/ml, 0.002 to 1 mg/ml, 0.005 to 1 mg/ml, 0.01 to 1 mg/ml, 005 to 1 mg/ml, or 0.1 to 1 mg/ml. The disclosure also provides a formulation or dosage form comprising one or more compound of the disclosure that provides an elimination t_{1/2} of 0.5 to 20 h, 0.5 to 15 h, 0.5 to 10 h, 0.5 to 6 h, 1 to 20 h, 1 to 15 h, 1 to 10 h, or 1 to 6 h.

A subject may be treated with a compound of the disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A compound or composition may be administered to a subject for about or at least about 24 h, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

A beneficial pharmacokinetic profile can be obtained by the administration of a formulation or dosage form suitable for once-, twice-, or three-times-a-day administration, preferably twice-a-day administration comprising one or more compound of the disclosure present in an amount sufficient to provide the requited dose of the compound. The required dose of a compound of the disclosure administered once, twice, three times or more daily is about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, 1 to 3 mg/kg, 1 to 2.5 mg/kg, or less than or about 10 mg/kg, 5 mg/kg, 2.5 mg/kg, 1 mg/kg, or 0.5 mg/kg twice daily or less.

Certain dosage forms and formulations may minimize the variation between peak and trough plasma and/or brain levels of compounds of the disclosure and in particular provide a sustained therapeutically effective amount of the compounds.

The disclosure also contemplates a formulation or dosage form comprising amounts of one or more compound of the disclosure that results in therapeutically effective amounts of the compound over a dosing period, in particular a 24 h dosing period. The therapeutically effective amounts of a compound of the disclosure are between about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, or 0.1 to 1 mg/kg.

A medicament or treatment of the disclosure may comprise a unit dosage of at least one compound of the disclosure to provide therapeutic effects. A “unit dosage” or “dosage unit” refers to a unitary, i.e., a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A formulation or dosage form of the disclosure may be an immediate release dosage form or a non-immediate release delivery system, including without limitation a delayed-release or sustained-release dosage form.

The disclosure provides a sustained-release dosage form of a compound of the disclosure which advantageously achieves a more sustained drug plasma while mitigating or eliminating drug concentration spikes by providing a substantially steady release of the compound over time. A substantially constant plasma concentration preferably correlates with one or more therapeutic effects disclosed herein. In embodiments, the sustained-release dosage form is for oral administration.

A composition, in particular a dosage form or formulation, may be in any form suitable for administration to a subject, including without limitation, a form suitable for oral, parenteral, intravenous (bolus or infusion), intraperitoneal, subcutaneous, or intraffinsculas administration. A dosage form or formulation may be a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder.

A dosage form or formulation can be an oral dosage form or formulation such as tablets, caplets, soft and hard gelatin capsules, pills, powders, granules, elixirs, tinctures, suspensions, syrups, and emulsions. The dosage form or formulation can be a parenteral dosage form such as an active substance in a sterile aqueous or non-aqueous solvent, such as water, isotonic saline, isotonic glucose solution, buffer solution, or other solvents conveniently used for parenteral administration.

A compound of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21st Edition. 2005, University of the Sciences in Philadelphia (Editor), Mack Publishing Company), and in The United States Pharmacopeia: The National Formulary (USP 24 NF19) published in 1999. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically, acceptable inert carrier such as ethanol, glycerol, water, and the like. Suitable binders (e.g., gelatin, starch, corn sweeteners, natural sugars including glucose, natural and synthetic gums, and waxes), lubricants (e.g., sodium oleate, sodium stearate, magnesium stearate, sodium benzoate, sodium acetate, and sodium chloride), disintegrating agents (e.g., starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

A composition of the disclosure can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g., encapsulation in liposomes, microparticles, microcapsules, and the like.

Formulations for parenteral administration may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g., antioxidants such as methylhydroxybenzoate or similar additives.

A composition of the disclosure may be sterilized by, for example, filtration through a bacteria retaining filter, addition of sterilizing agents to the composition, irradiation of the composition, or heating the composition. Alternatively, the compounds or compositions of the present disclosure may be provided as sterile solid preparations e.g., lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

According to the disclosure, a kit is provided. In an aspect, the kit comprises a compound of the disclosure or a formulation of the disclosure in kit form. The kit can be a package which houses a container which contains compounds of the disclosure or formulations of the disclosure and also houses instructions for administering the compounds or formulations to a subject. The disclosure further relates to a commercial package comprising compounds of the disclosure or formulations of the disclosure together with instructions for simultaneous, separate or sequential use. In particular, a label may include amount, frequency, and method of administration.

The disclosure also provides a pharmaceutical pack or kit comprising one or more containers filled with one or more of the ingredients of a composition of the disclosure to provide a therapeutic effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The disclosure also relates to articles of manufacture and kits containing materials useful for treating a disease disclosed herein. An article of manufacture may comprise a container with a label. Examples of suitable containers include bottles, vials, and test tubes which may be formed from a variety of materials including glass and plastic. A container holds compounds of the disclosure or formulations of the disclosure which are effective for treating a disease disclosed herein. The label on the container indicates that the compounds of the disclosure or formulations of the disclosure are used for treating a disease disclosed herein and may also indicate directions for use. In aspects of the disclosure, a medicament or formulation in a container may comprise any of the medicaments or formulations disclosed herein.

The disclosure also contemplates kits comprising one or more compounds of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein). A medicament or formulation in a kit of the disclosure may comprise any of the formulations or compositions disclosed herein.

The compositions and methods described herein are indicated as therapeutic agents or methods either alone or in conjunction with other therapeutic agents or other forms of treatment. They may be co-administered, combined or formulated with one or more therapies or agents used to treat a condition described herein. Compositions of the disclosure may be administered concurrently, separately, or sequentially with other therapeutic agents or therapies. Therefore, compounds of the disclosure may be co-administered with one or more additional therapeutic agents for the treatment of complications resulting from or associated with a disease disclosed herein, or general medications that treat or prevent side effects.

In summary, a novel class of Hsp90 inhibitors (CP9 and its analogues) has been identified and validated by coupling molecular imaging with HTS. The work-flow allows rapid identification of cell-permeable lead compounds, followed by validation of their mechanisms and downstream effects in living mice. This will significant accelerate the next generations of therapeutics aimed at inhibiting specific chaperone proteins interactions.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

One aspect of the disclosure, therefore, encompasses embodiments of a therapeutic composition comprising an inhibitor of an Hsp90 chaperone activity, wherein the inhibitor can be selected from the group consisting of: compounds CP1-CP19 as shown in FIGS. 1A-1D and a compound having the formula I:

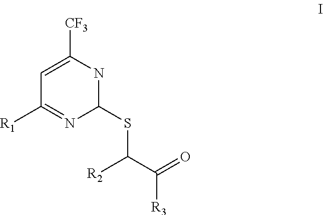

wherein R₁ can be a thiophene, a furan, a substituted or unsubstituted phenyl, or —OH; R₂ can be H or an alkyl; and R₃ can be phenylmethylamine, 4-amidopyridyl, or —NHR₄, wherein R₄ can be a substituted isoxazole, a substituted or unsubstituted alkyl, a substituted or unsubstituted branched chain alkyl, a substituted or unsubstituted —(CH)ₙPh, a substituted or unsubstituted phenyl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine, and wherein the therapeutic composition is formulated to have a dose of the inhibitor effective in reducing the viability of a cancer cell when delivered to an animal or human.

In embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In embodiments of this aspect of the disclosure, the inhibitor can have the formula I and can be selected from compounds CP9 and A1-A62 of FIGS. 2A-2G.

In some embodiments of this aspect of the disclosure, the inhibitor can have the formula I:

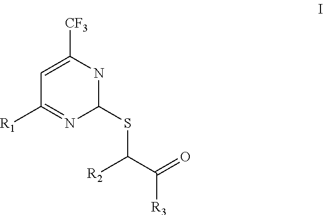

wherein R₁ can be a thiophene, a furan, phenyl, a substituted phenyl, or —OH; R₂ can be H or methyl; and R₃ can be phenylmethylamine, 4-amidopyridyl, or —NHR₄, wherein R₄ can be a substituted isoxazole, an alkyl, a branched chain alkyl, a —(CH)ₙPh, a substituted —(CH)ₙPh, -Ph, a substituted phenyl, a substituted biphenyl, a cycloalkyl, a pyridyl, —(CH)ₙpyridyl, methylfuranyl, a methyltetrahydrofuran, substituted pipenazyl, or a morpholine, and wherein n=1 or 2, and the therapeutic composition can be formulated to have a dose of the inhibitor effective in reducing the viability of a cancer cell when delivered to an animal or human.

In these embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In these embodiments of this aspect of the disclosure, the inhibitor can be selected from compounds CP9 and A1-A62 of FIGS. 2A-2G.

In some embodiments of this aspect of the disclosure, the inhibitor is N-(5-methylisoxazol-3-yl)-2-(4-(thiophen-2-yl)-6-(trifluoromethyl)pyrimidin-2-ylthio)acetamide (CP9) having the formula:

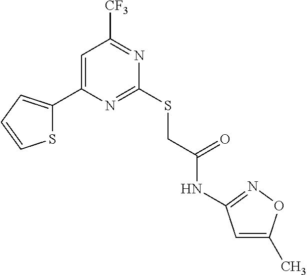 [SMILES: Cc1cc(NC(=O)CSc2nc(-c3cccs3)cc(C(F)(F)F)n2)no1]

In other embodiments of this aspect of the disclosure, the inhibitor can be CP9, A17, A29, or A61, or a combination thereof.

In the embodiments of this aspect of the disclosure, the therapeutic composition of the disclosure can further comprise a pharmaceutically acceptable carrier.

Another aspect of the disclosure encompasses embodiments of a method of reducing the viability of a cancer cell in an animal or human, the method comprising delivering to the animal or human a therapeutically effective amount of an inhibitor of an Hsp90 chaperone activity, wherein the inhibitor can be selected from the group consisting of: compounds CP1-CP19 as shown in FIGS. 1A-1D and a compound having the formula I:

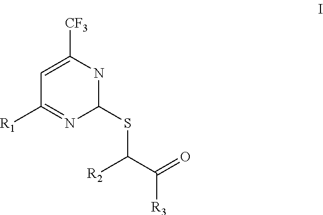

wherein R₁ can be a thiophene, a furan, a substituted or unsubstituted phenyl, or —OH; R₂ can be H or an alkyl; and R₃ can be phenylmethylamine, 4-amidopyridyl, or —NHR₄, wherein R₄ can be a substituted isoxazole, a substituted or unsubstituted alkyl, a substituted or unsubstituted branched chain alkyl, a substituted or unsubstituted —(CH)ₙPh, a substituted or unsubstituted phenyl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine.

In embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In embodiments of this aspect of the disclosure, the inhibitor can have the formula I and can be selected from compounds CP9 and A1-A62 of FIGS. 2A-2G.

In embodiments of this aspect of the disclosure, the inhibitor can have the formula I:

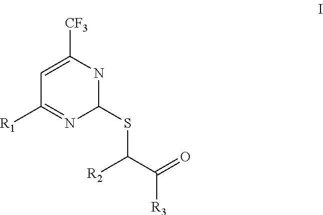

wherein R₁ can be a thiophene, a furan, phenyl, a substituted phenyl, or —OH; R₂ can be H or methyl; and R₃ can be phenylmethylamine, 4-amidopyridyl, or —NHR₄, wherein R₄ can be a substituted isoxazole, an alkyl, a branched chain alkyl, a —(CH)ₙPh, a substituted —(CH)ₙPh, -Ph, a substituted phenyl, a substituted biphenyl, a cycloalkyl, a pyridyl, —(CH)ₙpyridyl, methylfuranyl, a methyltetrahydrofuran, substituted pipenazyl, or a morpholine, and wherein n=1 or 2, and where the therapeutic composition can be formulated to have a dose of the inhibitor effective in reducing the viability of a cancer cell when delivered to an animal or human.

In these embodiments of this aspect of the disclosure, R₁ can be a thiophene, a furan, phenyl, or a substituted phenyl, wherein the substituted phenyl can be a methoxyphenyl, an halogenated phenyl, or a dimethoxyphenyl.

In some embodiments of this aspect of the disclosure, the inhibitor can be selected from compounds CP9 and A1-A62 of FIGS. 2A-2G.

In some embodiments of this aspect of the disclosure, the inhibitor is N-(5-methylisoxazol-3-yl)-2-(4-(thiophen-2-yl)-6-(trifluoromethyl)pyrimidin-2-ylthio)acetamide (CP9) having the formula:

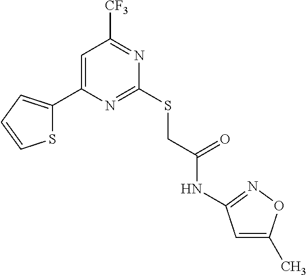 [SMILES: Cc1cc(NC(=O)CSc2nc(-c3cccs3)cc(C(F)(F)F)n2)no1]

In some embodiments of this aspect of the disclosure, the inhibitor can be CP9, A17, A29, or A61, or a combination thereof.

Yet another aspect of the disclosure encompasses embodiments of a high-throughput method for identifying an inhibitor of Heat Shock Protein 90 (Hsp90) chaperone activity, the system comprising: (a) obtaining a genetically modified cell, or progeny thereof expressing a split luciferase reporter configured to provide a detectable signal on binding of a p23 polypeptide and a Heat Shock Protein 90 (Hsp90) polypeptide in the presence of coelentarazine; (b) detecting a first detectable signal emitted from the genetically-modified cell or population thereof; (c) contacting the genetically-modified cell or progeny thereof with a compound suspected of being an Hsp90 inhibitor; (d) detecting a second detectable signal emitted from the genetically-modified cell or progeny thereof expressing the split Renilla luciferase reporter; and (e) comparing the intensities of the first and the second detectable signals, whereby if the intensity of the first detectable signal is greater than intensity of the second detectable signal, the compound is determined to inhibit the formation of a complex between p23 and an Hsp90 polypeptide.

In some embodiments of this aspect of the disclosure, the method can further comprise the steps: (f) obtaining a subject animal comprising a xenograft tumor derived from the genetically-modified cell of step (a); (g) administering to the animal coelentarazine and detecting a third detectable signal intensity from the xenograft tumor; and (h) administering to the subject animal the compound determined in step (e) to inhibit complex formation between p23 and an Hsp90 polypeptide, and coelentarazine, and obtaining a fourth detectable signal intensity from the xenograft, wherein if the fourth signal intensity is less than the third signal intensity, the compound identified in step (e) is identified as an inhibitor of complex formation between p23 and an Hsp90 polypeptide in vivo.

In some embodiments of this aspect of the disclosure, the split luciferase reporter comprises a p23 polypeptide having N-terminus fragment of a Renilla luciferase attached thereto, and an Hsp90 polypeptide having a C-terminus fragment of the Renilla luciferase attached thereto, whereby when the p23 and the Hsp90 polypeptides are in contact in the presence of ATP, the N- and C-termini of the Renilla luciferase cooperate to generate the first detectable signal in the presence of coelentarazine.

Still another aspect of the disclosure encompasses embodiments of a kit comprising a container containing a therapeutic composition comprising a compound of FIGS. 1A-1D and 2A-2G, or a pharmaceutically effective derivative thereof, and instructions for administering the compounds or formulations to a subject.

It should be emphasized that the embodiments of the present disclosure, particularly any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

### Chemicals, Enzymes and Reagents

Coelentarazine was purchased from Nanolight technology (Pinetop, Ariz.). Cell culture media, fetal bovine serum (FBS), the streptomycin/penicillin (P/S), 4-12% gradient SDS-PAGE gels were purchased from Invitrogen (Carlsbad, Calif.). Puromycin hydrochloride and 17-Allylamino-17-demethoxygeldanamycin (17-AAG) were purchased from Invivogen (San Diego, Calif.). Purine-scaffold Hsp90 inhibitor PU-H71 (Chiosis et al., (2002) Bioorgan. Medicin. Chem. 10: 3555-3564; He et al., (2006) J. Med. Chem. 49: 381-390; Zhou et al., (2004) Anal. Biochem. 331: 349-357) was dissolved as 7.65 mM PBS stock and stored at −20° C. The slow-kinetic RL substrate ENDUREN® (Promega, Madison) was dissolved in DMSO as 34 mg/ml stock and stored at −20° C.

### Example 2

### Cell Culture

All cell lines used in this study were purchased from American Type Culture Collection (Manassa, Va.) and cultured with their respective medium supplemented with 10% FBS and 1% P/S. Human 293T embryonic kidney cancer cells stably expressing Hsp90(α/β)/p23 split RL reporters as described in Chan et al., (2008) Cancer Res. 68: 216-226, incorporated herein by reference in its entirety, were maintained in MEM medium and 1.5 mg/ml of puromycin. MCF-7 human breast adenocarcinoma cells and 2008 human ovarian cancer cells were maintained in RMPI medium. U87MG human glioblastoma cells, SKBr3 human breast carcinoma cells, 1975 lung cancer cells, HUH-7 and 4-4 liver cancer cells and PC-3 human prostate cancer cells were cultured with DMEM.

### Example 3

### High-Throughput Screening (HTS) of Small Molecule Chemical Libraries Using 293T Cells Stably Expressing Hsp90(α/β)/p23 Split RL Reporters

To identify novel Hsp90 inhibitors, the library of pharmacologically active compounds (LOPAC1280®) was used. HTS was performed at Stanford High-throughput Bioscience Center (HTBC). 8×10³ 293T expressing Hsp90 α/p23 or Hsp90 β/p23 split RL reporters were plated in each well of the 384-white bottom plate from columns 1-22 (E & K Scientific, Santa Clara, Calif.) in 60 μl of medium using the Matrix Wellmate (Thermo Scientific, Hudson, N.H.) and allowed to attach for 24 h. 60 μl of medium (no cells) was added to columns 23-24 to control for background signals due to substrate alone. Approximately 100 nl of each LOPAC compound (10 mM stock in DMSO) was then added to each well in columns 3-22 (final concentration approximately 17 μM) using a PinTool (V&P Scientific, San Diego, Calif.) on a Sciclone ALH3000 (Caliper Sciences, Hopkinton, Mass.). Cells in columns 1-2 that were not treated with the compounds were used to determine the baseline signals from Hsp90(α/β)/p23 interactions.

Baseline complemented RL activities were determined 90 mins after addition of 20 μl of ENDUREN® (10 μM final concentration) (Promega, Madison, Wis.) using the Analyst GT (Molecular Device, Sunnyvale, Calif.), with an acquisition time of 0.2 sec per well. Cells were incubated with compounds for 24 h prior to re-determination of complemented RL activities, and normalized to the mean RL activities of untreated cells in columns 1-2. Another small molecule chemical library with 30,176 compounds (SPECS, Wakefield, R.I.) was also used in screening modulators of Hsp90(α/β)/p23 inhibitors (at 8.3 μM, one drug per well for initial screening). For dose response/confirmation assay the compounds were tested in duplicate in an 8 point (0.156-20 μM) dose response assay for RL activities for both Hps90(α/β)/p23 interactions. The effect of each compound on cell proliferation at 20 μM was determined using Cell Titer-Blue assay in duplicate wells.

### Example 4

### Determination of the Efficacy of Lead Compounds in Disruption of Hsp90(α/β)/p23 Interactions

To determine the effect of lead compounds on disruption of Hsp90(α/β)/p23 interactions in intact cells, 3.5×10⁴ 293T cells stably transfected with split RL reporters as described in Chan et al., (2008) Cancer Res. 68: 216-226, were plated in each well in the 96-well black wall plate (Costar, Corning, N.Y.) and allowed to attach for 24 h, prior to treatment with different concentrations of the lead compounds for 24 h. The 10 μg/ml of ENDUREN® (in 50 ml of cell culture medium) was added to each well for 1.5 h and RL activities were determined. BLI signals were normalized to that of cell number for each well (determined by Alamar Blue assay (Invitrogen, Carlsbad, Calif.)), prior to normalization to carrier control-treated cells.

### Example 5

### Optical CCD Imaging in Living Mice

Mice were gas anesthetized using isofluorane (2% in 100% oxygen, 1 L/min) during all injection and imaging procedures and kept at 37° C. Mice were imaged using a cooled CCD camera (IVIS 200, Caliper Life Sciences, Mountain View, Calif.). Tumor establishment and BLI of 293T cells stably expressing Hsp90(α/β)/p23 split RL reporters and FL-eGFP in 7-week-old female nude mice (nu/nu, Charles River) were performed as described as described in Chan et al., (2008) Cancer Res. 68: 216-226, incorporated herein b reference in its entirety. Baseline RL activities in the implanted tumors in living mice were determined by i.v. injection of 30 μg cltz (in 150 μl of 5% ethanol: 95% PBS) and image acquisition of 3 mins. After a 30 mins wait for RL signals to decay, FL activities were determined by i.v. injection of 163 μg of D-Luc in 100 μl PBS with image acquisition of 10 sequences for 15 sec each to obtain the peak max radiance. One set of mice were intraperitoneally (i.p.) injected with 80 mg/kg of CP9 dissolved in 100% DMSO in a final volume of 60 μl (n=5 per group). Another set of mice were treated with equal volume of DMSO as carrier control (n=5). At different time points post-treatment, follow-up RL and FL imaging was performed to monitor the effects of the CP9 on complemented Hsp90(α/β)/p23 interactions and cell proliferation. Maximum radiance of RL was divided by that of FL signals at each time point, prior to normalization to that of time 0 hr for each individual mouse, and expressed as average radiance±S.E.M for each treatment group.

Mice were euthanized after the last imaging time points and tumors were excised and homogenized in tissue extraction buffer in the presence of HALT® protease and phosphatase inhibitors (all from Pierce, Ill.). Protein concentrations were determined by Biorad Protein Dc assay. The expression of phosphorylated/total Akt, Raf-1 and β-tubulin were determined by western blotting as described in Chan et al., (2008) Cancer Res. 68: 216-226. Western blot images were quantitated using NIH Image J, and expressed as the ratio of target protein/α-tubulin for each treatment group.

### Example 6

### PET/CT Imaging of Glucose Metabolism in Living Mice

To determine the effects of CP9 on glucose metabolism in 293T xenografts stably expressing Hsp90(α/β)/p23 split RL reporters and FL-eGFP, baseline ¹⁸F-FDG uptake in each tumor site for each mouse were determined by small animal PET imaging using the Inveon PET/CT scanner (Siemens, Germany). Mice were placed on bed position first for CT image acquisition (632 slices at 206 um) that was used both for photon attenuation correction and image co-registration with PET image data for anatomical information. Static 5 min PET scan was then performed for [¹⁸F]-FDG activity and reconstructed using the Ordered Subsets Expectation Maximization (OSEM) 2D algorithm (159 slices with 1.5 mm resolution). ROI analysis was performed using the Inveon Research Workspace software. The maximum % injected doses per gram (% ID/g) upon normalization to injected dose were determined pre- and 43 h post-CP9 treatment.

### Example 7

### Data Analysis

Each experiment was repeated at least three times and results were expressed as mean+/−standard error of means (S.E.M.). Statistical differences were determined by student t-test using p<0.05 as cut-off point. HTS data was analyzed using MDL Assay Explorer.

### Example 8

### Elimination of Non-Specific Compounds

To determine if the lead hit compounds (19 total) were non-specific inhibitors of RL activities, 3×10⁴ 293T cells stably expressing full-length RL were plated in each 96-well black wall plate for 24 h. They were treated with different compounds at a 10-fold higher concentration than their respective IC₅₀ for inhibition of Hsp90α/p23 BLI signals for 4-24 h. Cells treated with carrier controls (0.1% DMSO) were used as the positive control. RL signals were determined using a cooled CCD camera upon addition of ENDUREN® (90 mins, final concentration of 10 μM) and normalized to that of carrier control treated cells. Compounds that led to more than 20% inhibition of RL signals were excluded from further analyses. Other toxic and non-specific compounds were eliminated based on database analyses of previous screening assays performed at HTBC.

To confirm the lead compound CP9 did not affect FL activities, 293T cells expressing full-length FL were treated with different concentrations of CP9 (1.6 to 25 μM) or carrier control for 4-38 h. PU-H71 (5 μM) and 17-AAG (50 μM) were used as controls. FL activity at different time points post CP9 treatment were determined by BLI upon addition of 1 ml of D-Luciferin in PBS (0.225 mg/ml).

### Example 9

### Western Blotting and Co-Immunoprecipitation

To validate the mechanisms of the lead compounds and their analogues for inhibition of Hsp90(α/β)/p23 interactions, 293T cells stably expressing the Hsp90(α/β)/p23 split reporters were treated with 5 μM of CP1, CP9 and CP18 or carrier control for 24 h. Cells treated with 2 μM of PU-H71 served as a positive control. Western blotting of Hsp70, phosphorylated and total Akt and co-immunoprecipitation of Hsp90(α/β)/p23 interactions was performed as described Inglese et al., (2006) Proc. Nat. Acad. Sci. U.S.A. 103: 11473-11478, incorporated herein by reference in its entirety. The expression of Raf-1 was determined using rabbit polyclonal antibodies against Raf-1 (0.5 μg/ml, Abcam).

### Example 10

### ³H-(Fluoro-2-deoxyglucose) [³H-FDG] and ³H-(3-Fluorodeoxy-thymidine) [³H-FLT] Cell Uptake Studies

To determine the downstream effects of lead compounds on glucose metabolism, 1.5×10⁵ 293T, 1975 and 2008 and MEFs cells plated in 24-well plate for 24 h, prior to treatment with different concentrations of CP9 for 24 or 48 h and subsequent incubation with 1 μCi of ³H-FDG in 500 μl of medium for 1 h at 37° C. Cells treated with 2.5 μM of PU-H71 served as a positive control. The total counts in each sample were normalized to that of the dose added to each well and to the amount of protein, and expressed as mean counts/dose/μg protein±S.E.M. Likewise, the effect of CP9 on cell proliferation was determined using 1 μCi of ³H-FLT per well after 2 h of incubation at 37° C.

### Example 11

### Purified Hsp90(α/β) Binding Assay

To verify the binding of CP9 to Hsp90α and Hsp90β in vitro, a displacement assay using ³H-17AAG was performed. 1 mg of purified Hsp90α or Hsp90β was incubated with different concentrations of CP9 or DMSO carrier control in 50 μl of HBS-P+ binding buffer (Biacore, Piscataway, N.J.) for one h on ice on a shaking platform. 17-AAG (final concentration of 200 μM) was used as a positive control. Duplicate samples were used for each condition. 1 μl of ³H-17AAG (final concentration of 0.5 μM) was added and the mixture was incubated for another 30 min at room temperature. Unbound ³H-17AAG and inhibitors were removed using the 7 kDa Zeba desalt column (Thermo Scientific, Rockford, Ill.). The amount of ³H-17AAG that remained bound to Hsp90α or Hsp9013 was determined by scintillation counting.

### Example 12

### Displacement of ³H-17AAG Uptake by CP9 in HT29 Cells

To determine if CP9 binds to cellular Hsp90, an uptake study of ³H-17AAG (Moravek Biochemicals, Brea, Calif.) was performed. 1×10⁵ HT29 cells in 500 μl of medium were plated in each 24-well plate and allowed to attach for 24 h. Cells were incubated with 0.5 μM ³H-17AAG in the presence of 0.4 to 12.5 μM CP9 or carrier control for 1 h at 37° C. 5 μM of PU-H71 was used as a positive control. This was followed by 2 washes with 500 μl of PBS and one h of wash-out in 500 μl medium to remove unbound ³H-17AAG. Cells were lysed in 300 μl of T-per tissue extraction buffer, in the presence of protease and phosphatase inhibitors (Thermo Scientific, Rockford, Ill.) on ice for 15 mins and cell lysates were prepared for scintillation counting and protein determination. The total counts in each sample were normalized to that of the dose added to each well and to the amount of protein, and expressed as mean counts/dose/μg protein±S.E.M.

### Example 13

### Generation of 293T Stable Cells Expressing Hsp90(α/β/p23 Split RL Reporters and eGFP-FL Fusion Reporter [Hsp90(α/β/p23-FG]

To monitor the effect of different Hsp90 inhibitors on cell proliferation, a second imaging reporter expressing a Firefly Luciferase-enhanced green florescent protein fusion protein (eGFP-FL) under the control of an ubiquitin-C promoter was used. The effect of different inhibitors on Hsp90(α/β)/p23 interactions was monitored by RL imaging, whereas their effects on cell proliferation were monitored by FL imaging since their respective substrates (coelentarazine and D-Luciferin) do not cross-react. This eGFP-FL reporter was introduced into 293T cells stably expressing Hsp90α/p23 or Hsp90β/p23 split RL reporters via lenti-viral transduction. Single cell colonies were selected by plating cells at low densities (1000-3000 cells per 10 cm² dish) and FL imaging (1 min using a cooled CCD camera) upon addition of D-Luciferin in PBS (0.225 mg/ml final concentration).

### Example 14

### Screening of Structural Analogues of Lead Compound CP9

To identify more potent structural analogues of CP9, 293T cells stably express Hsp90(α/β)/p23 split RL reporters and FL-eGFP fusion reporters were treated with 62 different CP9 analogues (Asinex, Russia) at 10 μM for 24 h in duplicate wells. CP9 (0.3-10 μM), 17-AAG (10 μM) and PU-H71 (5 μM) were used as positive controls in triplicate wells. The efficacy of the analogues in disruption of Hsp90(α/β)/p23 and cell proliferation were monitored by RL imaging (2 h post upon addition of ENDUREN® at 30 μM final concentration) followed by FL imaging (10 mins post addition of D-Luc at 0.225 mg/ml final concentration) using a cooled CCD camera (1 min and 10 sec for RL and FL imaging, respectively). RL signals were normalized to that of FL signals to account for the effect of the analogues on cell proliferation. Dose response curves (six 2-fold serial dilutions) for disruption of Hsp90(α/β)/p23 in the stable cells were also generated for the top 8 compounds (A17, A15, A29, A31, A39, A58, A61, A65) and compared to that of CP9. The effect of the CP9 analogues on degradation of Hsp90 client proteins in 293T cells were also monitored by western blotting as described above.

### Example 15

TABLE 1
Effect of the lead compounds (CP1-CP19) on inhibition of
Hsp90α/p23, Hsp90β/p23 interactions and cell proliferation.

| | IC₅₀ | IC₅₀ | % Inhibition of Cell |
|---|---|---|---|
| | (Hsp90α/p23) - | (Hsp90β/p23) - | Proliferation |
| Compounds | μM | μM | (20 μM) |
| CP1 | 0.2 | 0.5 | 44 |
| CP2 | 0.8 | 2.4 | 45 |
| CP3 | 1.2 | 1.6 | 33 |
| CP4 | 1.4 | 1.7 | 46 |
| CP5 | 1.4 | 3.1 | 35 |
| CP6 | 1.8 | 2.8 | 43 |
| CP7 | 1.9 | 2.9 | 45 |
| CP8 | 2.7 | 6.0 | 32 |
| CP9 | 3.2 | 15.3 | 29 |
| CP10 | 1.2 | 2.4 | 11 |
| CP11 | 1.4 | 2.3 | 10 |
| CP12 | 1.4 | 2.4 | 21 |
| CP13 | 2.1 | 3.3 | 27 |
| CP14 | 2.1 | 8.1 | 10 |
| CP15 | 2.3 | 5.7 | 27 |
| CP16 | 2.5 | 4.0 | 2 |
| CP17 | 3.9 | 17.5 | 13 |
| CP18 | 0.07 | 0.04 | 55 |
| CP19 | 0.3 | 0.8 | 50 |

IC₅₀ was defined as the concentration of the compound required to inhibit bioluminescence signals by 50%, relative to carrier control treated 293T cells stably expressing Hsp90α/p23 split RL reporters. IC₅₀ was defined as the concentration of the compound required to inhibit bioluminescence signals by 50%, relative to carrier control treated 293T cells stably expressing Hsp90β/p23 split RL reporters. % Inhibition of 293T cells stably expressing Hsp90</p23 split RL reporters by each compound at 20 μM was determined by CELL TITER BLUE ASSAY®.

### Example 16

FIGS. 1A-1D illustrate the chemical structures of the compounds (CP1-19) isolated by HTS screening using the method of the disclosure.

### Example 17

FIGS. 2A-2G illustrate the structural analogues of the lead compound CP9. The compounds A1-A62 (2-(trifluoromethyl)pyrimidin-2-yl)thio)acetamide derivatives) were rationally selected for structural activity relationship (SAR) studies.

We claim:

1. A therapeutic composition comprising an inhibitor of an Hsp90 chaperone activity, wherein the inhibitor is selected from the group consisting of: compounds CP1-CP19 as shown in FIGS. 1A-1D and a compound having the formula I: 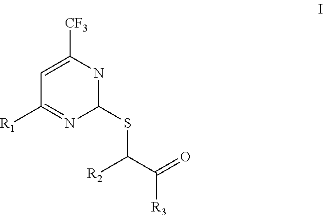
wherein R₁ is a thiophene, a furan, a substituted or unsubstituted phenyl, or —OH;
R₂ is H or an alkyl; and
R₃ is phenylmethylamine, 4-amidopyridyl, or —NHR₄, wherein R₄ is a substituted isoxazole, a substituted or unsubstituted alkyl, a substituted or unsubstituted branched chain alkyl, a substituted or unsubstituted —(CH)ₙPh, a substituted or unsubstituted 5 or 6-membered aryl, a substituted or unsubstituted 5 or 6-membered heteroaryl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine,
and wherein the therapeutic composition is formulated to have a dose of the inhibitor effective in reducing the viability of a cancer cell when delivered to an animal or human.

2. The therapeutic composition of claim 1, wherein R₁ is a thiophene, a furan, or a substituted phenyl, wherein the substituted phenyl is a methoxyphenyl, an halogenated phenyl, or a dimethoxyphenyl.

3. The therapeutic composition of claim 1, wherein the inhibitor has the formula I and is selected from compounds CP9 and A1-A62 of FIGS. 2A-2G.

4. The therapeutic composition of claim 1, wherein the inhibitor has the formula I: 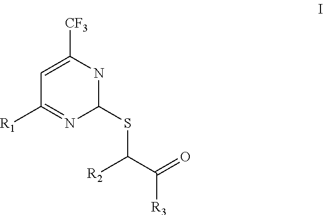
wherein R₁ is a thiophene, a furan, phenyl, a substituted phenyl, or —OH;
R₂ is H or methyl; and
R₃ is phenylmethylamine, 4-amidopyridyl, or —NHR₄, wherein R₄ is a substituted isoxazole, an alkyl, a branched chain alkyl, a —(CH)ₙPh, a substituted —(CH)ₙPh, -Ph, a substituted phenyl, a substituted biphenyl, a cycloalkyl, a pyridyl, —(CH)ₙpyridyl, methylfuranyl, a methyltetrahydrofuran, a substituted pipenazyl, or a morpholine, and wherein n=1 or 2,
and wherein the therapeutic composition is formulated to have a dose of the inhibitor effective in reducing the viability of a cancer cell when delivered to an animal or human.

5. The therapeutic composition of claim 4, wherein R₁ is a thiophene, a furan, phenyl, or a substituted phenyl, wherein the substituted phenyl is a methoxyphenyl, an halogenated phenyl, or a dimethoxyphenyl.

6. The therapeutic composition of claim 4, wherein the inhibitor is selected from compounds CP9 and A1-A62 of FIGS. 2A-2G.

7. The therapeutic composition of claim 4, wherein the inhibitor is N-(5-methylisoxazol-3-yl)-2-(4-(thiophen-2-yl)-6-(trifluoromethyl)pyrimidin-2-ylthio)acetamide (CP9) having the formula:

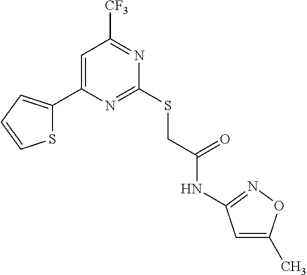 [SMILES: Cc1cc(NC(=O)CSc2nc(-c3cccs3)cc(C(F)(F)F)n2)no1]

8. The therapeutic composition of claim 1, wherein the inhibitor is CP9, A17, A29, or A61, or a combination thereof.

9. The therapeutic composition of claim 1 further comprising a pharmaceutically acceptable carrier.

10. A method of reducing the viability of a cancer cell in an animal or human, the method comprising delivering to the animal or human a therapeutically effective amount of an inhibitor of an Hsp90 chaperone activity, wherein the inhibitor is selected from the group consisting of: compounds CP1-CP19 as shown in FIGS. 1A-1D and a compound having the formula I: 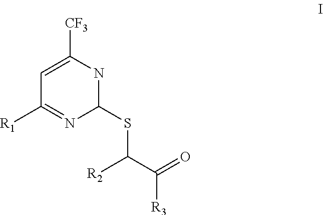
wherein R₁ is a thiophene, a furan, a substituted or unsubstituted phenyl, or —OH;
R₂ is H or an alkyl; and
R₃ is phenylmethylamine, 4-amidopyridyl, or —NHR₄, wherein R₄ is a substituted isoxazole, a substituted or unsubstituted alkyl, a substituted or unsubstituted branched chain alkyl, a substituted or unsubstituted —(CH)ₙPh, a substituted or unsubstituted 5 or 6-membered aryl, a substituted or unsubstituted 5 or 6-membered heteroaryl, a substituted or unsubstituted biphenyl, a substituted or unsubstituted cycloalkyl, a substituted or unsubstituted pyridyl, a substituted or unsubstituted —(CH)ₙpyridyl, a substituted or unsubstituted methylfuranyl, a substituted or unsubstituted methyltetrahydrofuran; a substituted or unsubstituted pipenazyl, or a morpholine,

11. The method of claim 10, wherein R₁ is a thiophene, a furan, phenyl, or a substituted phenyl, wherein the substituted phenyl is a methoxyphenyl, an halogenated phenyl, or a dimethoxyphenyl.

12. The method of claim 10, wherein the inhibitor has the formula I and is selected from compounds CP9 and A1-A62 of FIGS. 2A-2G.

13. The method of claim 10, wherein the inhibitor has the formula I: 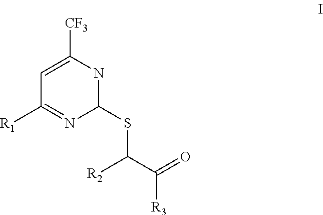
wherein R₁ is a thiophene, a furan, phenyl, a substituted phenyl, or —OH;
R₂ is H or methyl; and
R₃ is phenylmethylamine, 4-amidopyridyl, or —NHR₄, wherein R₄ is a substituted isoxazole, an alkyl, a branched chain alkyl, a —(CH)ₙPh, a substituted —(CH)ₙPh, -Ph, a substituted phenyl, a substituted biphenyl, a cycloalkyl, a pyridyl, —(CH)ₙpyridyl, methylfuranyl, a methyltetrahydrofuran; substituted pipenazyl, or a morpholine, and wherein n=1 or 2,
and wherein the therapeutic composition is formulated to have a dose of the inhibitor effective in reducing the viability of a cancer cell when delivered to an animal or human.

14. The method of claim 13, wherein R₁ is a thiophene, a furan, phenyl, or a substituted phenyl, wherein the substituted phenyl is a methoxyphenyl, an halogenated phenyl, or a dimethoxyphenyl.

15. The method of claim 13, wherein the inhibitor is selected from compounds CP9 and A1-A62 of FIGS. 2A-2G.

16. The method of claim 13, wherein the inhibitor is N-(5-methylisoxazol-3-yl)-2-(4-(thiophen-2-yl)-6-(trifluoromethyl)pyrimidin-2-ylthio)acetamide (CP9) having the formula:

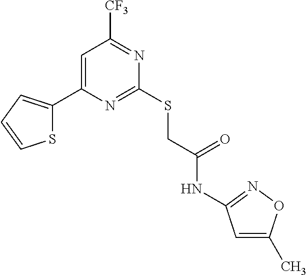 [SMILES: Cc1cc(NC(=O)CSc2nc(-c3cccs3)cc(C(F)(F)F)n2)no1]

17. The method of claim 10, wherein the inhibitor is CP9, A17, A29, or A61, or a combination thereof.

18. A high-throughput method for identifying an inhibitor of Heat Shock Protein 90 (Hsp90) chaperone activity, the system comprising:
(a) obtaining a genetically modified cell, or progeny thereof expressing a split luciferase reporter configured to provide a detectable signal on binding of a p23 polypeptide and a Heat Shock Protein 90 (Hsp90) polypeptide in the presence of coelentarazine;
(b) detecting a first detectable signal emitted from the genetically-modified cell or population thereof;
(c) contacting the genetically-modified cell or progeny thereof with a compound suspected of being an Hsp90 inhibitor;
(d) detecting a second detectable signal emitted from the genetically-modified cell or progeny thereof expressing the split Renilla luciferase reporter; and
(e) comparing the intensities of the first and the second detectable signals, whereby if the intensity of the first detectable signal is greater than intensity of the second detectable signal, the compound is determined to inhibit the formation of a complex between p23 and an Hsp90 polypeptide.

19. The method of claim 18, wherein the method further comprises the steps:
(f) obtaining a subject animal comprising a xenograft tumor derived from the genetically-modified cell of step (a);
(g) administering to the animal coelentarazine and detecting a third detectable signal intensity from the xenograft tumor; and
(h) administering to the subject animal the compound determined in step (e) to inhibit complex formation between p23 and an Hsp90 polypeptide, and coelentarazine, and obtaining a fourth detectable signal intensity from the xenograft, wherein if the fourth signal intensity is less than the third signal intensity, the compound identified in step (e) is identified as an inhibitor of complex formation between p23 and an Hsp90 polypeptide in vivo.

20. The method of claim 18, wherein the split luciferase reporter comprises a p23 polypeptide having N-terminus fragment of a Renilla luciferase attached thereto, and an Hsp90 polypeptide having a C-terminus fragment of the Renilla luciferase attached thereto, whereby when the p23 and the Hsp90 polypeptides are in contact in the presence of ATP the N- and C-termini of the Renilla luciferase cooperate to generate the first detectable signal in the presence of coelentarazine.

21. A kit comprising a container containing a therapeutic composition comprising a compound of FIGS. 1A-1D and 2A-2G, or a pharmaceutically effective derivative thereof and instructions for administering the compounds or formulations to a subject.
